(19) United States
(12) Patent Application Publication
(10) Pub. No.: US 2010/0310635 A1
(43) Pub. Date: Dec. 9, 2010

(54) Compositions and Methods for Regulating T-Cell Activity

(76) Inventors: Engin Ozkaynak, Wynnewood, PA (US); Wayne W. Hancock, Philadelphia, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/597,687

(22) PCT Filed: Apr. 25, 2008
(86) PCT No.: PCT/US08/61595

§ 371 Date: Aug. 23, 2010

Related U.S. Application Data
(60) Provisional application No. 60/913,960, filed on Apr. 25, 2007.

(51) Int. Cl. A61K 9/127; C12Q 1/37; A61K 31/7088; G01N 33/566; C07K 16/00; C07H 21/00; C07K 7/08; C07K 14/435; A61K 39/00; A61K 39/395; A61K 38/10; A61K 38/17; A61K 35/12; A61P 37/02

(52) U.S. Cl. 424/450; 435/23; 514/44 A; 435/7.1; 530/387.9; 536/23.1; 530/327; 530/350; 530/324; 514/44 R; 424/184.1; 424/172.1; 514/21.5; 514/21.3; 514/21.2; 424/93.7; 436/501

(57) ABSTRACT

Methods, compositions and kits effective for modulating and immunomonitoring of Treg activity are provided. Therapeutic methods involving formation and uses of cleaved Foxp3 are disclosed, as well as screening assays for identifying agents effective for modulating Treg activity.

This application claims priority under 35 U.S.C. §119(e) to U.S. Provisional Patent Application 60/913,960, filed on Apr. 25, 2007. The foregoing application is incorporated by reference herein.

### FIELD OF THE INVENTION

The present invention relates to the fields of molecular biology and immunology. More specifically, the invention provides compositions and methods useful for modulating and monitoring regulatory T cell (Treg) activity, particularly in patients with autoimmune disease or cancer.

### BACKGROUND OF THE INVENTION

Several publications and patent documents are cited throughout the specification in order to describe the state of the art to which this invention pertains. Full citations for these publications are found at the end of the specification. Each of these citations is incorporated by reference herein as though set forth in full.

Foxp3 is a 47 kDa DNA-binding protein specific to regulatory T cells (Tregs). Mutations in Foxp3 result in IPEX syndrome. IPEX syndrome (immune dysregulation, polyendocrinopathy, enteropathy, X-linked), also known as XLAAD (X-linked autoimmunity and allergic dysregulation) syndrome, was first recognized in 1982 by Powell et al (5, 6). IPEX is a potentially lethal autoimmune disorder due to a mutation in the forkhead box P3 (Foxp3) gene located on the X-chromosome (7). Heterozygous females are healthy since the normal and mutated Foxp3 alleles are expressed equally. However, most male patients are severely affected and die early in life. Common features of IPEX are severe and chronic diarrhea, type I diabetes, failure to thrive, thyroiditis, eczema, alopecia, anemia, autoimmune hepatitis, hepatomegaly and lymphadenopathy (8). Histology shows infiltration of lymph nodes, spleen, liver, pancreas and skin by activated T cells. A natural spontaneous mutation, first described in scurfy mice in 1949, results in a phenotype which is similar to the human IPEX syndrome. The scurfy mutation was located by positional cloning to scurfin (sf), the mouse ortholog of the human Foxp3 gene (9). In male mice, signs appear shortly after birth, and like in humans, include skin rashes, diarrhea and malabsorption, with hepatosplenomegaly, lymphadenopathy, massive lymphocytic infiltrates in many organs, and death by about 3 weeks of age. In scurfy mice, the murine Foxp3 gene which normally encodes a 429 aa (48-kDa) protein was found to have a 2-bp insertion, resulting in a premature stop codon, and hence a shorter protein (9).

The Fox family has 17 subfamilies (FoxA-FoxQ), and Fox family proteins play important roles in biological processes ranging from development to speech to autoimmunity and cancer. Foxp3 is critical to the regulation of immune responses. Unlike other Fox protein family members which activate genes, some FoxP subfamily members repress transcription of genes and this property has also been shown for Foxp3, the shortest member of the FoxP sub-family (22, 23). Foxp3 lacks the glutamine rich regions present in Foxp1, -2, and -4 and has a much shorter carboxyl-terminal extension beyond the FKH domain (only 12-amino acids). The glutamine rich regions of Foxp1, -2, and -4 have been shown to be directly involved in transcriptional repression (24), however, the absence of any glutamine-rich region suggests Foxp3 represses transcription by another mechanism.

Foxp3 also has domains that are present in other members of the Foxp family, such as the zinc-finger and leucine zipper domains in the mid-portion of the molecules. Foxp1, -2, and -4 form homo- and heterodimers (25) and deletion of a single glutamic acid (residue 251; location exon7) in the leucine zipper prevents oligomerization, DNA-binding and transcriptional repression (26). By analogy with the other proteins (27), the zinc-finger domain of Foxp3 may also function in dimerization. Analysis of Foxp3-deficient and Foxp3-transgenic mice showed the important role this protein plays in the development of CD4+CD25+ Tregs. Foxp3-deficient mice lack Tregs and have increased autoreactive T cells, similar to that of Scurfy mice, while Foxp3-transgenic mice have an increased CD4+CD25+ population (21, 28). Ectopic expression of Foxp3 by non-Tregs confers suppressor function, in conjunction with repression of endogenous IL-2 and IFN-γ production and induction of CD25, GITR and CTLA-4 expression (18, 29, 30).

Work by Bettelli showed the direct interaction of Foxp3 with NF-AT and the p65 subunit of NF-κB (31). NF-κB-mediated transcriptional activation was inhibited by Foxp3, while the NF-δB DNA-binding was unaffected. Formation of a complex between NFAT and Foxp3 was recently shown to be important for Foxp3 function, with graded mutations in NFAT-interacting residues in the FKH domain resulting in progressive loss of function (29). This study also focused on the role of the N-terminal Foxp3 sequences. An N-terminal deletion mutant (remaining residues: 182-431) retained its NFAT and DNA binding abilities while losing its abilities to suppress IL-2 and upregulate CD25 and CTLA-4 expression, indicating N-terminal sequences can be involved in recruiting transcriptional corepressors and coactivators. Functional association of Foxp3 with histone deacetylases is supported by the demonstration that Foxp3 binding to the IL-2 and IFN-γ promoters result in marked histone deacetylation and conversely binding to the GITR, CD25, and CTLA-4 promoters leads to increased histone acetylation (30).

Exon2/exon7 deleted Foxp3 isoforms missing some N-terminal sequences and most of the leucine zipper sequences, or both, were found to be functional at about the same level as the wt-Foxp3 in suppressing CD28/TCR chimeric receptor-induced IL-2 production in transfected human CD4+ T cells (3) indicating either certain Foxp3 functions can be carried out in monomer form or that certain precise deletions, as in the isoforms, may lead to structural changes leading to dimerization despite critical domain loss. Foxp3 may function in certain instances as a monomer, and formation of multimeric complexes seems to be a unique property of FoxP sub-family since other Fox proteins can bind DNA as monomers (26). The zinc-finger domain of Foxp3 may assume such a role in Foxp3 isoforms since zinc-fingers have been reported to enable dimerization for several proteins (32).

Foxp3 gene expression is detected at high levels in spleen, thymus and lymph nodes. Foxp3 is mainly expressed by CD4+CD25+ Tregs but CD4+CD25− T cells also show limited Foxp3 expression (9, 18). Both CD4+CD25+ natural Tregs and “induced” Treg cells (induced by allo- or antigen-priming) express high levels of Foxp3 (19, 20). Retroviral expression of Foxp3 in CD4+CD25− cells results in their conversion to a phenotype very similar to Tregs with the ability to suppress effector T cell functions (18). Retroviral expression of Foxp3 protects host mice from CD4+CD25− cell-introduced autoimmune gastritis (21). In humans, 2 isoforms of Foxp3 (Foxp3 and Foxp3α) are described. Foxp3α is generated as a result of alternative splicing at exon 2 and encodes a shorter protein of 396 aa, lacking 35 aa from the region corresponding to aa 71-105 (20). Different sources of T cells were found to express both isoforms, but data from individual T cell clones show the two Foxp3 forms are differentially expressed with the individual clones expressing either one of the two isoforms (20). More recently, in addition to the exon2 deleted Foxp3 isoform, a exon2/exon7 deleted isoform has also been reported (3).

Targeted deletion of Foxp3 results in a scurfy-like syndrome, providing a second line of evidence to show the culprit in scurfy mice (and in IPEX patients) to be the non-functional Foxp3. The autoimmune syndrome observed in the scurfy mice is due to the absence of Treg cells; CD4+ T cells from these animals are hyper-responsive to stimuli and produce a variety of cytokines (9, 10). The scurfy phenotype resembles that of mice deficient in CTLA-4 or TGF-β, and adoptive transfer of CD4+ cells from scurfy mice into SCID or nu/nu results in the rapid onset of a wasting disease (11). The frame-shift mutation in Scurfy mice results in the loss of the C-terminal DNA-binding forkhead (FKH) domain and lethal autoimmunity by 2-3 weeks of birth (9), whereas in humans, multiple mutations can lead to disease (12). Similar to the results obtained earlier with purified CD4+CD25+ Tregs (13), transfer of TCR transgenic BDC2.5 cells retrovirally expressing Foxp3 into nonobese diabetic (NOD) mice prevents progression of diabetes (14).

Following the murine results, IPEX patients were screened for mutations in the Foxp3 gene and various mutations were found in the >20 families studied, confirming that the loss of Foxp3 function results in the IPEX syndrome. The human Foxp3 gene is located at Xp11.23 and has 11 coding exons. The structural domains of Foxp3 include a Zinc-finger, a Leucine-zipper and a forkhead domain. Sequence analysis of the Foxp3 gene in IPEX patients revealed a large portion of the patients having missense mutations (15). Most of the reported mutations are clustered in the Leucine zipper region and the Forkhead domain, highlighting the importance of these regions in the Foxp3 function (8). However, mutations corresponding to other parts of the Foxp3 gene such as a mutation in an intron (8), the polyadenylation signal sequence (AATAAA→AATGAA), and mutations at the N-terminal side of the protein (16, 17) are reported. At least two mutations at the C-terminal domain of Foxp3 (past the Forkhead domain) highlight the importance of the C-terminal sequences in the function of Foxp3 since both result in severe IPEX syndrome (7, 12). One of these mutations is not in the coding region but abolishes the stop codon and adds 25 amino acids to Foxp3. The second C-terminal mutation results in the loss of the last Foxp3 amino acid while extending the protein by 21 amino acids.

Overall, the immune system must discriminate between non-self and self in order to function properly. When this discrimination fails, the immune system destroys tissues and cells of the body (i.e., autoimmune disease). On one hand, it is the job of Tregs to suppress the activation of the immune system to prevent pathological self-reactivity. The potential for Tregs to actively regulate autoimmunity and induce long term tolerance has therapeutic potential as a strategy for inducing prolonged tolerance. In light of the critical role Tregs play in regulating the immune response, and the current lack of a reliable means to regulate and detect Treg activity, a need exists for compositions and methods to modulate and monitor Treg activity in order to ascertain whether the immune system is functioning properly. On the other hand, methods are needed to boost the immune response to cancer by specifically directing the response to target cancerous cells while avoiding deleterious effects on normal cells. It has been shown for several cancers that immunological factors affect patient outcomes, indicating the state of the immune system plays an important role in recurrence and mortality.

In ovarian cancer, Treg infiltration (and Foxp3 expression) into tumors is correlated with poor clinical prognosis. Using a cohort of 70 ovarian cancer patients, Curiel et al. has shown that levels of intratumoral Tregs inversely correlate with survival (77) and high levels of Foxp3 expression result in poor prognosis (78). Increased tumor grade in human gliomas correlate with CD4+CD25+Foxp3+ T cell levels (79) and in mice with experimental brain tumors, depletion of the CD4+CD25+ Treg population results in prolongation of survival (80). In non-small lung cancer (NSCLC), where patient relapse is high (about 50% even if diagnosis is made early) the ratio of Tregs to tumor infiltrating T cells (TIL) has recently been shown to be important in predicting recurrence. Stage I NSCLC patients with a high proportion of tumor Treg cells relative to TIL have a significantly higher risk of recurrence (81). Analysis of Foxp3 expression in pancreatic carcinoma cells showed Foxp3 is expressed by the malignant epithelial cells, in addition to the infiltrating Treg cells. Pancreatic cancer cell lines showed Foxp3 expression is inducible by TGF-β2 (82). Foxp3 expression by the pancreatic ductal adenocarcinoma cells and tumors indicate local mechanisms, in addition to the Tregs, also contribute to the immune evasion and tumor progression (82).

Since high Treg numbers are associated with the ability of tumor cells to evade the host immune response, depletion or inhibition of Tregs leads to a more robust anti-tumor immune response. (reviewed in: Zou et al. Nat, Rev. Immunol. (2006) 6:295-307). It is critical to determine how to overcome the suppressive activity of Tregs to induce effective tumor-specific immune responses capable of controlling and destroying tumors. The present invention satisfies this need and provides related advantages as well.

### SUMMARY OF THE INVENTION

The present inventor has discovered that cleavage products of Foxp3 are effective modulators of Treg function and activity. Thus, in accordance with the present invention, a method for identifying agents which affect the formation of cleaved Foxp3 are provided. An exemplary method comprises administering an agent to a cell expressing Foxp3 and enzymes responsible for cleavage thereof, determining levels of Foxp3 cleavage product, if any, relative to an untreated cell, and identifying those agents which modulate the formation of said Foxp3 cleavage product. Such agents should have therapeutic value. Agents so identified can be effective to alter the expression level or function of a proprotein convertase enzyme, thereby modulating Foxp3 activity and/or Treg function. In a one embodiment, the agent is effective to increase formation of a peptide selected from the group consisting of SEQ ID NO: 8, SEQ ID NO: 11, SEQ ID NO: 15, SEQ ID NO: 20 and SEQ ID NO: 24. Alternatively, the agent may be effective to decrease formation of the above-mentioned peptides.

In another embodiment of the invention, a method of treating autoimmunity is provided. An exemplary method entails administering an effective amount of at least one peptide selected from the group consisting of SEQ ID NO: 11, SEQ ID NO: 15, and SEQ ID NO: 24, to a patient in need thereof, said peptides optionally being contained in a liposome. The method optionally includes ex vivo administration of the peptide to isolated cells for a time sufficient to stimulate Treg production, after which said cells are reinfused into the patient.

The invention also includes isolated nucleic acids encoding the Foxp3 peptides of the invention as well as the isolated peptides encoded thereby. Antibodies immunologically specific for Foxp3 or functional fragments thereof, also comprise an aspect of the invention.

In yet another embodiment, a method for assessing regulatory T cell activation in a test subject is disclosed. An exemplary method entails providing a biological sample obtained from said test subject, the sample comprising Foxp3 protein; contacting the sample with an agent having binding affinity for said Foxp3 protein or a fragment thereof, comparing the amount of said Foxp3 protein or fragment thereof from said test subject with levels of Foxp3 protein or fragment thereof obtained from a normal subject, wherein an alteration of in the amount of Foxp3 protein or fragment thereof in the sample, relative to the normal subject is indicative of altered regulatory T cell activation. The method may optionally entail the steps of assessing inflammatory cytokine levels in the sample or identifying T cell specific markers present on T cells in said sample. In one embodiment of this method, regulatory T cell activation in a test subject is assessed by comparing the expression ratio of SEQ ID NO: 15 to SEQ ID NO: 2.

In yet another aspect, the invention provides a method for the treatment of an autoimmune disease in a patient in need thereof. In one approach, Treg activity is increased via introduction of an effective amount of at least one nucleic acid encoding a peptide selected from the group consisting of SEQ ID NO: 11, SEQ ID NO: 15 and SEQ ID NO: 24 in a target cell.

In a further embodiment the invention provides kits for practicing the methods described above.

Finally, siRNA molecules contained in a pharmaceutically acceptable carrier which are effective to down regulate the expression of a proprotein convertase enzyme involved in Foxp3 cleavage are provided. Such molecules should have therapeutic value for the treatment of cancer.

### BRIEF DESCRIPTION OF THE DRAWINGS

FIG. 1. Detection of Foxp3 C-terminal epitope tag by Western blot analysis. Foxp3 with a C-terminal Flag-epitope tag was retrovirally expressed. A total cell extract and an insoluble nuclear fraction were probed for the Flag-tag and Foxp3 protein levels.

FIG. 2. (A) Amino acid sequence of full-length mouse Foxp3 protein (SEQ ID NO: 1) and full-length human Foxp3 (SEQ ID NO: 2). The shaded areas designate the structural domains of the genes. Mouse Foxp3 has 86% identity to human Foxp3. (B) Primary sequence of full-length mouse Foxp3 cDNA (SEQ ID NO: 3). (C) Human full-length Foxp3 cDNA (SEQ ID NO: 4). (D) Mouse and Human Foxpeptide cDNA and amino acid sequences (SEQ ID NO: 5-8). (E) Mouse short-Foxp3 protein sequence (SEQ ID NO: 9) and mouse short-Foxp3 cDNA sequence (SEQ ID NO: 10). (F) Human short-Foxp3 protein sequence (SEQ ID NO: 11) and human short Foxp3 cDNA sequence (SEQ ID NO: 12).

FIG. 3. Detection of Foxpeptide by Western blot analysis. (A) Spleen and heart proteins were separated by SDS-20% PAGE and blotted onto PVDF membrane and analyzed by Western blotting using an antibody raised against a synthetic 11-mer peptide mimicking the carboxyl-terminal end of Foxp3 (see Experimental Procedures). (B) The size of the fragment detected by Western blot analysis of the spleen extract shows the expected size of a fragment that would be generated by proteolytic cleavage immediately after 414RKKR417 (SEQ ID NO: 179).

FIG. 4. Determination of Furin, PC1, PC7 and Foxp3 mRNA expression by qPCR in resting versus activated murine CD4+CD25− and CD4+CD25+ cells. Cells harvested from spleen and lymph nodes were purified over MACS columns, cultured with or without CD3 mAb for 3 d; values are relative to 18S ribosomal RNA, and p<0.01 for resting vs. activated levels of Furin, PC1 and PC7 (mean±SD).

FIG. 5. Detection of the cleaved and uncleaved forms of Foxp3 on Western blots. (A) Schematic of the constructs used: WT-Foxp3, short-Foxp3 and C-terminal-extended Foxp3. (B) Western blot analysis of C-terminal-extended Foxp3 in differing cellular fractions: lane 1, WT-Foxp3 (control); lane 2, short-Foxp3 (control); lanes 3-6, C-terminal-extended Foxp3, with lane 3, chromatin-bound fraction; lane 4, nuclear extract; lane 5, cytoplasmic extract; and lane 6, total cellular extract. Abbreviations are T, total extract; “chro-b”, chromatin-bound; N, nuclear extract; and C, cytoplasmic extract. Lanes 7-9, acetic acid urea electrophoresis (staining). DNA and protein content of chromatin fraction (chr), nuclear (N) and cytoplasmic (C) extracts are shown below lanes 3, 4, and 5. WT and short Foxp3 (RKKR•) (SEQ ID NO: 179) used as size controls.

FIG. 6. Sequences of N- and C-terminal cleaved (i.e., single or double cleaved) Foxp3. (A) Amino acid sequence of double cleaved mouse Foxp3 protein (SEQ ID NO: 13). (B) Primary sequence of double cleaved mouse Foxp3 cDNA (SEQ ID NO: 14). (C) Amino acid sequence of human double cleaved Foxp3 protein (SEQ ID NO: 15). (D) Primary sequence of human double cleaved Foxp3 cDNA (SEQ ID NO: 16). (E) Primary sequence of mouse Foxp3 N-terminal cleavage product (SEQ ID NO: 17). (F) Amino acid sequence of mouse Foxp3 N-terminal cleavage product (SEQ ID NO: 18). (G) Primary sequence of human Foxp3 N-terminal cleavage product (SEQ ID NO: 19). (H) Amino acid sequence of human Foxp3 N-terminal cleavage product (SEQ ID NO: 20). (I) Primary sequence of mouse Foxp3 singly cleaved at the N-terminus (SEQ ID NO: 21). (J) Amino acid sequence of mouse Foxp3 singly cleaved at the N-terminus (SEQ ID NO: 22). (K) Primary sequence of human Foxp3 singly cleaved at the N-terminus (SEQ ID NO: 23). (L) Amino acid sequence of human Foxp3 singly cleaved at the N-terminus (SEQ ID NO: 24).

FIG. 7. Different mFoxp3 forms resulting from cleavage at the N- and C-terminal RXXR (SEQ ID NO: 180) motifs. (A) Uncleaved Foxp3. (First row: SEQ ID NO: 180, Second row: SEQ ID NO: 191, SEQ ID NO: 188). (B) C-terminal cleaved, “short”-Foxp3. (SEQ ID NOs: 191 and 179). (C) N-terminal cleaved Foxp3, with C-terminus intact. (SEQ ID NOs: 183, 188). (D) Double cleaved Foxp3, N- and C-terminal cleavage. (SEQ ID NOs: 183, 179). (E) Expected sizes of fragments resulting from Foxp3 processing at the RXXR (SEQ ID NO: 180) sites. (SEQ ID NO: 183). Panels A-D are also applicable to human Foxp3, with the sole caveat that the C-terminal cleavage product (i.e., “Foxpeptide”) is 14-amino acids long in humans, while Foxpeptide is 12-amino acids long in the mouse.

FIG. 8. Dependence of Foxp3 proteolytic processing on an intact C-terminal 414RXXR417 (SEQ ID NO: 180) PC recognition motif. (A) Schematic of the three C-terminal extended constructs (RKKR-- (SEQ ID NO: 179), QNKR--- (SEQ ID NO: 181), QNKS--- (SEQ ID NO: 182)) used in retroviral expression. The arginine residues of the RXXR (SEQ ID NO: 180) motif are underlined and short and WT constructs were used as size controls. (B) Western blot shows the requirement of an intact RKKR (SEQ ID NO: 179) sequence for proteolytic generation of short (cleaved) Foxp3. Asterisk indicates endogenous Foxp3 and arrow in lane 3 indicates the short cleaved Foxp3 not detected in the sample corresponding to QNKS (SEQ ID NO: 182) --- mutant lacking both arginine (R) residues (lane 5). Abbreviations: C, cytoplasmic; N, nuclear; T, total extracts; chro-b, chromatin-bound fraction. (Above gel photograph, SEQ ID NOs: 179, 179, 181, 182, 179, 179). (Below gel photograph, SEQ ID NOs: 179, 181, 182).

FIG. 9. Retroviral expression and subcellular localization of WT-Foxp3 and Foxp3 mutants in CD4+ T cells 3 days following retroviral infection. (A) Expression of WT-Foxp3 and the Foxp3 mutants RKKR• (SEQ ID NO: 179), QNKR-(SEQ ID NO: 181), QNKR• (SEQ ID NO: 181) and empty vector Minr-1 (a control). Total extracts were prepared and analyzed by SDS-PAGE and Western blotting. Lower panel shows hNGFR levels on the same blot. (B) Foxp3 and Foxp3 mutant levels in nuclear and cytoplasmic extracts. Following determination of Foxp3 levels, the same blot was used to determine hNGFR and SP1 expression to assess the efficiency of separation of the nuclear and cytoplasmic compartments. (SEQ ID NOs: 179, 181, 181).

FIG. 10. Demonstration of proteolytic cleavage and its dependence on an intact RXXR (SEQ ID NO: 180) motif in chromatin-bound Foxp3. The Foxp3 constructs are shown schematically above the Western blot (numbers 1 through 8). Mutant constructs and residues are highlighted. Arrowheads in WT-Foxp3 indicate the location of cleavage sites.

Sample 2 was used as size control. (First row, SEQ ID NOs: 180, 183; Shaded oval, SEQ ID NOs: 183, 179; First column of table, top to bottom, SEQ ID NOs: 191, 193, 193, 191; Second column of table, SEQ ID NO: 179).

FIG. 11. (A) Proteolytic cleavage of chromatin-bound Foxp3 at 48RDLR↓S52 (SEQ ID NO: 191). Foxp3 mutants are shown schematically above the Western blot (sample 1 is WT-Foxp3). N, nuclear extract; C, cytoplasmic extract; chr, chromatin. Arrowheads in WT-Foxp3 diagram (sample1) show the cleavage sites. Mutant constructs and residues are highlighted. (Shaded oval, SEQ ID NOs: 183, 179; Second column, SEQ ID NOs: 191, 191, 193; Fourth column, SEQ ID No: 179). (B) Western blot of a chromatin extract from CD4+ cells retrovirally expressing WT-Foxp3. Antibodies are NRRF-30 mAb (ebioscience), and FJK-16s mAb (ebioscience).

FIG. 12. Activation of mouse natural Tregs (CD4+CD25+ population) and the generation of the 41-kDa Foxp3 species. Double-cleaved (N- and C-terminal) 41 kDa Foxp3 is detectable only in activated natural Tregs in the chromatin-bound fraction. Natural Tregs harvested from spleen and lymph nodes were purified over MACS columns. Foxp3 expression in different cellular fractions were analyzed by Western blotting (unless otherwise stated). Lanes 1-3, Nuclear, cytoplasmic extracts and the chromatin fraction (N, C, chr) of resting (non-activated) natural Tregs; Lanes 4-6, same fractions but after activation overnight on plates previously coated with α-CD3 and α-CD28 (2 μg/ml final conc.); lanes 7-8, nuclear extracts, same as lanes 1 and 4 but ten times more loaded; lanes 9 and 10, chromatin fraction, same as lanes 3 and 6 but ten times more loaded; lanes 11-16, controls lanes, half of all the initial samples were saved and instead of being prepared for Western blotting, they were extracted with 0.2M H₂SO₄ and analyzed for histone content on an acetic acid-urea gel. Detection of histones was by direct staining of the gel with Amido-black. Lanes 17-19, Nuclear, cytoplasmic extracts and the chromatin fraction from natural activated Tregs, activated overnight with PMA (3 ng/ml) and ionomycin (2 μM). The arrows mark the 41-kDa Foxp3 species.

FIG. 13. Suppression of Teff cell proliferation by different Foxp3 forms. WTFoxp3: M1---RDLRS52--------RKKRS418--P429 (429-aa) (SEQ ID NOs: 191, 188); N-cleaved Foxp3: M1S52--------RKKRS418--P429 (378-aa) (SEQ ID NO: 188); C-cleaved Foxp3: M1---RDLRS52--------RKKR417• (417-aa) (SEQ ID NO: 188, 179); N-plus C-cleaved Foxp3: M1S52--------RKKR417• (366-aa) (SEQ ID NO: 179). In the C-cleaved PNNW (SEQ ID NO: 184) mutant, RKKR (SEQ ID NO: 179) sequence is replaced with PNNW (SEQ ID NO: 184) and has the structure: M1---RDLRS52--------PNNW417• (417-aa). (SEQ ID NOs: 191, 184). (•) indicates engineered C-terminal ends (stop codons). Data is representative of several experiments. Transduced cells expressing empty Minr-1 vector suppress Teff cell proliferation similar to C-cleaved (PNNW) (SEQ ID NO: 184) mutant.

FIG. 14. Effect of Foxp3 mutations in vivo using a murine IBD model. RAG−/− mice (5/group) were co-injected with 1×106 CD4+CD25− and 1×105 CD4+ transduced T cells (10:1 ratio) expressing the WT-Foxp3, Foxp3 mutants RKKR. (SEQ ID NO: 179), QNKR• (SEQ ID NO: 181), QNKR-- (SEQ ID NO: 181) or control Minr-1 vector. (A) Serial analysis of weight loss showed the benefit WT-Foxp3 versus MINR1 or no Tregs (p<0.05), whereas RKKR• (SEQ ID NO: 179) was significantly more suppressive than WT-Foxp3 (p<0.05). (B) Comparison of events within duodenal samples collected at day 45. Injection of cells expressing WT-Foxp3 was associated with mild mononuclear cell recruitment and villous edema, whereas animals receiving cells expressing short-Foxp3 (RKKR•) (SEQ ID NO: 179) had essentially normal histology (H&E-stained paraffin sections, original magnifications ×200). Immunoperoxidase staining showed infiltration by Foxp3+ mononuclear cells in both cases (hematoxylin-counterstained cryostat sections, original magnifications ×400, inset shows lack of staining using isotype-matched control mAb). (C) Use of cells expressing short Foxp3 (QNKR•) (SEQ ID NO: 181) was significantly more effective in controlling weight loss in this model than use of cells expressing long-Foxp3 (QNKR--) (SEQ ID NO: 181) (p<0.05). (D) Histology of duodenal samples showed minor mononuclear cell infiltration using QNKR• (SEQ ID NO: 181) but extensive mononuclear cell recruitment in the case of mice receiving QNKR-- (SEQ ID NO: 181), and corresponding immunoperoxidase analysis showed Foxp3+ cells in both groups (inset shows lack of staining using an isotype control mAb, details as for panel b). (E) Absolute cell numbers harvested from the spleens and mesenteric lymph nodes of each group; the short Foxp3 (RKKR•) (SEQ ID NO: 179) group yielded the least number of total splenic T cells (*p<0.05), consistent with the absence of inflammatory disease in this group, whereas the small numbers of mesenteric LN T cells were not statistically significant between groups. (Sequences along bottom edge of table: SEQ ID NOs: 179, 181, 181).

FIG. 15. Proposed mechanism of biochemical activation of Foxp3. (SEQ ID NOs: 179, 183).

### DETAILED DESCRIPTION OF THE INVENTION

The generation of functional Tregs is important for suppressing aberrant activation of the immune system and maintaining immune system homeostasis and self-tolerance, as loss of this regulation is associated with autoimmune diseases, and transplant rejection (i.e., graft vs. host disease, GVHD). Elucidation of the molecular mechanism responsible for the development of functional Tregs facilitates the development of compositions and methods for manipulating the genetic program that specifies this cell fate. Additionally, such compositions and methods could be used to advantage to detect Treg activity and function. Alternatively, it is desirable to inhibit Treg formation in cancer. The present invention provides screening assays to identify agents which modulate Treg function and activity.

The present invention is based, at least in part, on the finding that certain molecules are preferentially associated with effector T cells or regulatory T cells. Accordingly, immune responses by one or the other subset of cells can be preferentially modulated. The invention pertains, e.g., to methods of modulating (e.g., up- or down-modulating), the balance between the activation of regulatory T cells and effector T cells leading to modulation of immune responses and to compositions useful in modulating those responses. The invention also pertains to methods useful in diagnosing, treating, or preventing conditions that would benefit from modulating effector T cell function relative to regulatory T cell function or from modulating regulatory T cell function relative to effector T cell function in a subject. Previous studies have defined three distinct nuclear localization signals (NLSs) in Foxp3, each of which is sufficient to mediate nuclear import of the protein. One of these NLSs is located at the N-terminal of the forkhead domain (NLS1), and another is located in the last 12 amino acids at the C-terminus of Foxp3 (NLS2). A third motif in the forkhead domain (NLS3), is a conditional NLS and only functions upon removal of the 12 amino acids at the C-terminus of the protein.

Different Foxp family members share conserved structural domains, and the amino acids 416-417 (KR) are involved in Foxp3 DNA binding (25, 29). The data contained herein demonstrates that amino acids 414-417 (RKKR) (SEQ ID NO: 179) are masked by the C-terminal end of the molecule, and that Foxp3 undergoes a conformational change which exposes this regulatory region of the protein. The conformational change can be brought about through proteolytic cleavage which releases the C-terminus and exposes NLS3. 414RKKR417 (SEQ ID NO: 179) represents an ideal recognition sequence for members of the pro-protein convertase (PC) family, which appear to be responsible for cleavage of Foxp3. Indeed, elaboration of the C-terminal cleavage product (termed “Foxpeptide”—12aa in mouse, and 14aa in human) provides an indicator of Treg activity. Also, as described hereinbelow, engineered human short-Foxp3, which is 14 amino acids shorter at the C-terminal end, mimics a proteolytically cleaved Foxp3 and has utility in the treatment of autoimmune diseases and the treatment of transplantation patients. Alternatively, reducing Treg activity or inhibiting the generation of functional Tregs could be useful for the treatment of cancer. Elaboration of the peptide also provides means to monitor Treg activity.

In one embodiment, a method for identifying agents that affect the formation of double-cleaved Foxp3 is provided. Exemplary methods entail the use of cell lines and/or whole transgenic animal models wherein Foxp3 is over or under expressed. The agents identified preferably modulate the activity of the proprotein convertase enzymes that process Foxp3 into its short form. Agents identified using the methods of the invention may be used alone for the treatment of a particular disorder, or may be combined with other agents known to have efficacy for the treatment of the particular disorder. The agents can be useful for treating patients with autoimmune disease (i.e. the agent will stimulate proprotein convertase activity) or cancer (i.e., the agent will inhibit proprotein convertase activity).

In another embodiment, isolated nucleic acids encoding the C-terminal cleaved portion of Foxp3 (Foxpeptide) and means to detect the same are provided to facilitate the detection of activated Tregs. Other nucleic acids are also provided which encode short forms of Foxp3 which can be used therapeutically.

As discussed herein, Foxp3 plays a major role in regulatory T cell development, and the gene knock-out phenotype is characterized by multi-organ inflammatory response, lack of CD4+CD25+ Treg cells, T-cells with an activated phenotype, eosinophilia, dysregulated cytokine production, hyperimmuneglobulinemia, and males dying at 3 weeks.

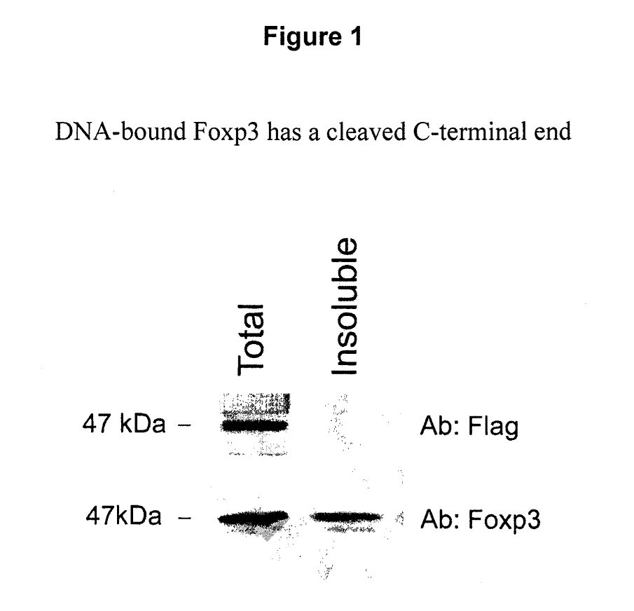

In yet another embodiment, a novel Foxpeptide is disclosed which can be detected in a patient. In particular, the construct consists of amino acids 418-431 of human Foxp3 (Foxpeptide) which has the sequence of SEQ ID NO: 8. See FIG. 1D. To detect C-terminal cleaved Foxp3, a biological sample is provided and assessed for the presence of Foxpeptide which indicates Tregs are active in the system.

For therapeutic use, the compositions of the invention may be administered in any conventional dosage form in any conventional manner. Routes of administration include, but are not limited to, intravenous, intramuscular, subcutaneous, intrasynovial, infusion, sublingual, transdermal, oral, topical, aerosolized, or inhalation via a nebulizer.

The following description and examples set forth the general procedures involved in practicing the present invention. To the extent that specific materials are mentioned, it is merely for purposes of illustration and is not intended to limit the invention. Unless otherwise specified, general biochemical and molecular biological procedures, such as those set forth in Sambrook et al., Molecular Cloning, Cold Spring Harbor Laboratory (1989) (hereinafter “Sambrook et al.”) or Ausubel et al. (eds.) Current Protocols in Molecular Biology, John Wiley & Sons (1997) (hereinafter “Ausubel et al.”) are used.

### I. Definitions:

The following definitions are provided to facilitate an understanding of the present invention:

As used herein, the term “regulatory T cell”, “suppressor T cell”, or “Treg” includes T cells which produce low levels of IL-2, IL-4, IL-5, and IL-1, and actsto suppress activation of the immune system. Regulatory T cells actively suppress the proliferation and cytokine production of Th1, Th2, or naive T cells which have been stimulated in culture with an activating signal (e.g., antigen and antigen presenting cells or with a signal that mimics antigen in the context of MHC, e.g., anti-CD3 antibody, plus anti-CD28 antibody).

As used herein, the term “effector T cell”, or “Teff”includes T cells which function to eliminate antigen (e.g., by producing cytokines which modulate the activation of other cells or by cytotoxic activity). The term “effector T cell” includes T helper cells (e.g., Th1 and Th2 cells) and cytotoxic T cells. Th1 cells mediate delayed type hypersensitivity responses and macrophage activation while Th2 cells provide help to B cells and are critical in the allergic response.

The phrase “Foxpeptide” as used herein refers to the C-terminal fragment that is a cleavage product from Foxp3 (12-amino acids in mouse Foxp3 coded for by SEQ ID NO: 5 and 14-amino acids in human Foxp3 coded for by SEQ ID NO: 7.

The phrase “short Foxp3” as used herein refers to amino acids 1-417 of Foxp3 which results from the enzymatic processing of the C-terminus of Foxp3. This construct is functional in preventing effector T cell proliferation and is effective to prevent endogenous IL-2 expression.

The phrase “double cleaved Foxp3” or “double cleaved short Foxp3” as used herein refers the remaining portion of Foxp3 following cleavage at both the N- and C-terminus (resulting in a polypeptide of amino acids 52-417 of the full length Foxp3). This construct has therapeutic applications when delivered to a patient.

A “proprotein convertase” includes without limitation, calcium dependent subtilisin/kexin-related serine endopeptidases. Examples are Furin, PC1/3, PC2, PC4, PACE4, PC5/6. and PC7. Proprotein convertase inhibitors have been described previously and include, for example, polybasic peptides such as L-poly-Arg (for Furin), and the short hexapeptide of the sequence LLRVKR (SEQ ID NO: 185) (for PC1). See Furgure et al. Molecular Pharmacology 71:323-332 (2007); Cameron et al. J. Biol. Chem. 275:36741-36749 (2000); Fugere et al, Curr. Pharm. Design 8:125-133 (2002). Proprotein convertase inhibitors identified using the methods of the invention do not include the peptides described above.

As used herein the phrase, “modulating regulatory T cell function” includes preferentially altering at least one regulatory T cell function (in a population of cells including both T effector cells and T regulatory cells) such that there is a shift in the balance of T effector/T regulatory cell activity as compared to the balance prior to treatment.

An “autoimmune disease” as used herein refers to a disease associated with the inability of the immune system to discriminate between self and non-self. Examples of autoimmune diseases include, without limitation, immune dysregulation, polyendocrinopathy, enteropathy, X-linked (IPEX), type 1 diabetes, arthritis (including rheumatoid arthritis, juvenile rheumatoid arthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, iritis, conjunctivitis, keratoconjunctivitis, ulcerative colitis, asthma, allergic asthma, cutaneous lupus erythematosus, scleroderma, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves ophthalmopathy, sarcoidosis, primary biliary cirrhosis, uveitis posterior, and interstitial lung fibrosis.

The phrase “immunomonitoring” as used herein refers to detecting the presence of Foxpeptide in a biological sample as an indication of active Tregs in a system.

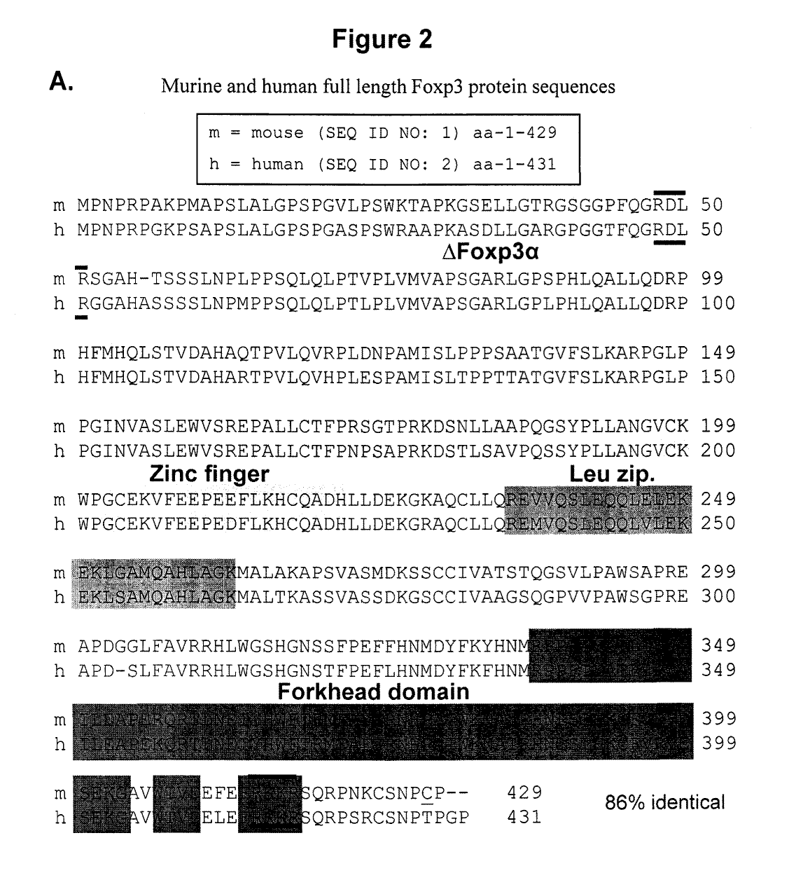

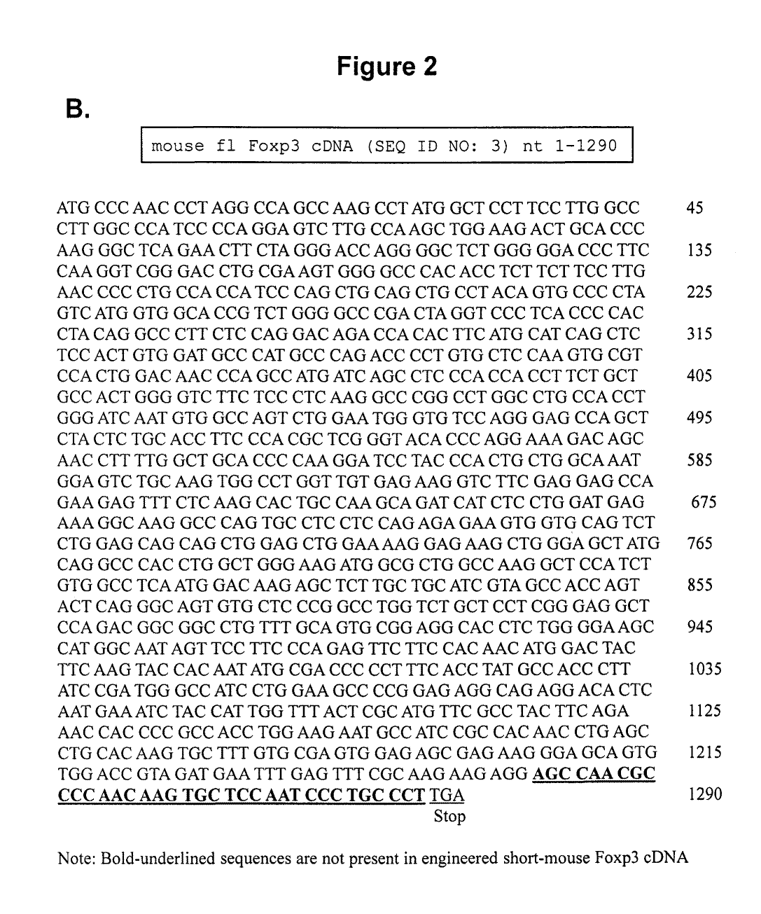

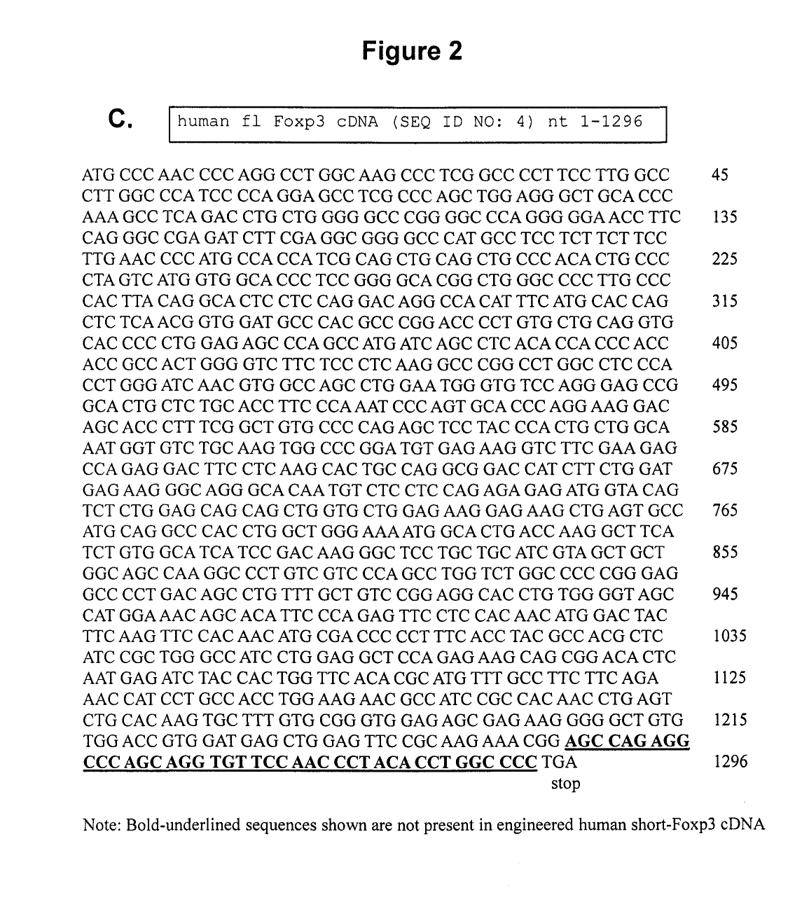

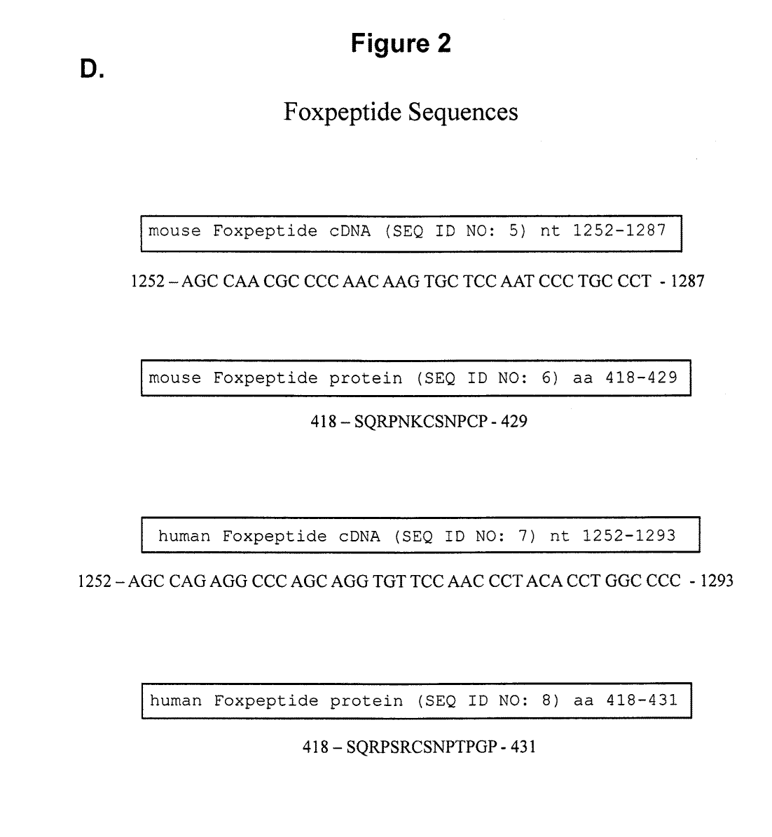

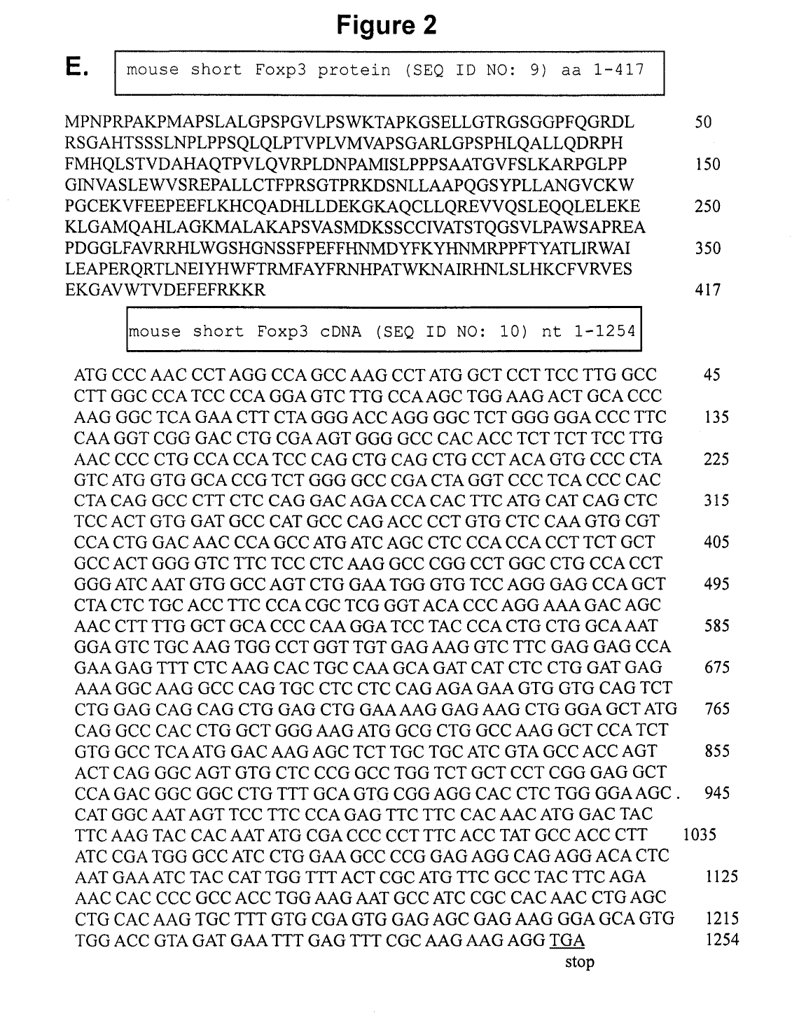

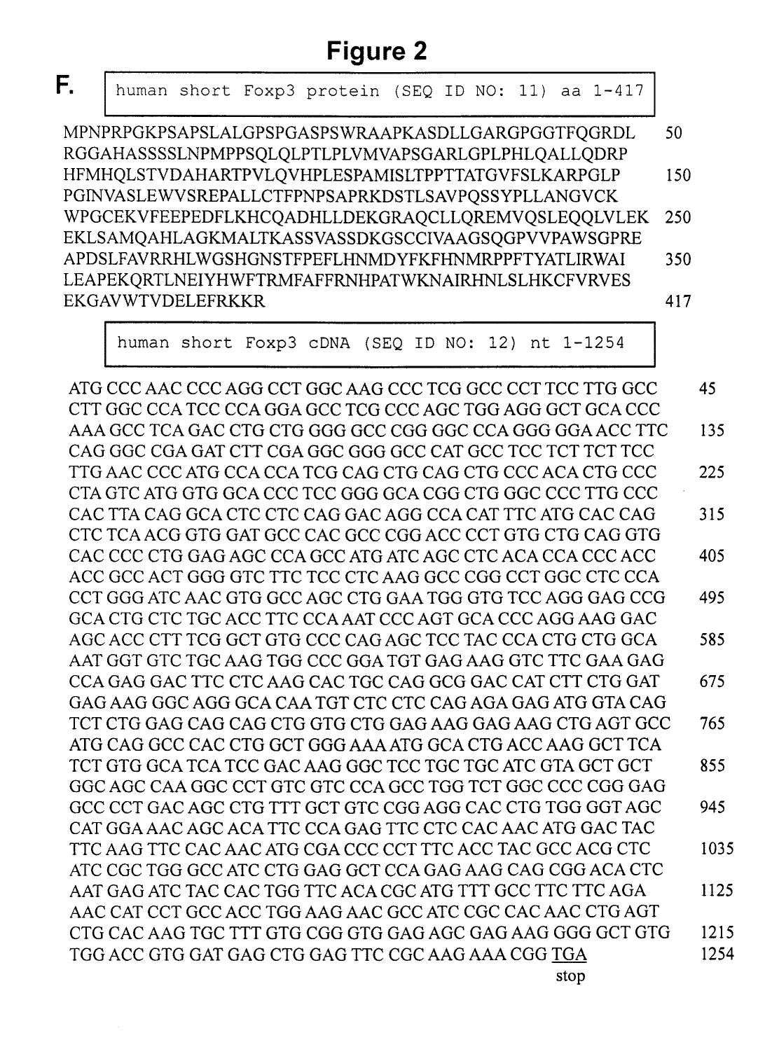

“Sample” or “patient sample” or “biological sample” as used herein generally refers to a sample which may be tested for the presence or absence of a particular molecule, preferably Foxpeptide, as shown in FIG. 2D. Samples may include but are not limited to cells, including tissue, and body fluids including blood, serum, plasma, urine, saliva, tears, pleural fluid and the like.

As used herein, the phrase “treating an autoimmune disease” is intended to refer to the alleviation of a sign or symptom of an autoimmune disease. Treating an autoimmune disease is intended to encompass a reduction in the onset or magnitude of a sign or symptom of an autoimmune disease, such as effector T cell proliferation.

As used herein, “treating cancer” refers to modulating T cells to shift the balance to Teff cell function relative to Treg function.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

With respect to single-stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (see Sambrook et al. (2001) Molecular Cloning. A Laboratory Manual, Cold Spring Harbor, N.Y.: Cold Spring Harbor Laboratory Press):

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. Depending upon the specific sequence involved, the Tₘ of a DNA duplex decreases by 0.5-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5× Denhardt's solution, 0.5% SDS and 100 μg/ml denatured salmon sperm DNA at 42° C., and washed in 2×SSC and 0.5% SDS at 55° C. for 15 minutes. A high-stringency hybridization is defined as hybridization in 6×SSC, 5× Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5× Denhardt's solution, 0.5% SDS and 100 μg/ml denatured salmon sperm DNA at 42° C., and washed in 0.1×SSC and 0.5% SDS at 65° C. for 15 minutes.

The present invention also includes active portions, fragments, derivatives and functional or non-functional mimetics of the polypeptides of the invention. “Peptide” and “polypeptide” are used interchangeably herein and refer to a compound made up of a chain of amino acid residues linked by peptide bonds. An “active portion” of a polypeptide means a peptide that is less than the full length polypeptide, but which retains measurable biological activity and retains biological detection.

A “fragment” or “portion” of a polypeptide means a stretch of amino acid residues of at least about five to seven contiguous amino acids, often at least about seven to nine contiguous amino acids, typically at least about nine to fifteen contiguous amino acids and, most preferably, at least about fourteen or more contiguous amino acids.

A “derivative” of a polypeptide or a fragment thereof means a polypeptide modified by varying the amino acid sequence of the protein, e.g. by manipulation of the nucleic acid encoding the protein or by altering the protein itself Such derivatives of the natural amino acid sequence may involve insertion, addition, deletion or substitution of one or more amino acids, and may or may not alter the essential activity of original the polypeptide.

As mentioned above, a polypeptide or protein of the invention includes any analogue, fragment, derivative or mutant which is derived from a polypeptide and which retains at least one property or other characteristic of the polypeptide. Different “variants” of the polypeptide exist in nature. These variants may be alleles characterized by differences in the nucleotide sequences of the gene coding for the protein, or may involve different RNA processing or post translational modifications. The skilled person can produce variants having single or multiple amino acid substitutions, deletions, additions or replacements. These variants may include inter alia: (a) variants in which one or more amino acids residues are substituted with conservative or non conservative amino acids, (b) variants in which one or more amino acids are added to the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Other polypeptides of the invention include variants in which amino acid residues from one species are substituted for the corresponding residue in another species, either at the conserved or non conserved positions. In another embodiment, amino acid residues at non conserved positions are substituted with conservative or non conservative residues. The techniques for obtaining these variants, including genetic (suppressions, deletions, mutations, etc.), chemical, and enzymatic techniques are known to the person having ordinary skill in the art.

To the extent such allelic variations, analogues, fragments, derivatives, mutants, and modifications, including alternative nucleic acid processing forms and alternative post translational modification forms result in derivatives of the polypeptide that retain any of the biological properties of the polypeptide, they are included within the scope of this invention.

The term “functional” as used herein implies that the nucleic or amino acid sequence is functional for the recited assay or purpose.

The term “tag,” “tag sequence” or “protein tag” refers to a chemical moiety, either a nucleotide, oligonucleotide,polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

Many techniques are available to those skilled in the art to facilitate transformation, transfection, or transduction of the expression construct into a prokaryotic or eukaryotic organism. The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “gene” refers to a nucleic acid comprising an open reading frame encoding a polypeptide, including both exon and (optionally) intron sequences. The nucleic acid may also optionally include non coding sequences such as promoter or enhancer sequences. The term “intron” refers to a DNA sequence present in a given gene that is not translated into protein and is generally found between exons.

The term “promoter” or “promoter region” generally refers to the transcriptional regulatory regions of a gene. The “promoter region” may be found at the 5′ or 3′ side of the coding region, or within the coding region, or within introns. Typically, the “promoter region” is a nucleic acid sequence which is usually found upstream (5′) to a coding sequence and which directs transcription of the nucleic acid sequence into mRNA. The “promoter region” typically provides a recognition site for RNA polymerase and the other factors necessary for proper initiation of transcription.

The term “vector” relates to a single or double stranded circular nucleic acid molecule that can be infected, transfected or transformed into cells and replicate independently or within the host cell genome. A circular double stranded nucleic acid molecule can be cut and thereby linearized upon treatment with restriction enzymes. An assortment of vectors, restriction enzymes, and the knowledge of the nucleotide sequences that are targeted by restriction enzymes are readily available to those skilled in the art, and include any replicon, such as a plasmid, cosmid, bacmid, phage or virus, to which another genetic sequence or element (either DNA or RNA) may be attached so as to bring about the replication of the attached sequence or element. A nucleic acid molecule of the invention can be inserted into a vector by cutting the vector with restriction enzymes and ligating the two pieces together.

The terms “recombinant organism,” or “transgenic organism” refer to organisms which have a new combination of genes or nucleic acid molecules. A new combination of genes or nucleic acid molecules can be introduced into an organism using a wide array of nucleic acid manipulation techniques available to those skilled in the art. The term “organism” relates to any living being comprised of a least one cell. An organism can be as simple as one eukaryotic cell or as complex as a mammal. Therefore, the phrase “a recombinant organism” encompasses a recombinant cell, as well as eukaryotic and prokaryotic organisms.

The alteration or genetic information may be foreign to the species of organism to which the recipient belongs, or foreign only to the particular individual recipient, or may be genetic information already possessed by the recipient. In the last case, the altered or introduced gene may be expressed differently than the native gene. The altered Foxp3 gene generally should not fully encode the same Foxp3 protein native to the host animal and its expression product should be altered to a minor or great degree.

The DNA used for altering a target gene may be obtained by a wide variety of techniques that include, but are not limited to, isolation from genomic sources, preparation of cDNAs from isolated mRNA templates, direct synthesis, or a combination thereof.

As used herein, the term “agent” includes compounds that modulate, e.g., up-modulate or stimulate and down-modulate or inhibit, the expression and/or activity of a molecule of the invention. As used herein the term “inhibitor” or “inhibitory agent” includes agents which inhibit the expression and/or activity of a molecule of the invention. Exemplary inhibitors include antibodies, RNAi, compounds that mediate RNAi (e.g., siRNA), antisense RNA or DNA, dominant/negative mutants of molecules of the invention, peptides, and/or peptidomimetics.

The term “stimulator” or “stimulatory agent” includes agents, e.g., agonists, which increase the expression and/or activity of molecules of the invention. Exemplary stimulating agents include active protein and nucleic acid molecules, peptides and peptidomimetics of molecules of the invention. The agents of the invention can directly modulate, i.e., increase or decrease, the expression and/or activity of a molecule of the invention. Exemplary agents are described herein or can be identified using screening assays that select for such compounds, as described in detail below.

An “siRNA” or “small interfering RNA” refers to a molecule involved in the RNA interference process for a sequence-specific post-transcriptional gene silencing or gene knockdown. SiRNAs have homology with the sequence of the targeted gene. SiRNAs can be synthesized in vitro or generated by ribonuclease III cleavage from longer dsRNA and are the mediators of sequence-specific mRNA degradation. Preferably, the siRNA of the invention are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Applied Biosystems (Foster City, Calif., USA), Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK). Specific siRNA constructs for inhibiting proprotein convertase enzyme mRNA, and may be between 15-35 nucleotides in length, and more typically about 21 nucleotides in length. A list of candidate siRNAs for PC1, PC7 and Furin are provided in Table I-III, and are useful for the treatment of cancer by decreasing Tregs activity in a patient, and allowing Teffs to target tumor cells.

As used herein, the term “pharmaceutically-acceptable carrier” means a chemical composition with which an appropriate siRNA, may be combined and which, following the combination, can be used to administer the siRNA to a patient. The amount of the siRNA composition administered is sufficient to prevent, diminish or alleviate the disease state. The pharmaceutical compositions useful for practicing the invention may be administered to deliver a dose of between about 1 ng/kg and about 100 mg/kg of patient body weight. Suitable amounts of the siRNA for administration include doses which are high enough to have the desired effect without concomitant adverse effects.

As used herein, the term “administration” refers to the methods of delivery of the compounds of the invention (e.g., routes of administration such as, without limitation, intravenous, intramuscular, subcutaneous, intrasynovial, infusion, sublingual, transdermal, oral, topical, aerosolized, or inhalation via a nebulizer).

Administration of proprotein convertase siRNAs by inhalation is a means of treating an individual having cancer, particularly in the lung. One skilled in the art would recognize that siRNA can be suspended or dissolved in an appropriate pharmaceutically acceptable carrier and administered, for example, directly into the lungs using a nasal spray or inhalant. A pharmaceutical composition comprising proprotein convertase siRNA can be administered as an aerosol formulation which contains the inhibitor in dissolved, suspended or emulsified form in a propellant or a mixture of solvent and propellant. The aerosolized formulation is then administered through the respiratory system or nasal passages. Methods for pulmonary delivery are described in, for example US Patent Application No. 20040037780, and U.S. Pat. Nos. 6,592,904; 6,582,728; and 6,565,885, all incorporated by reference herein.

As used herein, an “instructional material” includes a publication, a recording, a diagram, or any other medium of expression which can be used to communicate the usefulness of the composition of the invention for performing a method of the invention. The instructional material of a kit of the invention can, for example, be affixed to a container which contains a kit of the invention to be shipped together with a container which contains the kit. Alternatively, the instructional material can be shipped separately from the container with the intention that the instructional material and kit be used cooperatively by the recipient.

### II. Modulators of Foxp3

### A. Stimulatory Agents

According to a modulatory method of the invention, expression and/or activity of Foxp3 is stimulated in a cell by contacting the cell with a stimulatory agent. Examples of such stimulatory agents include active protein and nucleic acid molecules that are introduced into the cell to increase expression and/or activity of Foxp3 in the cell. Preferably, this stimulation facilitates increased activity of the enzymes that cleave and process Foxp3 into singly cleaved or double cleaved short Foxp3 forms. The administration of such agents should provide therapeutic benefit to patients suffering from autoimmune diseases.

Other stimulatory agents that can be used to stimulate the activity of a molecule of the invention are chemical compounds that stimulate expression or activity of a molecule of the invention in cells, such as compounds that directly stimulate the protein product of a molecule of the invention and compounds that promote the interaction between a protein product of a molecule of the invention and substrates or target DNA binding sites. Such compounds can be identified using screening assays that select for such compounds, as described herein.

### B. Inhibitory Agents

Inhibitory agents of the invention can be, for example, intracellular binding molecules that act to inhibit the expression or activity of a molecule of the invention. For molecules that are expressed intracellularly, intracellular binding molecules can be used to modulate expression and/or activity. As used herein, the term “intracellular binding molecule” is intended to include molecules that act intracellularly to inhibit the expression or activity of a protein (i.e., Foxp3) by binding to the protein itself, to a nucleic acid (e.g., an mRNA molecule) that encodes the protein or to a target with which the protein normally interacts (e.g., to a DNA target sequence to which the marker binds). Examples of intracellular binding molecules include antisense marker nucleic acid molecules (e.g., siRNA against a Foxp3 processing enzyme to inhibit translation of mRNA), intracellular antibodies (e.g., to inhibit the activity of protein) and dominant negative mutants.

Several classes of compound may be used according to the invention as convertase inhibitors. These compounds include: (1) compounds that bind to convertase enzymes and inhibit its activity (e.g. competitive inhibitors or allosteric inhibitors); (2) compounds which prevent the transcription, translation or expression of convertase enzymes (e.g. ribozymes, siRNA, or antisense DNA molecules); (3) compounds which increase the rate of degradation of convertase enzymes; (4) compounds which inhibit the interaction of convertase enzymes with latent TGF-beta; (5) compounds which inhibit the proteolytic activation of the inactive Furin or other PC precursors; and (6) compounds which inhibit a potential intracellular translocation of convertase enzymes, such as Furin or PACE-4, to subcellular sites of activity.

In one embodiment of the invention, a composition containing proprotein convertase siRNA is administered to a patient in a sufficient amount to prevent, diminish or alleviate a cancerous state in the individual by affecting Foxp3 processing and Treg formation. There are several ways to administer the siRNA of the invention to in vivo to treat cancer including, but not limited to, naked siRNA delivery, siRNA conjugation and delivery, liposome carrier-mediated delivery, polymer carrier delivery, nanoparticle compositions, plasmid-based methods, and the use of viruses.

siRNA composition of the invention can comprise a delivery vehicle, including liposomes, for administration to a subject, carriers and diluents and their salts, and/or can be present in pharmaceutically acceptable formulations. This can be necessary to allow the siRNA to cross the cell membrane and escape degradation. Methods for the delivery of nucleic acid molecules are described in Akhtar et al., 1992, Trends Cell Bio., 2, 139; Delivery Strategies for Antisense Oligonucleotide Therapeutics, ed. Akhtar, 1995, Maurer et al., 1999, Mol. Membr. Biol., 16, 129-140; Hofland and Huang, 1999, Handb. Exp. Pharmacol., 137, 165-192; and Lee et al., 2000, ACS Symp. Ser., 752, 184-192; Beigelman et al., U.S. Pat. No. 6,395,713 and Sullivan et al., PCT WO 94/02595 further describe the general methods for delivery of nucleic acid molecules.

As mentioned previously, one embodiment of the invention comprises delivery of the proprotein convertase siRNA to a patient in need thereof. In other embodiments, polybasic peptides can be used to inhibit Furin as described in U.S. Pat. No. 7,0337,991. Alpha-PDX can also be used as described in Anderson et al. (J. Biol. Chem. (1993) 268: 24887-24891). Candidate siRNA compositions for use in the invention are provided in Tables I-III. The sequences in Tables I-III include several siRNAs (i.e., sense strands are provided for a proprotein convertase target region). Those of skill in the art can determine the sequence of an antisense siRNA strand based on the disclosure of the sense strand, and will appreciate the difference between “U” and “T” designations in the sequences which correspond to RNA and DNA molecules, respectively.

TABLE I
Candidate PC1 siRNA molecules (sense strands)
based on GenBank Accession NM_000439

| | |
|---|---|
| SEQ ID NO: 25 | CCTGAAAGCTAATGGAGAAtt |
| SEQ ID NO: 26 | CAATAACCCTGGATGGAAAtt |
| SEQ ID NO: 27 | CCCTAATGATGATGGGAAAtt |
| SEQ ID NO: 28 | GGAGAGAACCCTATAGGTAtt |
| SEQ ID NO: 29 | GCTGAAAGAGAACGGGATAtt |
| SEQ ID NO: 30 | TGATGATGATCGTGTGATAtt |
| SEQ ID NO: 31 | ACTTGGACTTTGAGAATTAtt |
| SEQ ID NO: 32 | AGAAAGAGTGTGTTGTAAAtt |
| SEQ ID NO: 33 | AGAAAGAAGTAAACGTTCAtt |
| SEQ ID NO: 34 | AAGTAAACGTTCAGCTCTAtt |
| SEQ ID NO: 35 | CCACAAACGAGAACAAACAtt |
| SEQ ID NO: 36 | GACCAGATGTGCAGGAGAAtt |
| SEQ ID NO: 37 | CCAAAGCTCTGGTGGATTTtt |
| SEQ ID NO: 38 | CAAGAGAACCCTAAGGAGAtt |
| SEQ ID NO: 39 | GAACAGTGCAAAAGCGAAAtt |
| SEQ ID NO: 40 | CAATGGTACTTGCAAGATAtt |
| SEQ ID NO: 41 | CAGATGTGCAGGAGAAATTtt |
| SEQ ID NO: 42 | CCAGAAGGCTTTTGAATATtt |
| SEQ ID NO: 43 | GTGGATATTTACAGTGCAAtt |
| SEQ ID NO: 44 | GAATAGTCGATTTGGATTTtt |
| SEQ ID NO: 45 | GGAGCGTGCCTGAGAAGAAtt |
| SEQ ID NO: 46 | GAAGAAAGAGTGTGTTGTAtt |
| SEQ ID NO: 47 | GTACTTGGACTTTGAGAATtt |
| SEQ ID NO: 48 | AAGAGAACCCTAAGGAGAAtt |
| SEQ ID NO: 49 | GGAGCAGGCTTGATGGTGAtt |
| SEQ ID NO: 50 | CCTAATGGCTTTAAGAACTtt |
| SEQ ID NO: 51 | GAACAAACCTTCCCAGCTTtt |
| SEQ ID NO: 52 | TGTGATACCTGTTTGGCAAtt |
| SEQ ID NO: 53 | GCAAGCAAATAATCACAAAtt |
| SEQ ID NO: 54 | GAGCAGGCTTGATGGTGAAtt |
| SEQ ID NO: 55 | GGGCTGAACAACAGTATGAtt |
| SEQ ID NO: 56 | GGGCAAAGGAGTTGTTATCtt |
| SEQ ID NO: 57 | GAAGAGGGGTGGAGAAGATtt |
| SEQ ID NO: 58 | CAGAAGGTCTCGAAGGAGTtt |
| SEQ ID NO: 59 | GCTGAACAACAGTATGAAAtt |
| SEQ ID NO: 60 | GGAATCACACGGACATTTAtt |
| SEQ ID NO: 61 | GGTTGGAGTTGCATACAATtt |
| SEQ ID NO: 62 | GGGAGATAATTGTGACTGTtt |
| SEQ ID NO: 63 | GGAGAAGTTATCATTGAAAtt |
| SEQ ID NO: 64 | CATTGAAATTCCAACAAGAtt |
| SEQ ID NO: 65 | GCAAAAGCGAAAAGGCAATtt |
| SEQ ID NO: 66 | TGATATGGGCTGAACAACAtt |
| SEQ ID NO: 67 | CTGGACACGTGGATATTTAtt |
| SEQ ID NO: 68 | GCGCTGACCTGCACAATGAtt |
| SEQ ID NO: 69 | GAAAGCTAATGGAGAAGTTtt |
| SEQ ID NO: 70 | TCTAAGGGACTCAGCACTAtt |
| SEQ ID NO: 71 | CACTAAATCTCTTCAATGAtt |
| SEQ ID NO: 72 | CACAATGACTGCACGGAGAtt |
| SEQ ID NO: 73 | TGTACAATTTGAAGCAACAtt |
| SEQ ID NO: 74 | CCACACAAGAGAACCCTAAtt |

TABLE II
Candidate PC7 siRNA molecules (sense strands)
based on GenBank Accession NM_004716

| | |
|---|---|
| SEQ ID NO: 75 | AGACAAAGGCTGTTAGAGAtt |
| SEQ ID NO: 76 | CAGCAAGGATCCAGACGAAtt |
| SEQ ID NO: 77 | GGAGGAAGGGACAGAGCTAtt |
| SEQ ID NO: 78 | CCATAGGAGCTGTGGATGAtt |
| SEQ ID NO: 79 | TGGAAAGCCTGGAAGGTGAtt |
| SEQ ID NO: 80 | ACTACATGCTGGAAGTATAtt |
| SEQ ID NO: 81 | GGACAGAGCTAGAATCAGTtt |
| SEQ ID NO: 82 | AGCAATGGCACCTGAATAAtt |
| SEQ ID NO: 83 | CGGTGGTGGTAGTGGATGAtt |
| SEQ ID NO: 84 | CAGACGAAGTGGAAACAGAtt |
| SEQ ID NO: 85 | CAGCAATGGCACCTGAATAtt |
| SEQ ID NO: 86 | GAAAATACCTGCACGATGAtt |
| SEQ ID NO: 87 | GCAGTAGACATCAGGGACAtt |
| SEQ ID NO: 88 | CAATCAAGTTTGTAGGAGTtt |
| SEQ ID NO: 89 | CCGGAAAGCCAAGGAGGAAtt |
| SEQ ID NO: 90 | GCTATGACCTCAACTCTAAtt |
| SEQ ID NO: 91 | GAGAGTGCCATGAGTGGAAtt |
| SEQ ID NO: 92 | GAAGTATATTTGAGCCAGAtt |
| SEQ ID NO: 93 | GCATGGAGGCAGTGGCGTTtt |
| SEQ ID NO: 94 | AGGCAGTGGCGTTCAACAAtt |
| SEQ ID NO: 95 | AGGAAGGGACAGAGCTAGAtt |
| SEQ ID NO: 96 | GACCAGATGACGATGGGAAtt |
| SEQ ID NO: 97 | GTGCAGAGTGGGTCACCAAtt |
| SEQ ID NO: 98 | GGACATTGCACCCAACTATtt |
| SEQ ID NO: 99 | CGGATGTGGAGAATGGCAAtt |
| SEQ ID NO: 100 | GCGATGTGCAGGAGAGATCtt |
| SEQ ID NO: 101 | GGGTGACGGTGGTGGTAGTtt |
| SEQ ID NO: 102 | TGGATGACGGAGTGGAACAtt |
| SEQ ID NO: 103 | CATCGGAGCCGGAAAGCCAtt |
| SEQ ID NO: 104 | GCAAGGATCCAGACGAAGTtt |
| SEQ ID NO: 105 | AGTGGATGACGGAGTGGAAtt |
| SEQ ID NO: 106 | CAGGAGAGATCGCGGCTGTtt |
| SEQ ID NO: 107 | CTACGTCAGTCCCGTGTTAtt |
| SEQ ID NO: 108 | CCCTGGAGGTCCTGTGGAAtt |
| SEQ ID NO: 109 | GGATGGACCTGGAGATGTCtt |
| SEQ ID NO: 110 | GAATGTGGCTTCCAATCAAtt |
| SEQ ID NO: 111 | ACGGGAAGGAGGAGCAGATtt |
| SEQ ID NO: 112 | CAGACAGCATGGAGGCAGTtt |
| SEQ ID NO: 113 | ACGTCAGTCCCGTGTTAAAtt |
| SEQ ID NO: 114 | GACATCAGGGACAGACAAAtt |
| SEQ ID NO: 115 | CTACATGCTGGAAGTATATtt |
| SEQ ID NO: 116 | AGCCAAGGAGGAAGGGACAtt |
| SEQ ID NO: 117 | CCAAGGAGGAAGGGACAGAtt |
| SEQ ID NO: 118 | AGGGACAGAGCTAGAATCAtt |
| SEQ ID NO: 119 | ACGCAATGTGACTGGGCGAtt |
| SEQ ID NO: 120 | GCAACGGAGGCCAACACAAtt |
| SEQ ID NO: 121 | CACCATAGGAGCTGTGGATtt |
| SEQ ID NO: 122 | GCTGAAGACCCTGGAGCATtt |
| SEQ ID NO: 123 | ACAAAGGCTGTTAGAGAGTtt |
| SEQ ID NO: 124 | TATATTTGAGCCAGAGGAAtt |

TABLE III
Candidate Furin siRNA molecules (sense strands)
Based on GenBank Accession NM_002569

| | |
|---|---|
| SEQ ID NO: 125 | GCCCAAAGACATCGGGAAAtt |
| SEQ ID NO: 126 | TGGAACAGCAGGTGGCAAAtt |
| SEQ ID NO: 127 | GGACTAAACGGGACGTGTAtt |
| SEQ ID NO: 128 | CGGCAGAAGTGCACGGAGTtt |
| SEQ ID NO: 129 | ACACACAGATGAATGACAAtt |
| SEQ ID NO: 130 | GCACTATAGCACCGAGAATtt |
| SEQ ID NO: 131 | AGAATGACGTGGAGACCATtt |
| SEQ ID NO: 132 | CGAGTGGGTCCTAGAGATTtt |
| SEQ ID NO: 133 | TGGCAAAGCGACGGACTAAtt |
| SEQ ID NO: 134 | GCAGATGGGTTTAATGACTtt |
| SEQ ID NO: 135 | GCTCAGGGCCAGAAGGTCTtt |
| SEQ ID NO: 136 | GTGGCAAAGCGACGGACTAtt |
| SEQ ID NO: 137 | CAACGGTGTCTGTGGTGTAtt |
| SEQ ID NO: 138 | GAACATGACAGCTGCAACTtt |
| SEQ ID NO: 139 | CCAGCGAAGCCAACAACTAtt |
| SEQ ID NO: 140 | ACTATAGCACCGAGAATGAtt |
| SEQ ID NO: 141 | GGTACACACAGATGAATGAtt |
| SEQ ID NO: 142 | GCAACCAGAATGAGAAGCAtt |
| SEQ ID NO: 143 | CGGAAGTGCATCATCGACAtt |
| SEQ ID NO: 144 | GCGAGTGGGTCCTAGAGATtt |
| SEQ ID NO: 145 | CCAACAGTGTGGCACGGAAtt |
| SEQ ID NO: 146 | GGACTTGGCAGGCAATTATtt |
| SEQ ID NO: 147 | CAGCAGTGGCAACCAGAATtt |
| SEQ ID NO: 148 | AATGAGAAGCAGATCGTGAtt |
| SEQ ID NO: 149 | GGAAGTGCATCATCGACATtt |
| SEQ ID NO: 150 | CAGCCAGGCCACATGACTAtt |
| SEQ ID NO: 151 | CCGCAGATGGGTTTAATGAtt |
| SEQ ID NO: 152 | ACACGTGGGCTGTGCGCATtt |
| SEQ ID NO: 153 | GAGAAGAACCACCCGGACTtt |
| SEQ ID NO: 154 | AGAAGAACCACCCGGACTTtt |
| SEQ ID NO: 155 | GCTGCAACTGCGACGGCTAtt |
| SEQ ID NO: 156 | ACGACTGACTTGCGGCAGAtt |
| SEQ ID NO: 157 | CCACACTGGCCACGACCTAtt |
| SEQ ID NO: 158 | GTACAGACCTCGAAGCCAGtt |
| SEQ ID NO: 159 | CAGCGAAGCCAACAACTATtt |
| SEQ ID NO: 160 | CCTTGGACCCTGTGGAGCAtt |

[symbol]

molecules of the invention. Preferably the agent identified affects an intracellular enzyme that processes Foxp3 into the double-cleaved short form (e.g., PC1, PC7, and Furin).

Agents of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

### V. Methods of Treatment

Many autoimmune disorders are the result of inappropriate or unwanted activation of T effector cells resulting in the production of cytokines and autoantibodies which mediate the disease process. In addition, Teff cell function is associated with graft rejection. Accordingly, when a reduced effector T cell or antibody response is desired, the compositions and methods of the invention can be used to down-modulate the expression and/or activity a molecule preferentially associated with Teff cells, e.g., such that at least one Teff cell function is down-modulated relative to at least one Treg cell function. In another embodiment, such disorders can be ameliorated by up-modulating the expression and/or activity of a molecule preferentially associated with T regulatory cells, e.g., such that at least one Treg cell function is up-modulated relative to at least one Teff cell function. One way that this can be accomplished is by removing cells from a patient, stimulating Treg activity using the methods described herein (i.e., increasing the activity of proprotein convertase enzymes, or delivering cleaved Foxp3 to cells), delivering the cells back to the patient for therapy. IN cases where it is desirable to deliver the Foxp3 peptides to a cell, uptake can be facilitated via encapsulation of the peptide into a liposome for example. As such, peptides ideally possess a nuclear localization signal, following entry into the cell, the peptide should be transported to the nucleus for exertion of biological activity.

In contrast, there are conditions that would benefit from enhancing at least one activity of Teff cells and/or down-modulating at least one activity of Treg cells. For example, immune effector cells often fail to react effectively with cancer cells. Accordingly, when an enhanced effector T cell or antibody response is desired, the methods of the invention can be used to regulate the expression and/or activity a molecule preferentially associated with Teff cells, e.g., such that at least one T effector cell function is up-modulated relative to at least one Treg cell function. In another embodiment, such disorders can be ameliorated by down-modulating the expression and/or activity of a molecule preferentially associated with T regulatory cells, e.g., such that at least one T regulatory cell function is down-modulated relative to at least one T effector cell function.

In an alternative approach for therapeutic uses, it is desirable to directly increase the production of single or double cleaved Foxp3 in cells. This could be accomplished by increasing the expression or activity of the enzymes responsible for processing Foxp3 into shorter active Foxp3 fragments.

It is clear from the foregoing that the single and double cleaved forms of Foxp3 can be used as powerful therapeutic agents. Delivery of the peptide to a patient can be accomplished by any known means in the art. One skilled in the art appreciates that a pharmaceutical composition comprising a single or double cleaved short Foxp3 can be administered to a subject by various routes including, for example, orally or parenterally, such as intravenously (i.v.), intramuscularly, subcutaneously, intraorbitally, intranasally, intracapsularly, intraperitoneally (i.p.), intracisternally, intra-articularly or by passive or facilitated absorption through the skin using, for example, a skin patch or transdermal iontophoresis, respectively.

A pharmaceutical composition comprising single or double cleaved short Foxp3 also can be incorporated, if desired, into liposomes, microspheres, microbubbles, or other polymer matrices (Gregoriadis, Liposome Technology, Vols. I to III, 2nd ed., CRC Press, Boca Raton Fla. (1993)). Liposomes, for example, which consist of phospholipids or other lipids, are nontoxic, physiologically acceptable and metabolizable carriers that are relatively simple to make and administer. Suitable pharmaceutical carriers and other agents of the compositions of the instant invention are described in “Remington's Pharmaceutical Sciences” by E. W. Martin (Mack Pub. Co., Easton, Pa.) and “Remington: The Science And Practice Of Pharmacy” by Alfonso R. Gennaro (Lippincott Williams & Wilkins, 2005). Therefore, the single or double cleaved Foxp3 can be delivered in liposomes via i.v. infusion.

The total treatment dose can be administered to a subject as a single dose or can be administered using a fractionated treatment protocol, in which multiple doses are administered over a more prolonged period of time, for example, over the period of a day to allow administration of a daily dosage or over a longer period of time to administer a dose over a desired period of time. A single or double cleaved short Foxp3 encoding nucleic acid can also be delivered in a vector selected from the group consisting of adenoviral vectors, plasmids, adeno-associated viral vectors, retroviral vectors, hybrid adeno-associated virus vectors, lentivirus vectors, pseudo-typed lentivirus vectors, herpes simplex virus vectors, and vaccinia virus vectors.

### VI. Kits

Kits are provided for practicing the methods of the instant invention. The kits comprise materials and reagents to facilitate the detection of the C-terminal Foxpeptide and native Foxp3 of the invention and instructional materials. Alternatively, the kit may comprise reagents suitable to modulate the activity of the enzymes responsible for cleaving the N- or C-terminal Foxpeptide region of Foxp3 such as the siRNA molecules disclosed herein. The pharmaceutical compositions can be included in a container, pack, or dispenser together with instructions for administration.

The following materials and methods are provided to facilitate practice of the present invention:

Mice—C57BL/6 (H-2^{b}) mice, purchased from The Jackson Laboratory, are housed under specific pathogen-free barrier conditions at the Children's Hospital of Philadelphia. Specific-pathogen free, female C57BL/6 mice (6-8 wk) (The Jackson Laboratory), and specific-pathogen free, female RAG−/− (C57BL/6) mice (Taconic Laboratories) were also used.

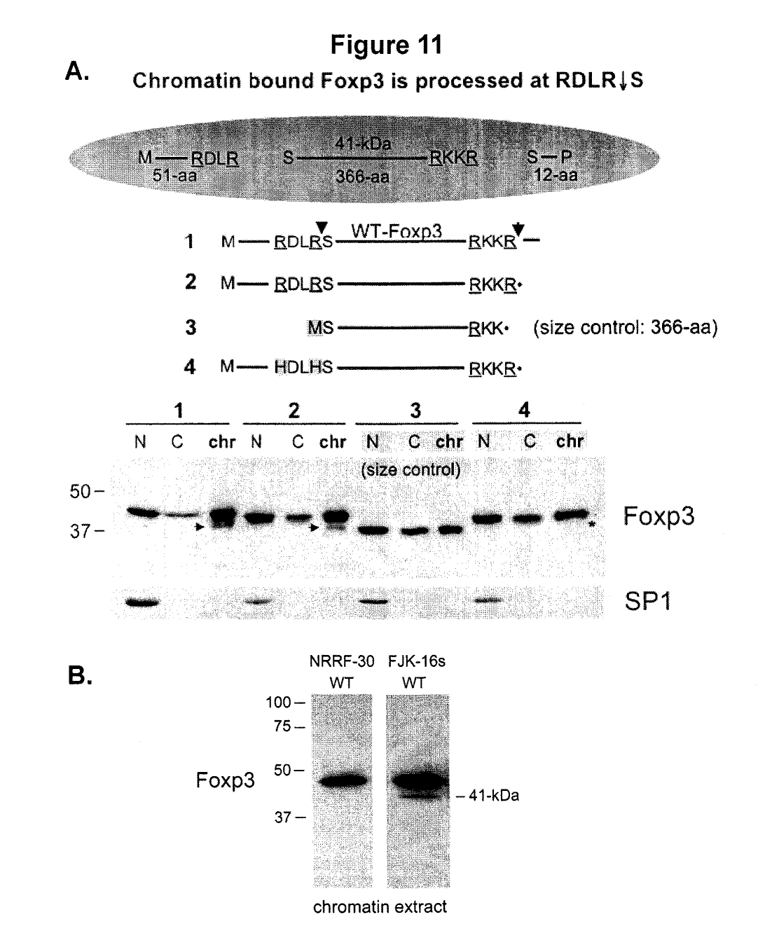

Antibodies—Polyclonal anti-murine Foxp3 Ab was generated by immunizing rabbits with a synthetic 11 aa peptide, QRPNKCSNPCP (SEQ ID NO: 186) (New England Peptide), corresponding to amino acids 419-429 of mouse Foxp3. This region is 75% conserved between species (3 amino acid differences between mouse and human). Foxp3 specific Ab was purified from high titer rabbit antiserum by affinity chromatography (Sigma-Genosys). Rat anti-mouse Foxp3 is purchased from ebioscience and used for flow cytometry and immunoperoxidase. “NRRF-30” mAb (ebioscience catalog #14-4771) which recognizes the N-terminus of full length Foxp3, and “FJK-16s” mAb (ebioscience catalog #14-5773) which recognizes the central domain of Foxp3 were used in FIG. 11B. “PCH101” mAb from ebioscience can also be used for recognition of the human Foxp3 N-terminal cleavage product.

Immunohistology—Cytospins of cells retrovirally expressing the different Foxp3 mutants were permeabilized with Triton X-100, followed by immunoperoxidase staining with rat anti-mouse Foxp3 mAb (eBioscience, cat #14-5773).

Cloning of mouse Foxp3 cDNA—ProStar Ultra HF RT-PCR system was used for cDNA generation from total thymus RNA and amplification of cDNAs; 1312-bp full-length Foxp3 cDNA was amplified by forward 5′-GAACCCAATGCCCAACCCTAG-3′ (SEQ ID NO: 175) and reverse 5′-TTCTTGGTTTTGAGGTCAAGGG (SEQ ID NO: 176) primers and cloned into pPCR-Script Bluescript vector. EcoR1 restriction sites were introduced by PCR amplification using Hotstart Pfu Ultra Hotstart Turbo DNA Polymerase (Stratagene) with forward 5′-GTGACCCGAATTCATGCCC AACCCTAGGCCAGCCAAG-3′ (SEQ ID NO: 177) and reverse 5′GAGGTTGGAATTCTCACCTCTTCTTGCA AACTCAAATTC-3′ (SEQ ID NO: 178) primers followed by cloning into Bluescript vector to yield pÖ737. Foxp3 cDNA fragment was purified after restriction digestion with EcoR1 and cloned into MINR-1 vector. Mutations were introduced during amplification via the reverse primer or with the Quick-change site II directed mutagenesis kit (Stratagene, cat #200523) using forward and reverse complementary primers (Integrated DNA Technologies). Following cloning into a Bluescript vector and sequence verification, all fragments were recloned into the Minr-1 vector for retroviral expression (69). Mutant constructs were also generated with appropriate primers using pÖ737 plasmid as the initial template.

In vitro mutagenesis of Foxp3—Foxp3 mutations were introduced with the Quick-change site II directed mutagenesis kit (Stratagene, cat #200523) using forward and reverse complementary primers purchased from Integrated DNA Technologies. Amino acid changes in the C-terminal end of Foxp3 were done by amplification with appropriate mutagenesis primers, followed by cloning into the SrfI cut pPCR-Script (Stratagene) vector for sequence verification.

Foxp3 retroviral construction—Retroviral vector Minr-1 was obtained from Dr. Andrew Wells. The Foxp3 mutant cDNAs were constructed by introducing the mutations by primers (Integrated DNA Technologies, Inc.) followed by synthesis and amplification with Hotstart Pfu Turbo Polymerase. The amplified Foxp3 constructs were then cloned into a Bluescript vector that had been linearized with the Srf I enzyme. These constructs were designed to have EcoR I restriction sites at both the immediate 5′ end (before ATG) and immediate 3′ end (after the TGA stop codon) of the genes. DNA sequences were validated at the CHOP Core DNA sequencing facility and Foxp3 specific forward and reverse primers were used in this process. The Foxp3 constructs were then cut with EcoR I, cloned into the Minr-1 vector previously linearized with EcoRI, followed again by sequence verification.

Foxp3 retroviral transduction—Minr-1 is a MSCV-based bicistronic vector, originally developed by Dr. Warren Pear which consists of a 5′ long terminal repeat (5′LTR) and 3′LTR sites and features an internal ribosome entry site (IRES) downstream of the cloning region and upstream of the non-signaling human nerve growth factor receptor (NGFR) gene used as marker.

Retrovirus was generated by co-transfection of wt-Foxp3 and Foxp3-mutants (in Minr-1) with pCLeco (Invitrogen) helper plasmid into the 293T-based Phoenix ecotropic packaging cell line. Briefly, Phoenix cells were plated at a density of 10×10⁶ cells/100 mm dish in 10 ml of RPMI medium (Invitrogen) containing 10% fetal bovine serum, 1× pen/strep, and 1× L-glutamine 24 hrs prior to transfection. Primary CD4+ T-cells isolated from spleen and lymph nodes by magnetic sorting were stimulated for 16-18 hrs in 24-well plates containing 1×10⁶ cells/well in 2 ml media with (3 ng/ml) PMA and (1 μM) ionomycin and 5 U/ml of IL-2.

At 48 h post-transfection of Phoenix cells, retroviral supernatants were harvested and utilized for transduction into CD4+ T-cells. The T-cells were transduced twice by spinfection with 48 hr and 72 hr viral supernatants obtained from transfected Phoenix cell media. Zero time was considered to be second of the two viral transductions performed one day apart. Transduced cells were expanded for 1-3 days unless otherwise stated, and used in suppression, ELISA assays, or for nuclear and cytoplasmic protein extraction.

Transduction efficiencies were determined by FACS analysis of human nerve growth factor receptor (hNGFR), expressed from the same bicistronic mRNA as Foxp3, or by direct measurement of Foxp3 expression on permeabilized cells, to show that the different mutants of Foxp3 were expressed at comparable levels. Typically the variation in transduction efficiencies between samples was <10%.

Testing retroviral Foxp3 expression—The Minr-1 retroviral constructs contain the reporter NGFR gene. NGFR expression levels have been shown to directly correlate with the level of expression of the upstream gene of interest (69). Assaying for NGFR expression in infected NIH3T3 cells as well as primary T-cells gives an accurate picture of the Foxp3 expression levels. NGFR expression was determined by FACS using an anti-NGFR mAb conjugated to PE or biotin. Expression of Foxp3 in transfected cells is also quantified by qPCR. Cells transfected with the null virus serve as negative controls in qPCR and in FACS analyses of Foxp3 expression. We have achieved successful transduction efficiencies of CD4+CD25− T-cells utilizing the Minr-l-wt-Foxp3 and Minr-1-mutant Foxp3 constructs and have demonstrated Foxp3 expression at the RNA and protein level.

Suppression assays—CD4+CD25+ (T-reg) and CD4+CD25− (T-eff) T cell and APC are isolated with magnetic beads following the manufacturer's instructions (Miltenyi). In some cases, 5×10⁴ CD4+CD25− T cells were labeled with CFSE and stimulated with CD3 mAb (1 μg/ml) in the presence of irradiated syngeneic APC and varying ratios of activated CD4+ cells that had been transduced with different Foxp3 constructs. Retrovirally transduced T-cells are serially diluted and co-cultured in different ratios (2:1, 1:1, 0.5:1 and 0.25:1) with CD4+CD25− T cells (1×10⁶/ml) and γ-irradiated (1000 rad) APCs (1×10⁶/ml) in TCM (RPMI 1640 complemented with 10% FBS, 100 μg and 100 unit of penicillin and streptomycin, respectively and 50 μM of β-mercaptoethanol), containing 0.5 μg/ml of anti-CD3. After 72 hours culture, cells were harvested and CD4+CD25− T cell proliferation was quantified by flow cytometry (Cyan) based on carboxylfluorescein diacetate succininyl ester (CFSE) profile of dividing effector T cells at 72 hr. Absolute numbers of effector T cells were determined by adding equal numbers of Dynabeads to each sample, and during FACS analysis gates were drawn on Dynabeads and collection time was based on set number of bead events for each sample. This allowed the collection of equal volume of sample from each tube and absolute numbers of cells were then based on gates designated after Flo-Jo software analysis.

RNA preparation—Total RNA is prepared from tissues using acid guanidine thiocyanate-phenol-chloroform followed by purification of RNA to remove contaminating DNA. Total RNA from cells is prepared by lysing the cells using the Qiashredder kit (Qiagen catalog #79654) followed by purification of RNA using the Qiagen RNAeasy Mini Kit (Qiagen cat #74104). All the RNA used in Q-PCR is DNAse treated during purification.

Quantitative PCR (qPCR)—Total RNA was prepared from 1-10×10⁶ cells by lysing cells with Qiashredder (Qiagen catalog #79654) followed by purification of RNA using the RNeasy Mini Kit (Qiagen, cat #74104), or alternatively, the RNA to be used for Q-PCR analysis was purified using an RNeasy Mini Kit (Qiagen), and samples were treated on RNeasy columns with DNAse Ito remove any contaminating DNA. To prevent PCR amplification of genomic DNA, sense and antisense primers were designed to be complimentary to different exons. qPCR-reactions were performed with an ABI Prism 7000 Analyzer using a 6-carboxy-fluorescein (FAM, reporter dye) at the 5′ end and with 6′carboxy-tetramethylrhodamine (TAMARA, quencher dye) at 3′ end. The probes were obtained from Applied Biosystems and primers from Integrated DNA Technologies. Foxp3 probe/primer is available from Applied Biosystems (cat #Mm00475156). cDNA was synthesized from 200 ng RNA with Multiscribe reverse transcriptase using random hexanucleotides, in a total volume of 100 μl. cDNA was synthesized with TaqMan reverse transcription reagents (Applied Biosystems), primer/probe sets were obtained from Applied Biosystems and qPCR performed using an ABI Prism 7000 Analyzer. qPCR reactions are done in 50 μl reaction volume with 2.5 μl template cDNA, 25 μl 2× Universal Master Mixture, 300 nM of each of forward and reverse primers and 250 nM of probe. The amplification profile includes an initial incubation at 50° C. for 2 min, denaturation at 95° C. for 10 min, and 40 cycles of 95° C. for 15 s and 60° C. for 1 min. A relative standard curve was used to quantify mRNA levels and the copy number of mRNA of interest by using the standard curve generated with the copy number of 18S ribosomal RNA. Control samples were also included in which reverse transcriptase was not added during the cDNA synthesis, and these were used as negative controls since signals generated from such samples would indicate the level of genomic DNA contamination. No measurable signals from the negative controls were normally obtained since DNAse I treatment, coupled with column purification of RNA results in complete removal of any contaminating DNA.

Western blots—Cells were lysed in SDS containing proteinase inhibitors; 25-75 μg protein was loaded onto large 14% or 20% SDS-PAGE gels. Nuclear and cytoplasmic extracts were prepared using a commercial kit NE-PER (Pierce, cat #78833). For analysis of nuclear and cytoplasmic proteins, 25 μg nuclear protein extract was loaded onto gels while keeping the cytoplasmic fraction at a constant ratio so as to reflect the correct distribution of nuclear and cytoplasmic proteins. Proteins were blotted onto PVDF membranes (Perkin Elmer, cat #NEF 1000) and probed overnight with the appropriate Abs, rinsed and probed with a secondary Ab-HRP conjugate followed by reaction with the Luminol reagent (Santa Cruz, cat #sc-2048) and exposure to Kodak Biomax MR film.

DNA and protein estimation—DNA was estimated by the diphenylamine-based color reaction assay originally described by Zacharias Dische (1930) and modified by Burton (76) in 1956. Protein-estimation was done using the Bio-Rad DC Protein Assay Kit (cat #5000112).

ELISA—Murine IFN-γ was assayed using a commercial kit (eBioscience, cat #88-7314).

Nuclear and cytoplasmic fractionation—Nuclear and cytoplasmic extracts were prepared using the commercial kit Ne-Per (Pierce, cat #78833) or as described in (83, 84). For analysis of nuclear and cytoplasmic proteins, 25 μg nuclear protein extract was loaded onto gels while keeping the cytoplasmic fraction at a constant ratio so as to reflect the correct distribution of nuclear and cytoplasmic proteins. Proteins were blotted onto PVDF membranes (Perkin Elmer, cat #NEF 1000) and probed overnight with the appropriate Abs, rinsed and probed with a secondary Ab-HRP conjugate followed by reaction with the Luminol reagent (Santa Cruz, cat #sc-2048) and exposure to Kodak Biomax MR film.

To show whether the proteolytic cleavage at the C-terminal end of Foxp3 takes place prior to binding of Foxp3 to DNA, a Foxp3 construct encoding a Flag-tag at the C-terminal end of Foxp3 was made and the Foxp3-Flag protein was retrovirally expressed in T-cells. The Foxp3:Flag ratio was then determined in total cell extract and compared to the Foxp3:Flag ratio obtained from DNA-bound Foxp3. Foxp3 was isolated as follows: Total Foxp3 was extracted from transduced T-cells expressing the C-terminal Flag-tagged Foxp3 by lysis through boiling in Laemmli sample buffer (w/5% β-Mercaptoethanol). DNA-bound Foxp3 was extracted from nuclei first by lysing the cells in 10 mM Hepes, pH 7.9, 10 mM KCl, 0.1 mM EDTA, 1 mM DTT, 0.5 mM PMSF, and 0.6% Nonidet-P40, followed by pelleting the nuclei through centrifugation. Next, the nuclei was extracted with 20 mM Hepes, pH 7.9, 400 mM NaCl, 1 mM EDTA, 1 mM DTT, 1 mM PMSF by incubation on ice for 1 hr with frequent vortexing. Following centrifugation, the insoluble material which represents DNA and bound material was pelleted and was extracted by boiling in the Laemmli sample buffer containing 5% β-Mercaptoethanol. Samples corresponding to Total Foxp3 and DNA-bound Foxp3 were analyzed by Western blotting on 14% SDS-Acrylamide gels. The two samples were loaded at different amounts and the lanes that gave equal Foxp3 signals in both samples were analyzed for the Flag signal, this was accomplished using duplicate membranes; one for Foxp3 detection and a different membrane for Flag detection.

Alternatively, 10×10⁶ T cells were incubated on ice in hypotonic buffer (10 mM HEPES, pH 7.9, 10 mM KCl, 0.1 mM EDTA, 1 mM DTT, 0.5 mM PMSF) for 15 min and lysed in the presence of 0.6% Nonidet P-40 by continued incubation on ice for another 15 min and frequent vortexing. Nuclei collected by brief centrifugation (30 sec, 14,000×g) were incubated in extraction buffer (20 mM HEPES, pH 7.9, 375 mM NaCl, 1 mM EDTA, 1 mM DTT and 1 mM PMSF) for 30 min incubation on ice with frequent vortexing. The nuclear extract and the DNA-bound material were separated by centrifugation at 4° C. for 15 min. DNA-bound material was dissolved by boiling in 1% SDS for DNA- and protein-analysis or directly boiled in Laemmli buffer for subsequent SDS-PAGE.

Adoptive transfer model of IBD—CD4+CD25− T cells were isolated from mesenteric lymph nodes using magnetic beads (Miltenyi) to >95% purity (by flow cytometry). CD4+CD25− T cells (1×106 cells) were co-injected i.p. into RAG1−/− (C57BL/6) mice along with Thy1.1+CD4+CD25− cells transduced with WT or mutated Foxp3 (1×105). Mice were monitored biweekly for clinical evidence of disease, including weight loss and stool consistency. RAG1−/− mice on the C57BL/6 background were purchased from Taconic Laboratory. Mice were housed in specific pathogen-free conditions, and used for studies approved by the institutional animal care and use committee of the Children's Hospital of Philadelphia.

Pathology—Gut samples were paraffin embedded and stained by H&E, or snap-frozen and stained by immunoperoxidase using mAbs to Foxp3 and T cell subsets (73).

Statistics—In vitro and in vivo suppression data were evaluated by ANOVA, and p<0.05.

The following examples are provided to illustrate embodiments of the invention. They are not intended to limit the scope of the invention in any way.

### Example I

Foxp3 Gets Cleaved at the RKKR↓S (SEQ ID NO: 188) Site to Yield a Shorter Protein Product that is Functional

The removal of the C-terminal 12 amino acids immediately following the RKKR (SEQ ID NO: 179) renders Foxp3 functional, indicating a block of function mediated by the neighboring C-terminal domain. Since these basic residues are involved in DNA binding (29), certain structural changes in the C-terminal domain should be taking place allowing interaction with DNA. This can be accomplished either through complex formation in which factors that complex with Foxp3 displace the C-terminal domain and expose the RKKR (SEQ ID NO: 179), or allowing interaction with DNA or simply by cleavage of Foxp3 immediately following the RKKR (SEQ ID NO: 179) sequence.

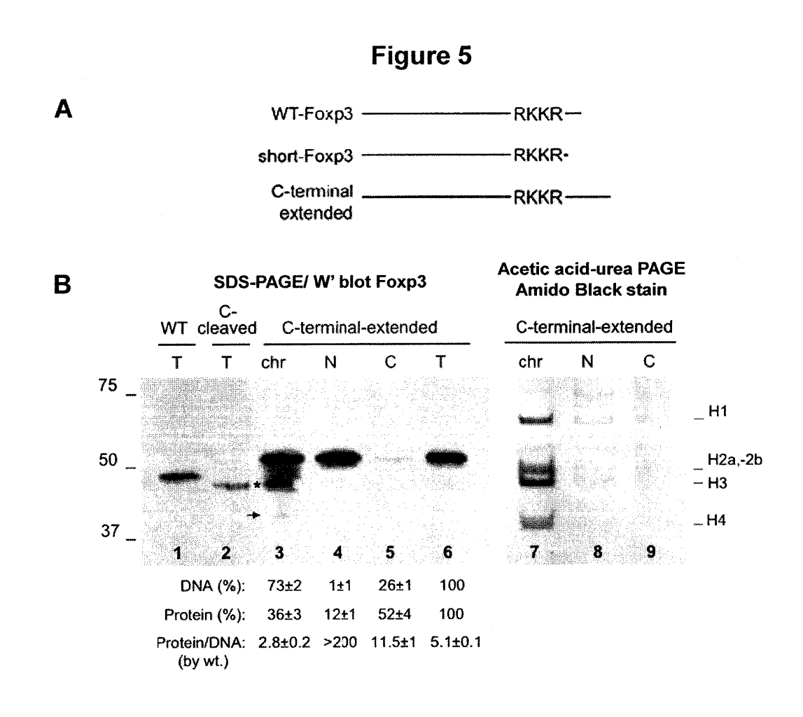

The first evidence that Foxp3 gets cleaved at the C-terminus was obtained using a C-terminal Flag-tagged Foxp3 construct. The flag-tagged Foxp3 was retrovirally expressed in cells and then the protein was detected by Western blot. Total cell extracts stained strongly for both the Foxp3 and Flag antibodies, while the insoluble nuclear fraction, which represents the DNA-bound material, stained only with the Foxp3 antibody, and very weakly with the Flag antibody (FIG. 1), indicating proteolytic cleavage of the C-terminal domain. Additional evidence was obtained by using a C-terminal-extended Foxp3 to resolve the uncleaved and C-terminal cleaved forms of Foxp3, discussed in Example II (FIG. 5A,B). The sequences of Foxp3 and some of its variant forms that result from processing are found in FIG. 2.

### Pro-Protein Convertases (PCs) and Foxp3 Processing

PCs are calcium dependent serine endoproteases responsible for activating a large number of substrates through cleavage. There are presently eight known members of this family. These include Furin, PC1 (also known as PC3), PC2, PACE4, PC4, PC5, PC7, SKI-1/SIP and PCSK9. Furin was identified in 1990 based on its structural similarity to the yeast Kex2 enzyme. The Furin consensus cleavage site R₄-X₃-(K/R)₂-R₁↓ (SEQ ID NO: 179) (SEQ ID NO: 180) was determined biochemically studying Furin substrates (33, 34). The sequence R₄X₃X₂R₁↓ (SEQ ID NO: 180) represents the minimal cleavage site, however, favorable residues at position 2 and position 6 (2 aa preceding R₄;) were found to compensate for less favorable ones at position 1 (35).

In vitro studies indicate that the substrate specificity of different PCs overlap, but gene knock-out studies indicate that there is only partial functional redundance. Furin, PC1, and PACE4 null mice are embryonically lethal and display distinct lethality phenotypes (36-38), on the other hand, PC2 null mice are diabetic (39) and male PC4 null mice are infertile (40).

PC1, unlike Furin which is broadly expressed, is selectively expressed in neuroendocrine tissues including brain (41-43). PC1 shows a similar recognition pattern to Furin, however, it has a strong preference for K to be present at position three (44). The R—X—K—R (SEQ ID NO: 187) sequence is also a known sequence for cleavage by PC1. Cleavage preference for other members varies, however, a consensus sequence that includes all the PCs can be shown as (R/K)—(X)ₙ—(R/K)↓, where the vertical arrow represents cleavage; X is any amino acid other than cysteine, n is number of residues (n=0, 2, 4, 6 residues) (45).

Following the discovery of OP-1 and OP-2 (BMP-7 and BMP-8), the alignment of the pro-protein sequences of several TGF-β superfamily members resulted in the identification of the RXXR (SEQ ID NO: 180) sequence as the cleavage site necessary for the maturation of this protein family (46). The proposed mechanism was validated experimentally by several groups demonstrating pro-protein convertase (PC) family members cleave TGF-β family members past RXXR (SEQ ID NO: 180) sequences (47, 48).

Furin is a type I transmembrane protein mainly localized to trans-Golgi network (TGN), however, it cycles between TGN, cell surface and early endosomes (49). Furin is also secreted possibly as a result of post-translational modification (50). The list of proteins processed by Furin is extensive; at the TGN it cleaves pro-BMP-4 (47), pro-β-NGF (51) and IGF1 and IGF1R-R (52), and at the cell surface it cleaves and activates anthrax protective antigen (PA), and ectodysplasin-I (Eda-I) (53). In the mildly acidic environment of early endosomes it can cleave the diphtheria toxin and the shiga toxin. Furin was shown to cleave the CXCR3 specific chemokine CXCL10 (IP-10) close to its C-terminal end, releasing the C-terminal 4 amino acids following cleavage (54). Recently, Furin expression was found to be upregulated in T helper 1 cells following IL-12 treatment, resulting in enhanced IFN-γ secretion, conversely, inhibition of Furin resulted in the loss of IFN-γ production (55).

The RKKR (SEQ ID NO: 179) proteolytic maturation motif found in Foxp3 is identical to that of BMP-3, TGF-β2, -β3, -β5, and very similar to other members of the TGF-β superfamily. Therefore, Foxp3 appears to be cleaved by the proprotein convertase family of enzymes, for example, PC1, with cleavage releasing the C-terminal 12-amino acid peptide (i.e., Foxpeptide).

The previous examples indicate that only the full-length Foxp3 depends on an intact 414RXXR417 (SEQ ID NO: 180) pattern for proper nuclear localization. A likely scenario that would result in the exposure of the RKKR (SEQ ID NO: 179) sequence would be through cleavage and subsequent release of the C-terminal domain by specific protease(s) mentioned above. The fact that RKKRS (SEQ ID NO: 188) is a recognition sequence for Furin, an endoprotease that is a member of the pro-protein convertase (PC) family, makes proteolytic cleavage a likely mechanism that regulates the function of the C-terminal domain. The PC family of enzymes cut immediately following an RXXR (SEQ ID NO: 180) pattern and the sequence RKKRS (SEQ ID NO: 188), conserved in both the human and mouse Foxp3s, is reportedly an ideal Furin recognition sequence (59). Cleavage at this site would result in the release of the 12-amino acid C-terminal domain from the end of Foxp3. Another enzyme that may recognize the cleavage site in Foxp3 is PC7 which shares many characteristics with Furin and PC1.

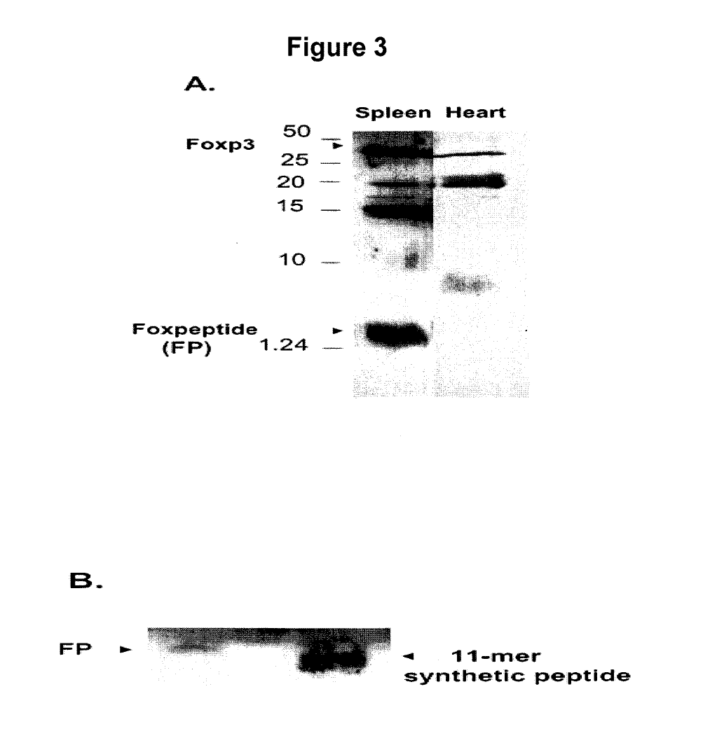

To determine whether the RXXRS (SEQ ID NO: 188) sequence gets cleaved, an antibody was raised in rabbits against a synthetic peptide consisting of the last 11 amino acids of Foxp3. This anti-peptide Ab was then used in Western blots of spleen and heart extracts to check for the presence of a cleaved C-terminal Foxp3 peptide. After separation on large, 20% SDS-PAGE gels, a fragment of approximately 1.5 kDa, consistent with the molecular weight of a 12-amino acid peptide (theoretical mol. wt. 1.33 kDa) was detected in spleen but not in heart, a non-lymphoid organ (FIG. 3A).

The synthetic peptide used in raising the polyclonal Ab was run side by side with a spleen extract, and as seen (FIG. 3B) the 11-mer synthetic peptide migrates slightly faster than the 12-amino acid peptide. This peptide, cleaved from the C-terminal end of Foxp3, is termed Foxpeptide. The present data support a scenario in which cleavage and release of the Foxpeptide would result in the exposure of the RKKR (SEQ ID NO: 179), allowing interaction with factors that form complexes with Foxp3.

RXXR (SEQ ID NO: 180) motif represents a potential recognition sequence for cleavage by the enzymes of the PC family, and our identification of two such motifs in murine and human Foxp3 suggested to us that Foxp3 might be processed by this mechanism. Foxp3 is unique in that none of the other Foxp family members have an RXXR (SEQ ID NO: 180) motif; i.e. a glutamine residue (Q) is found in place of the first arginine (QKRR) (SEQ IDNO: 189). Moreover, in the cases of Foxp1 and Foxp4, QKRR (SEQ ID NO: 189) sequences are followed by a proline residue (QKRRP) (SEQ ID NO: 190) at position P1′. While serine is the most frequently reported amino acid at P1′, proline has not been reported in surveys of PC cleavage sites (34). One of the two RXXR (SEQ ID NO: 180) motifs in Foxp3, RDLR (SEQ ID NO: 183), is at the N-terminal side of Foxp3, and the other, RKKR (SEQ ID NO: 179), is very close to the C-terminal end. Proteolytic cleavage at RKKR (SEQ ID NO: 179) was demonstrated by detection of the released C-terminal peptide on Western blots using an antibody raised against the last 11-aa of Foxp3.

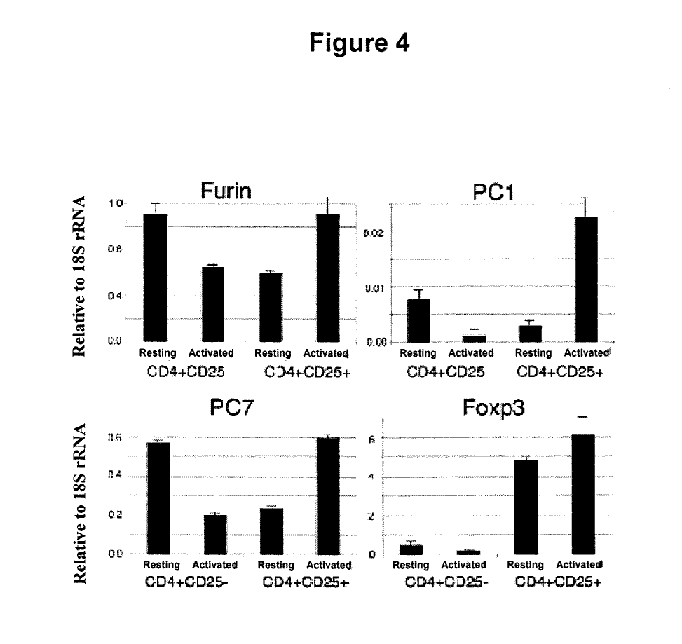

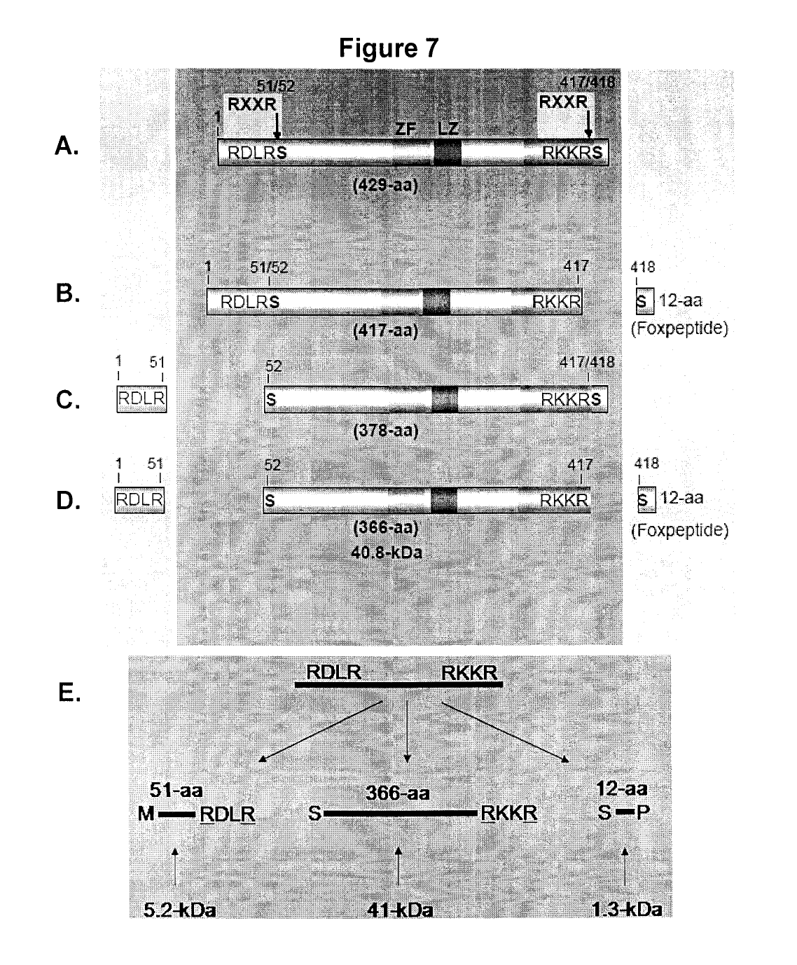

Quantitative PCR was used to determine expression of the seven main PC enzymes (PC1/3, PC2, PC4, PC5/6, PC7, PACE4, Furin) in resting versus activated CD4+CD25− and CD4+CD25+ cells. Levels of PC2, PC4, PC5 and PACE4 mRNA were too low to be detected, whereas Furin, PC1 and PC7 mRNA levels were readily detected. These three PC had contrasting expression patterns in CD4+CD25− vs. CD4+CD25+ cells, and in resting vs. activated cells (FIG. 4). While activation of CD4+CD25− cells resulted in a marked decrease in Furin, PC1 and PC7 mRNA levels, these genes showed increased expression in activated CD4+CD25+ cells (FIG. 7). The greatest increase, 8-10 fold, was found for PC1 mRNA, following activation of CD25+ cells with CD3 mAb or PMA plus ionomycin. These data indicate that several PC are expressed in CD4+CD25+ T cells and their higher level of expression upon activation is consistent with a mechanism in which Foxp3 matures through proteolytic cleavage. Of these PC, PC1 has been shown to have a functional nuclear localizing sequence (56) and may be responsible for processing chromatin-associated Foxp3 to its active state.

### Example II

### Proteolytic Processing of Foxp3 Occurs at the Chromatin Level

Several attempts were made to resolve the cleaved and uncleaved forms of Foxp3 by SDS-PAGE, but the small size difference (12-aa) between the two forms proved challenging. To assist with resolution by SDS-PAGE, the C-terminal tail (sequences past RKKR (SEQ ID NO: 179)) was extended from 12-aa to 31-aa (FIG. 5A). Cytoplasmic and nuclear extracts from CD4+ T cells transduced with retroviruses encoding wild-type or extended Foxp3 were analyzed by Western blotting for cleaved and uncleaved forms. These extracts revealed only a single Foxp3 species that corresponded to the uncleaved (C-terminal-extended) Foxp3 (FIG. 5B, lanes 4 and 5).

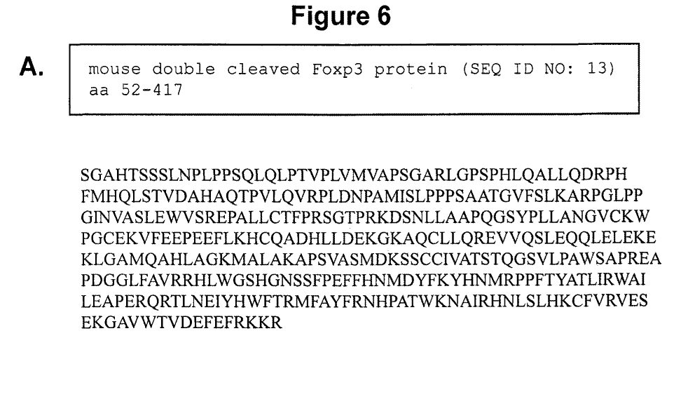

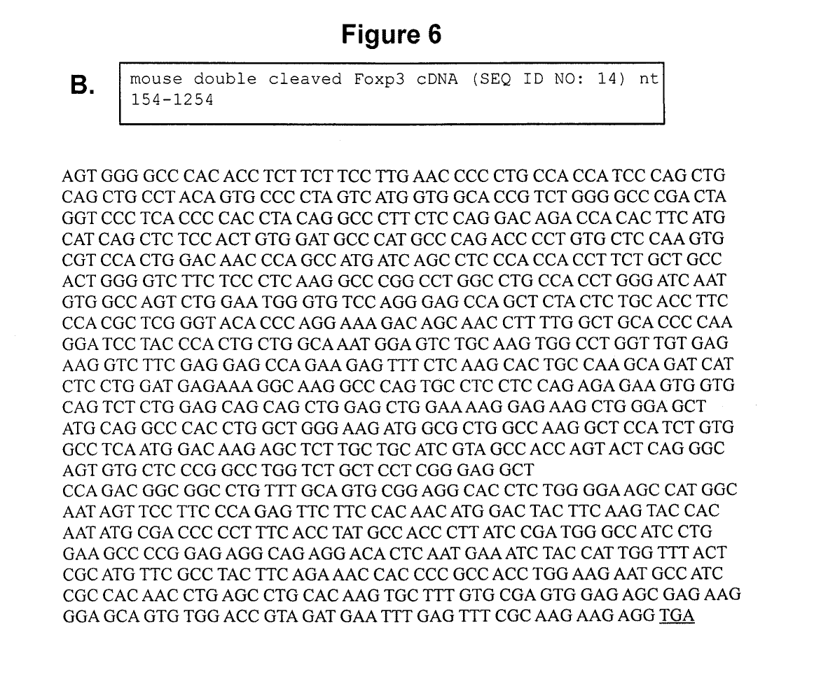

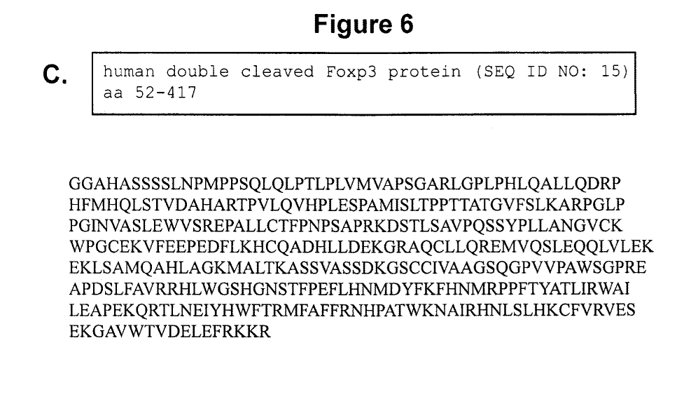

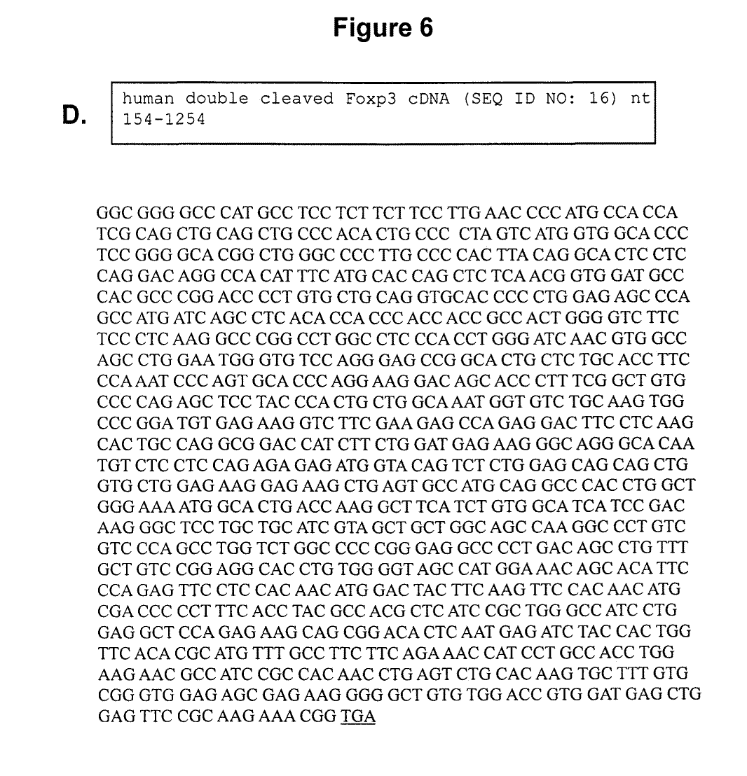

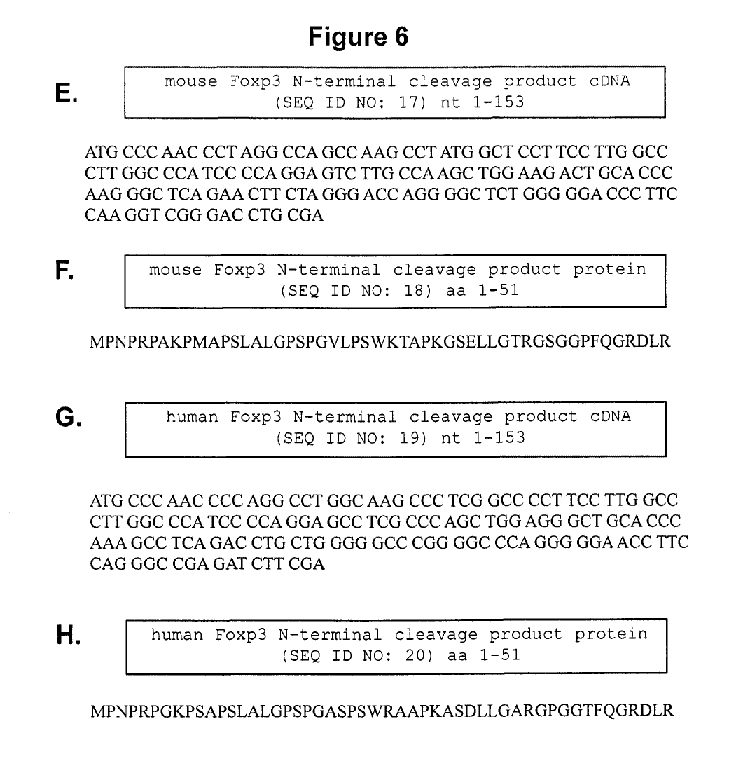

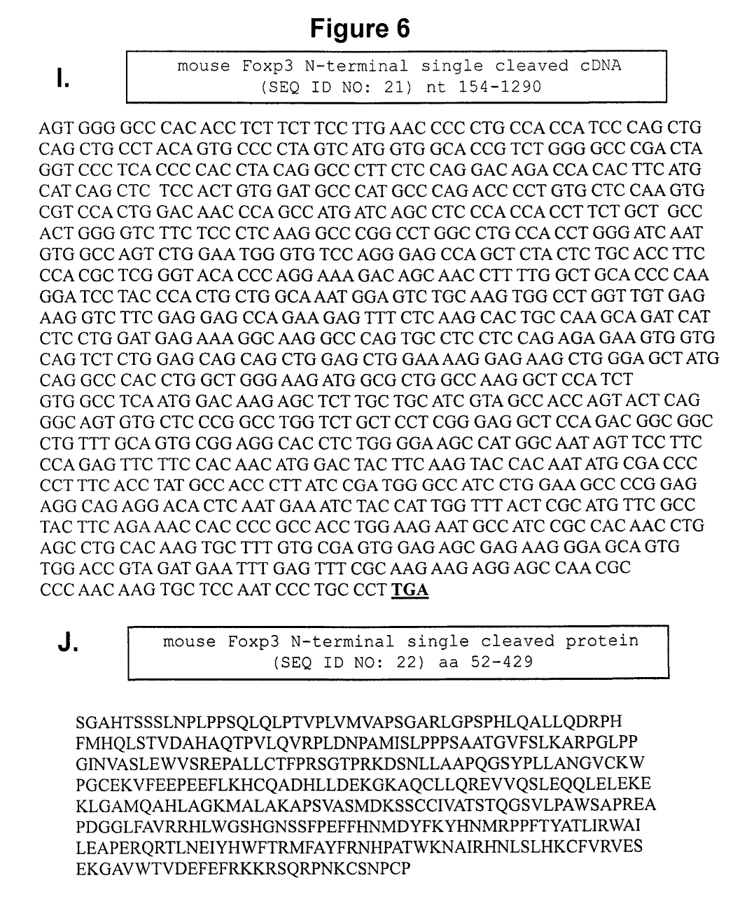

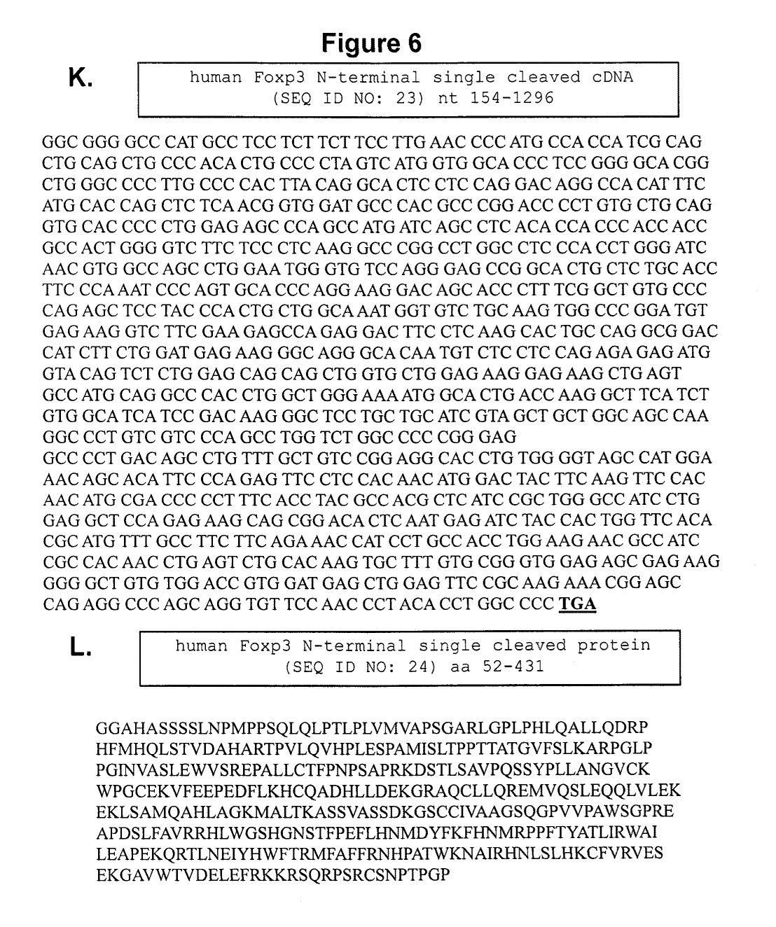

Using material from these experiments, the chromatin-associated material that remained after nuclear extraction was analyzed. The protein:DNA ratio (by weight) in this fraction was approximately 2.8:1, a characteristic ratio for chromatin (74). Two major species of Foxp3 were detected, a slow migrating species corresponding to the uncleaved (C-terminal-extended) Foxp3 and a faster migrating Foxp3 species. The fast migrating species was found to co-migrate with the engineered short form of Foxp3 that mimics a cleaved product (size control), indicating it is the proteolytically processed form of Foxp3 (FIG. 5B, lanes 2 and 3). A third species, approximately 40 kDa in mol. wt., was also faintly visible on the Western blots, and likely represents a form of Foxp3 cleaved at both the N-terminal and C-terminal RXXR (SEQ ID NO: 180) motifs. The putative sequence of the double cleaved forms of mouse and human Foxp3 are shown in FIG. 6A-D.

Expression of an engineered C-terminal extended Foxp3 in CD4+ T cells allowed the detection of the two forms, cleaved and uncleaved Foxp3, by SDS-PAGE. A third species, approximately 41-kDa in molecular weight, was also faintly detected in the chromatin-associated fraction (FIG. 5B). This is the putative double cleaved short Foxp3. Notably the same R—X—X—R (SEQ ID NO: 180) proprotein convertase recognition site is found within the first 51 amino acids of several Foxp family member proteins. More specifically, prior to the RXXR (SEQ ID NO: 180) motif, Foxp1 shares only 43% identity with the first 51 N-terminal amino acids of Foxp2, and Foxp2 shares only 16% identity with the first 51 N-terminal amino acids of Foxp3, however, despite such low homology between these members, the RXXR (SEQ ID NO: 180) motif is present in all three of them. The double cleaved (N- and C-terminal) Foxp3 sequences for the mouse and human forms are depicted in FIG. 6, panels A-D. Additionally, the sequences of the N-terminal cleavage product of mouse and human Foxp3 are shown in FIG. 6, panels E-H. FIG. 6, panels I-L show the sequence information of the Foxp3 forms that are singly cleaved at the N-terminus. The N-terminal cleavage product, shown in FIG. 6, panels E-H, could also be used for detecting activated regulatory T-cells in the same way that the Foxpeptide fragment can be utilized. Antibodies can be raised to this region to facilitate detection. FIG. 6, panels I-L show the sequences of mouse and human Foxp3 which has been cleaved only at the N-terminal RXXR (SEQ ID NO: 180) motif. FIG. 7, panels A-F is a schematic diagram which shows the different forms of Foxp3 resulting from single or double cleavage at the N- and C-terminal RXXR (SEQ ID NO: 180) motifs.

### Example III

### Generation of Short-Foxp3 Depends on an Intact RXXR (SEQ ID NO: 180) Motif

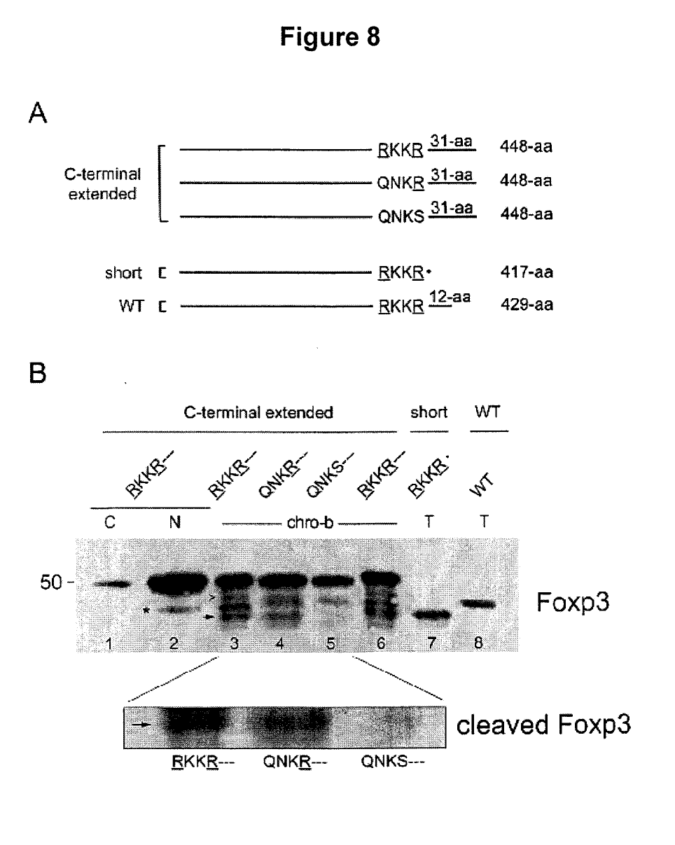

Since the C-terminal extensions allowed the cleaved and uncleaved forms of Foxp3 to be resolved by SDS-PAGE, the C-terminal extended construct was used as a template and further mutations were introduced into the RKKR (SEQ ID NO: 179) sequence to probe the role of the RXXR (SEQ ID NO: 180) motif (FIG. 8A). Constructs, each with 31-aa long tails (denoted with dashes, ---), were retrovirally expressed and cellular fractions analyzed by Western blotting. The short cleaved form of Foxp3 was again exclusively detected in the chromatin fraction (FIG. 8B, lane 3, marked with arrow) and could not be detected in the nuclear extract (FIG. 8B, lane 2), which contained uncleaved C-terminal extended and endogenous Foxp3 (FIG. 8B, lane 2, marked with asterisk). Expression of the mutant QNKR--- (SEQ ID NO: 181) showed the loss of the first arginine (R) results in much lower levels of the cleaved form (lane 4), and loss of both arginine residues (QNKS---) (SEQ ID NO: 182) leads to a complete lack of proteolytic cleavage (lane 5). These experiments demonstrate Foxp3 is cleaved at the C-terminal end and that RKKR (SEQ ID NO: 179) is required for recognition and enzymatic cleavage. A second engineered Foxp3 mutant with a longer C-terminal extension (40-aa) does not get cleaved, indicating the additional amino acids result in a topological change in the C-terminal domain and prevent enzymatic recognition.

That an intact RXXR (SEQ ID NO: 180) is required for cleavage was demonstrated by introducing mutations into the RKKR (SEQ ID NO: 179) sequence of the C-terminal-extended Foxp3. While the loss of the first arginine (QNKR) (SEQ ID NO: 181) resulted in partial processing, loss of both arginines (QNKS) (SEQ ID NO: 182) completely prevented proteolytic cleavage (FIG. 8). To determine whether the cleaved form of Foxp3 differs in activity from the uncleaved form several Foxp3 constructs were made that encode proteins mimicking either cleaved Foxp3 (417-aa) or a cleavage resistant Foxp3 (429-aa). Short-Foxp3 constructs were made by insertion of a stop codon immediately past the 416KR417 sequence (RKKR• (SEQ ID NO: 179) or QNKR• (SEQ ID NO: 181)). In the long-Foxp3 construct, the C-terminal RXXR (SEQ ID NO: 180) motif was abolished while the DNA-binding residues were kept intact (RKKR (SEQ ID NO: 179) was replaced with QNKR (SEQ ID NO: 181)). Constructs QNKR• (SEQ ID NO: 181) and QNKR-- (SEQ ID NO: 181) carry the same mutation but one encodes a short Foxp3 without the C12 terminal tail, and the other a long Foxp3 with a relatively cleavage resistant tail. These two constructs were used to determine the affect of proteolytic cleavage on the activity of Foxp3.

### Example IV

### Expression and Nuclear Localization of Foxp3 Mutants

To further study the role of the RKKR (SEQ ID NO: 179) sequence in Foxp3 function, four Foxp3 constructs were prepared by site-directed mutagenesis. The first encodes a short 417-aa Foxp3, ending with 414RKKR417 (SEQ ID NO: 179) (RKKR• (SEQ ID NO: 179) in FIG. 5), while the second construct encodes a short 417-aa Foxp3 in which the last four amino acids (RKKR) (SEQ ID NO: 179) are replaced with the unrelated amino acids PNNW (SEQ ID NO: 184) (referred to as PNNW• (SEQ ID NO: 184)). The last two mutants (referred to as QNKR• (SEQ ID NO: 181) and QNKR-- (SEQ ID NO: 181)) have identical mutations that abolish the C-terminal RXXR (SEQ ID NO: 180) proteolytic cleavage recognition motif (RKKR (SEQ ID NO: 179) replaced with QNKR (SEQ ID NO: 181)); in these two mutants the DNA-binding residues, KR, are kept intact. The short QNKR (SEQ ID NO: 181) mutant (QNKR•) (SEQ ID NO: 181) does not have a C-terminal tail (Foxpeptide domain) and terminates with QNKR (SEQ ID NO: 181) (length is 417-aa instead of 429-aa). The long QNKR (SEQ ID NO: 181) mutant (QNKR--) (SEQ ID NO: 181) has the same mutation as the short QNKR• (SEQ ID NO: 181), the difference being QNKR-- (SEQ ID NO: 181) has an intact C-terminal tail rendered resistant to proteolytic cleavage due to destruction of the RXXR (SEQ ID NO: 180) motif. Loss of the first arginine residue in the RXXR (SEQ ID NO: 180) motif, as in the QNKR-- (SEQ ID NO: 181) mutant, reportedly results in a much lower cleavage rate (59). The QNKR-- (SEQ ID NO: 181) and QNKR• (SEQ ID NO: 181) mutants were designed to reveal the role of proteolytic cleavage in Foxp3 function.

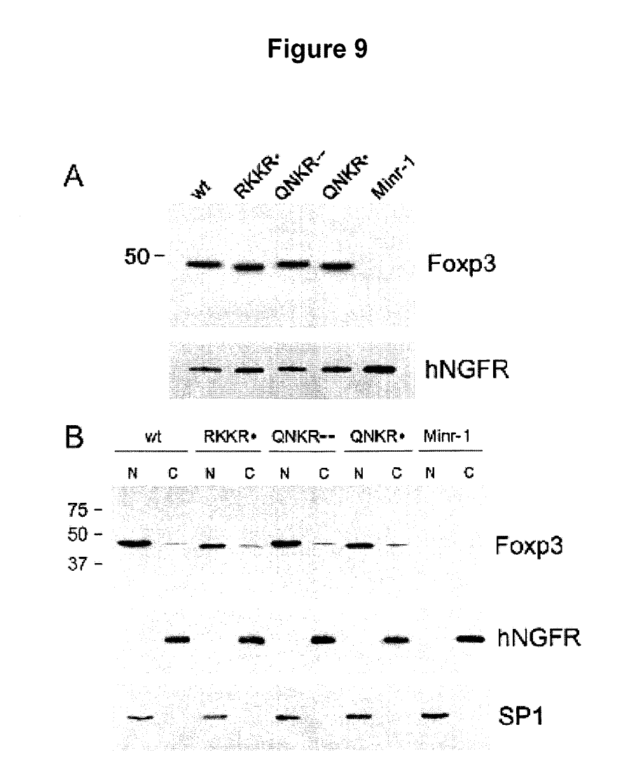

Foxp3 mutants were cloned from Bluescript into Minr-1 vector and expressed by the same bicistronic message as hNGFR in CD4+ T cells via retroviral transduction. Assessment of individual Foxp3 mutants and their relative levels of hNGFR at 4 d post-transduction showed the Foxp3 mutants were equally expressed and were equally stable (FIG. 9A). The nuclear transport properties of WT-Foxp3 and engineered Foxp3 mutants were studied in transduced CD4+ cells after separation of nuclear and cytoplasmic components at 4 d post-transduction (FIG. 9B). Foxp3 mutants had similar nuclear localization profiles and RKKR (SEQ ID NO: 179) mutations did not affect the nuclear translocation of Foxp3 mutants (FIG. 8B), indicating that RKKR (SEQ ID NO: 179) does not function as a nuclear localization sequence in Foxp3, and in contrast to data in which nuclear localization of a Foxp3-GFP fusion protein was studied in a non-T cell line (HEK293 cells) (63). The differences in our observations can be explained by their use of a GFP-Foxp3 hybrid in which the GFP epitopes (27 kDa) may prevent the Foxp3 end of the hybrid from properly folding, and by their use of a non-T cell line. Analysis on the same Western blot of hNGFR (cytoplasmic) and SP1 transcription factor (nuclear) expression showed nuclear and cytoplasmic fractions were separated cleanly, with minimal cross-contamination (FIG. 9B).

Since the basic RXXR (SEQ ID NO: 180) sequence may function as a cleavage target and NLS, it was the focus of further study. All constructs were first tested for equal expression and for their nuclear transport ability prior to suppression assays and animals studies. Mutations in the RKKR (SEQ ID NO: 179) sequence of the full-length Foxp3 were found not to affect the nuclear transport ability of the protein (FIG. 8B).

### Example V

### Foxp3 is Cleaved at Both the N-Terminal (RDLR) (SEQ ID NO: 183) and C-Terminal (RKKR) (SEQ ID NO: 179) RXXR (SEQ ID NO: 180) Sites

Foxp3 is activated by a mechanism that involves PCs, with proteolytic cleavage resulting in release of both N- and C-terminal ends (51-aa and 12-aa, respectively). The double cleaved short Foxp3 is present only in the chromatin fraction, indicating Foxp3 is processed following its interaction with DNA, and that proteolytic cleavage is required for its activity. Of the known PCs, PC1 expression is increased markedly in Tregs upon activation and is equipped with a functional NLS (56). The C-terminal RXXR (SEQ ID NO: 180) motif in Foxp3 (RKKR) (SEQ ID NO: 179) sequence, fulfills the recognition criteria for PC1. Thus, PC1 appears to be the enzyme responsible for Foxp3 activation.

The search for RXXR (SEQ ID NO: 180) motifs in the other FoxP family members revealed Foxp1, 2 and 3 all have an RXXR (SEQ ID NO: 180) motif close to their N-termini. Interestingly, Foxp1, -2 and -3 do not have any significant homology outside their forkhead domains. Foxp3 is only 18% identical to Foxp1 and 16% to Foxp2 within the first N-terminal 51-amino acids where the RXXR (SEQ ID NO: 180) motifs are located. The fact that all three members have an RXXR (SEQ ID NO: 180) motif despite a lack of significant homology suggests N-terminal recognition and processing by proprotein convertases may be a common mechanism by which members of this family are activated through removal of their N-terminal ends.

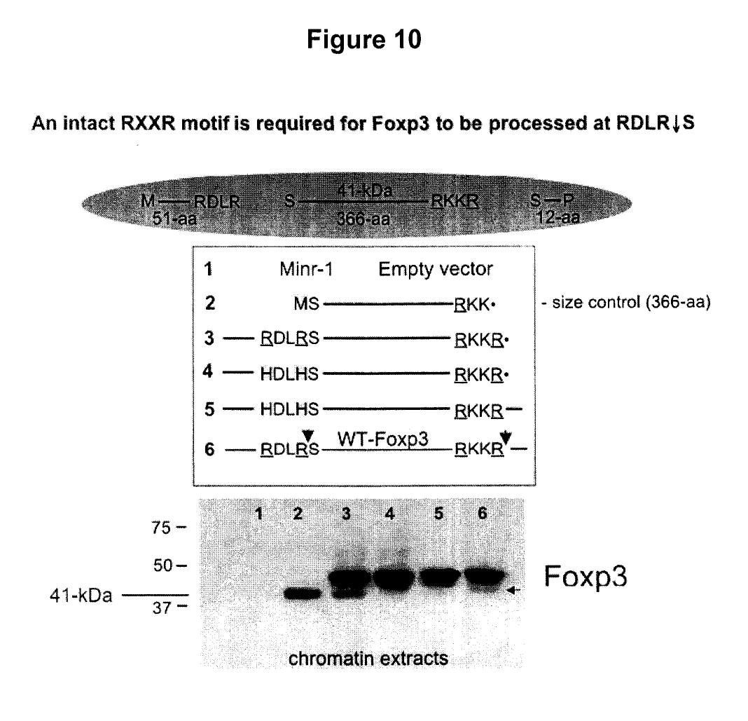

A 41-kDa Foxp3 species on Western blots was detected that represents a form of Foxp3 (366-aa) cleaved at both N-terminal and C-terminal RXXR (SEQ ID NO: 180) motifs (48RDLR↓S52 (SEQ ID NO: 191) and 414RKKR↓S418) (SEQ ID NO: 188). To assess whether the 41-kDa species detected on Western blots could be Foxp3 that has lost both its N-terminal and C-terminal ends through proteolytic cleavage, the migration property of the 41-kDa species was compared to that of an engineered Foxp3 (used as a size control). This control Foxp3 lacks the N-terminal 51-aa and the C-terminal tail sequences and has exactly the same number of residues as a double-cleaved Foxp3 (366-aa). Western blot analysis showed the engineered “size control” protein co-migrated with the 41-kDa Foxp3 species (FIG. 10, lanes 2,3 and 6), indicating cleavage of Foxp3 at both N-terminal and C-terminal RXXR (SEQ ID NO: 180) motifs is most likely responsible for the generation of the 41-kDa species. The dependence of N-terminal proteolytic cleavage on an intact RXXR (SEQ ID NO: 180) motif was then demonstrated by replacement of the two arginines with histidines, a basic amino acid. Two constructs were made, a full length construct (429-aa) (FIG. 10 lane 5) and another lacking the C-terminal tail (417-aa)(FIG. 10, lane 4; constructs with mutant amino acid residues yellow highlighted in Figure), each bearing the RDLR (SEQ ID NO: 183) to HDLH (SEQ ID NO: 192) mutation that destroy the RXXR (SEQ ID NO: 180) motif at residues 48 and 51. Analysis of chromatin-bound Foxp3 on Western blot (FIG. 10, comparison of lanes 3 and 6 with 4 and 5) showed the loss of the motif results in total disappearance of the 41-kDa Foxp3 species, proving that the 41-kDa species is the direct result of proteolytic cleavage of the N-terminal end.

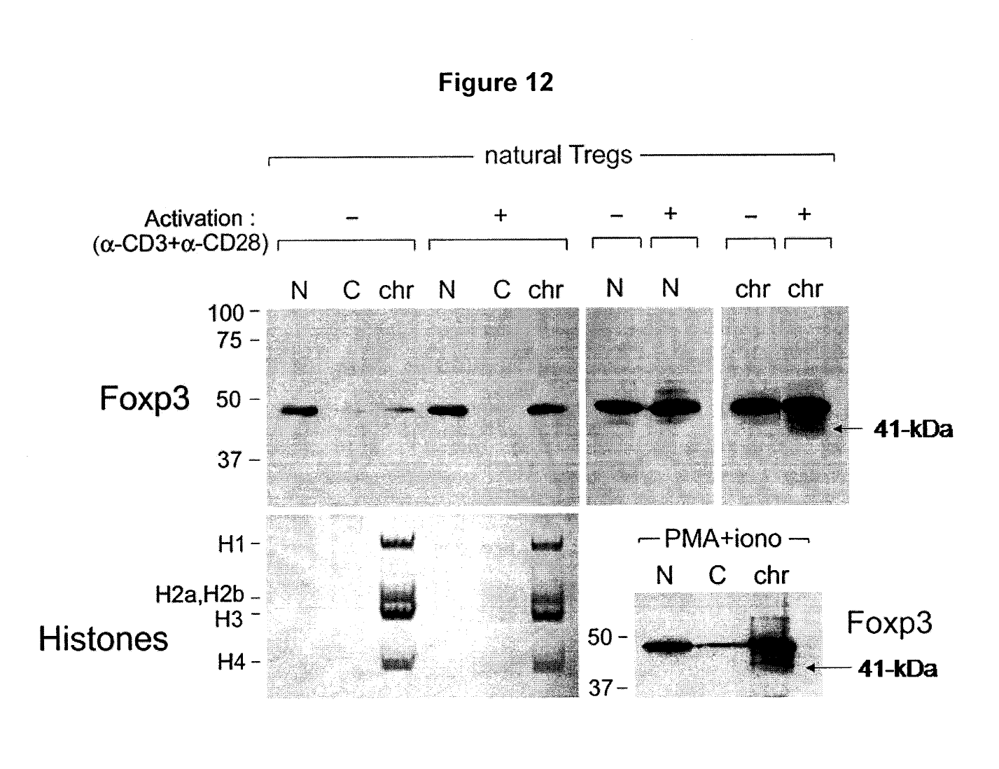

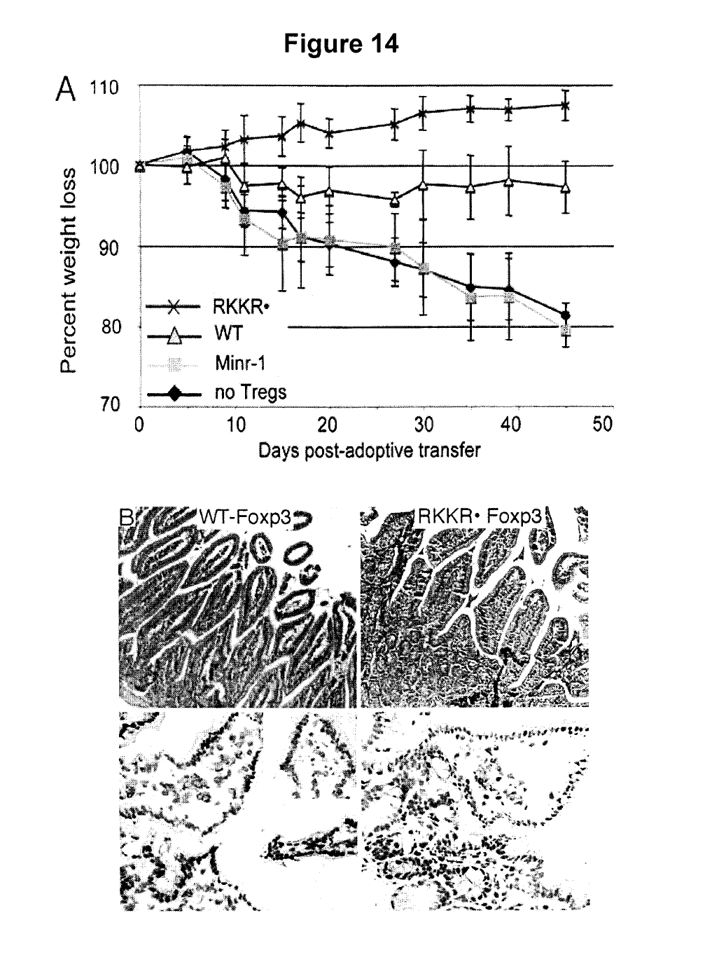

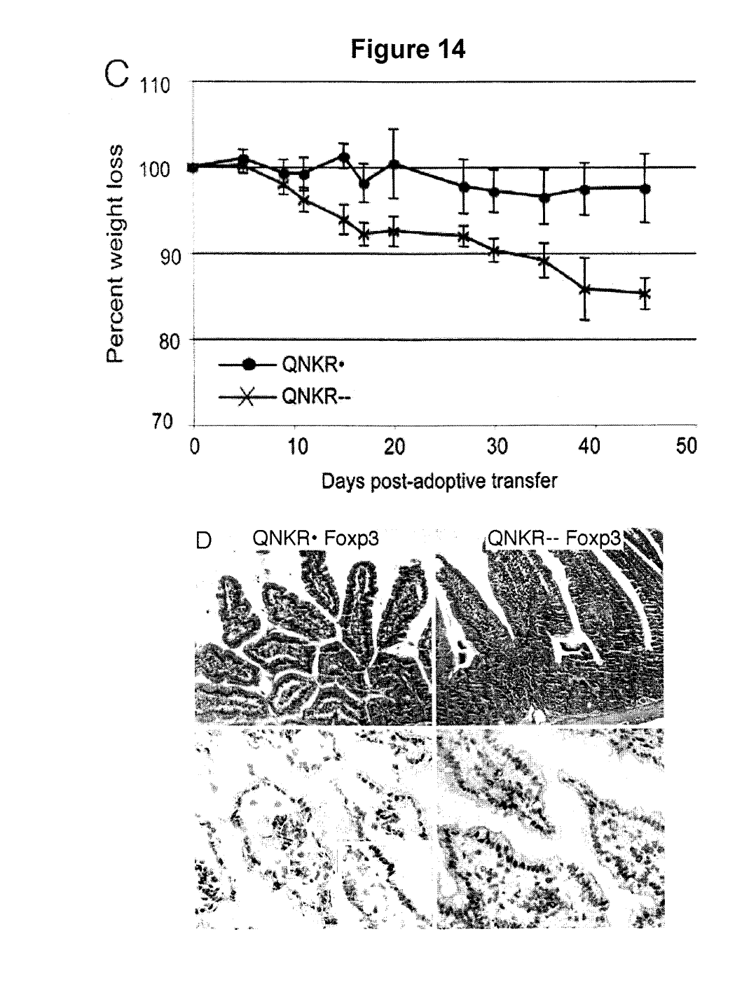

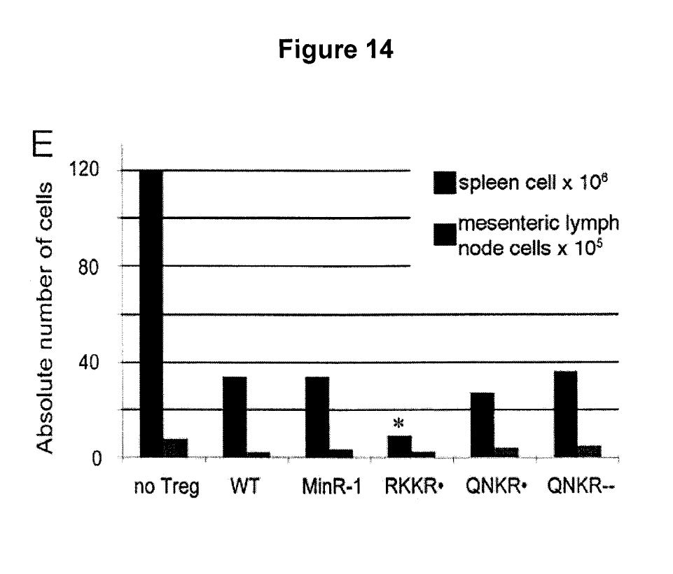

The subcellular distribution of the 41-kDa species was then studied. This shorter species was again not detected in the nuclear or cytoplasmic extracts but was confined to the chromatin fraction (FIG. 11A). The asterisk at the right side of FIG. 14 shows the absence of any 41-kD species in Foxp3 lacking the N-terminal RXXR (SEQ ID NO: 180) motif (FIG. 11A, sample 4). FIG. 11B is a Western blot of a chromatin extract from CD4+ cells retrovirally expressing Foxp3. The same extract was run on two lanes; the first lane was incubated with NRRF-30 mAb which recognizes the very N-terminal end of Foxp3, and the second lane was incubated with FJK-16s mAb which recognizes a site in the central region of Foxp3 beyond the N-terminal cleavage site. Importantly, NRRF-30 does not recognize the 41-kDa Foxp3 species, demonstrating that the 41-kDa species is the result of N-terminal cleavage. FIG. 12 is a western blot showing that only activated Tregs express the 41 kDa Foxp3 (double cleaved) species; this double-cleaved short Foxp3 is detectable only in activated natural Tregs in the chromatin-bound fraction (Foxp3 was activated by both antibodies and PMA). This data demonstrates that the double-cleaved short Foxp3 is the functional form of Foxp3.

### Example VI

C-Terminal-Cleaved Foxp3 Missing the Last 12-aa is Functional and Rransduced CD4+ Cells Expressing C-Cleaved-Foxp3 have Higher Suppressive Activity than WT-Foxp3

WT Foxp3 and engineered Foxp3 mutants (N-terminal cleaved, C-terminal cleaved and N -plus C-terminal-cleaved (double-cleaved)) were retrovirally expressed in CD4+ cells and the ability of the transduced cells to suppress Teff cell proliferation was measured.

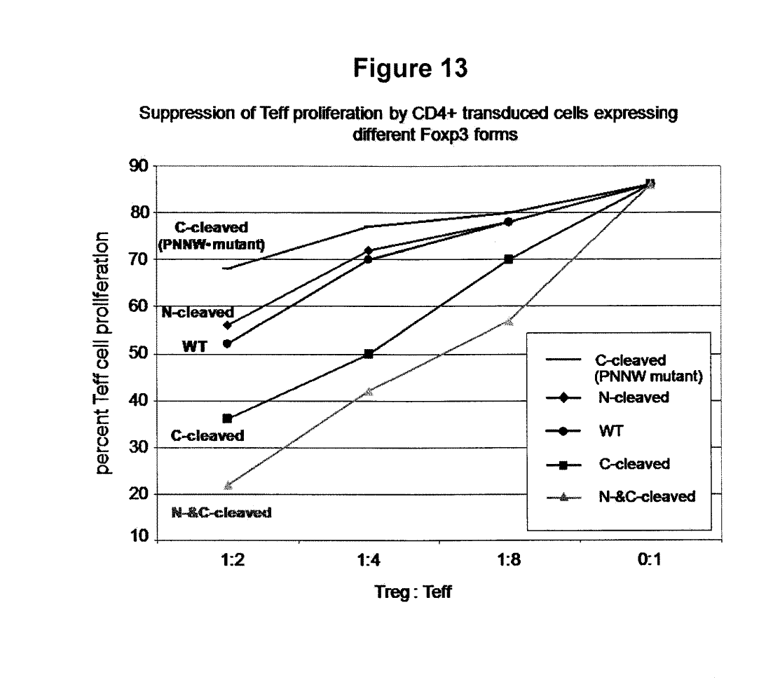

As shown in FIG. 13, CD4+ cells expressing C-cleaved or double-cleaved Foxp3 suppressed Teff cell proliferation stronger than both WTFoxp3 and N-terminal-cleaved Foxp3. The loss in the suppression ability of cells expressing the C-cleaved mutant in which RKKR (SEQ ID NO: 179) was replaced with the unrelated amino acids PNNW (SEQ ID NO: 184) highlights the importance of the 4 basic amino acids RKKR (SEQ ID NO: 179) in the function of Foxp3. By analogy to other FoxP subfamily members, KR of RKKR (SEQ ID NO: 179) represents DNA contact points. Interestingly, despite the absence of these two DNA contact points, the loss of function in the C-cleaved PNNW (SEQ ID NO: 184) mutant (417-aa) does not appear to be due to a general failure to bind to DNA but rather due to an inability of the terminal amino acids in making proper contacts, with DNA or other proteins. C-cleaved PNNW (SEQ ID NO: 184) mutant was found in the chromatin fraction at a similar level to WTFoxp3 when retrovirally expressed in CD4+ cells. While WT-Foxp3 was used in these experiments, WT-Foxp3 is not present in the cells as a homogenous population, but rather as a mixture of cleaved (N- and C-cleaved) and uncleaved forms. Hence, the functionality of the different forms of Foxp3 is best demonstrated using engineered mutants that mimic N-terminal or C-terminal cleaved Foxp3s. Partial loss of suppressive activity in N-cleaved Foxp3 indicates the N-terminal end may have role in the C-terminal recognition and cleavage by the PC(s).

### Example VII

### Contrasting Effects of Foxp3 Mutants on the Development of Experimental IBD

To test the effects of the different Foxp3 mutants in vivo, RAG−/− (C57BL/6) mice were injected with 1×10⁵ transduced Thy1.1+CD4+ cells expressing either WT-Foxp3 or a mutant Foxp3 (RKKR• (SEQ ID NO: 179), QNKR• (SEQ ID NO: 180), QNKR-- (SEQ ID NO: 180)) or empty vector (control), plus 1×10⁶ CD4+CD25− Teff cells. Additional mice received CD4+CD25− Teff cells alone. The weights of the animals in each group were monitored weekly for 45 days.

Mice that received cells expressing empty vector (MINR1) or no Treg progressively lost weight and succumbed to disease, whereas mice receiving cells expressing WT-Foxp3 showed minor weight loss but survived (p<0.01 vs. MINR1 or no Treg groups) (FIG. 14A). In contrast to use of WT-Foxp3, mice receiving short-Foxp3 (RKKR•) (SEQ ID NO: 179) continued to gain weight (p<0.05 vs. WT-Foxp3) (FIG. 14A). Histologic analysis of duodenal sections showed mild cell mononuclear cell infiltration and edema within villi of mice receiving WT-Foxp3, whereas mice receiving short-Foxp3 (RKKR•) (SEQ ID NO: 179) had essentially normal histology (FIG. 14B). Both groups showed considerable numbers of Foxp3+ mononuclear cells by immunoperoxidase staining, suggesting differences in weight loss were not due to difference in cell recruitment to inflamed gut tissues (FIG. 14B).

Use of differing QNKR (SEQ ID NO: 181) mutants also had contrasting effects on weight loss in this model (FIG. 14C). Mice receiving the short Foxp3 mutant (QNKR•) (SEQ ID NO: 181) showed negligible weight loss, whereas those receiving cells expressing the long Foxp3 mutant (QNKR--) (SEQ ID NO: 181) did significantly worse (p<0.05). Histology showed minor mononuclear cell infiltration and edema in mice receiving the short Foxp3 mutant, whereas the long Foxp3 mutant led to marked mononuclear cell infiltration of villous processes (FIG. 14D). Again, both groups showed accumulation of Foxp3+ mononuclear cells, suggesting differences in weight loss were not due to difference in cell recruitment (FIG. 14D).

The study was terminated at day 45 and splenocytes from each group were analyzed for Thy1.1 and Foxp3 expression. Flow cytometry showed most splenic Thy1.1+ cells expressed Foxp3, consistent with survival of transferred Foxp3+ cells throughout the study. When the number of spleen cells from each group was quantitated, (RKKR•) (SEQ ID NO: 179) group yielded the least number of total T cells (p<0.05), consistent with the absence of inflammatory disease in this group (FIG. 14E). Overall, these data demonstrate short-Foxp3 is the active form of Foxp3 and that strategies yielding higher levels of active Foxp3 will be of therapeutic importance.

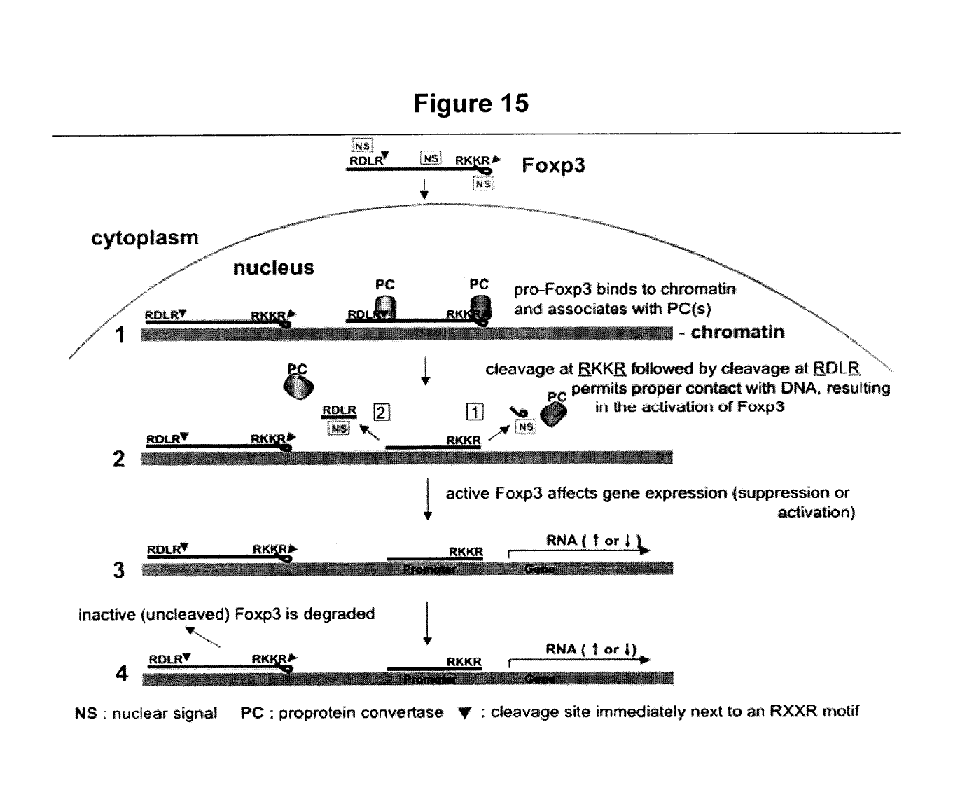

The results obtained have lead to the construction of a proposed mechanism of biochemical activation of Foxp3 (FIG. 15). The model of Foxp3 activation involves several structural changes in Foxp3 to achieve functionality (FIG. 15). The main steps towards Foxp3 function include (1) nuclear transport; (2) association with chromatin; (3) cleavage by the PC(s) to remove the N-terminal and C-terminal ends through proteolytic cleavage at the RXXR (SEQ ID NO: 180) sites. In addition, there is currently evidence (4) for the specific degradation of unprocessed chromatin-bound Foxp3. FIG. 15 is a schematic diagram showing the current state of our knowledge on the mechanism of Foxp3 activation (note, for simplicity, neither homo- or heterodimer formation of Foxp3 nor its association with histone acetyltransferases or histone deacetylases are indicated). It is possible that C-terminal processing allows certain structural changes in Foxp3 leading to more efficient cleavage of the N-terminal end, which is supported by the more efficient cleavage of C-cleaved Foxp3 (RKKR·) (SEQ ID NO: 179).

### REFERENCES

1. Allan, S. E., L. Passerini, R. Bacchetta, N. Crellin, M. Dai, P. C. Orban, S. F. Ziegler, M. G. Roncarolo, and M. K. Levings. 2005. The role of 2 FOXP3 isoforms in the generation of human CD4+ Tregs. J Clin Invest 115:3276-3284.
2. Takahashi, S., K. Kasai, K. Hatsuzawa, N. Kitamura, Y. Misumi, Y. Ikehara, K. Murakami, and K. Nakayama. 1993. A mutation of Furin causes the lack of precursor-processing activity in human colon carcinoma LoVo cells. Biochem Biophys Res Commun 195:1019-1026.
3. Smith, E. L., H. M. Finney, A. M. Nesbitt, F. Ramsdell, and M. K. Robinson. 2006. Splice variants of human FOXP3 are functional inhibitors of human CD4+ T-cell activation. Immunology 119:203-211.
4. Cokol, M., R. Nair, and B. Rost. 2000. Finding nuclear localization signals. EMBO Rep 1:411-415.
5. Powell, B. R., N. R. Buist, and P. T. Stenzel. 1982. An X-linked syndrome of diarrhea, polyendocrinopathy, and fatal infection in infancy. J Pediatr 100:731-737.
6. Gambineri, E., T. R. Torgerson, and H. D. Ochs. 2003. Immune dysregulation, polyendocrinopathy, enteropathy, and X-linked inheritance (IPEX), a syndrome of systemic autoimmunity caused by mutations of FOXP3, a critical regulator of T-cell homeostasis. Curr Opin Rheumatol 15:430-435.
7. Bennett, C. L., J. Christie, F. Ramsdell, M. E. Brunkow, P. J. Ferguson, L. Whitesell, T. E. Kelly, F. T. Saulsbury, P. F. Chance, and H. D. Ochs. 2001. The immune dysregulation, polyendocrinopathy, enteropathy, X-linked syndrome (IPEX) is caused by mutations of FOXP3. Nat Genet 27:20-21.
8. Wildin, R. S., S. Smyk-Pearson, and A. H. Filipovich. 2002. Clinical and molecular features of the immunodysregulation, polyendocrinopathy, enteropathy, X linked (IPEX) syndrome. J Med Genet 39:537-545.
9. Brunkow, M. E., E. W. Jeffery, K. A. Hjerrild, B. Paeper, L. B. Clark, S. A. Yasayko, J. E. Wilkinson, D. Galas, S. F. Ziegler, and F. Ramsdell. 2001. Disruption of a new forkhead/winged-helix protein, scurfin, results in the fatal lymphoproliferative disorder of the scurfy mouse. Nat Genet 27:68-73.
10. Clark, L. B., M. W. Appleby, M. E. Brunkow, J. E. Wilkinson, S. F. Ziegler, and F. Ramsdell. 1999. Cellular and molecular characterization of the scurfy mouse mutant. J Immunol 162:2546-2554.
11. Blair, P. J., S. J. Bultman, J. C. Haas, B. T. Rouse, J. E. Wilkinson, and V. L. Godfrey. 1994. CD4+CD8− T cells are the effector cells in disease pathogenesis in the scurfy (sf) mouse. J Immunol 153:3764-3774.
12. Wildin, R. S., F. Ramsdell, J. Peake, F. Faravelli, J. L. Casanova, N. Buist, E. Levy-Lahad, M. Mazzella, O. Goulet, L. Perroni, F. D. Bricarelli, G. Byrne, M. McEuen, S. Proll, M. Appleby, and M. E. Brunkow. 2001. X-linked neonatal diabetes mellitus, enteropathy and endocrinopathy syndrome is the human equivalent of mouse scurfy. Nat Genet 27:18-20.
13. Tang, Q., K. J. Henriksen, M. Bi, E. B. Finger, G. Szot, J. Ye, E. L. Masteller, H. McDevitt, M. Bonyhadi, and J. A. Bluestone. 2004. In vitro-expanded antigen-specific regulatory T cells suppress autoimmune diabetes. J Exp Med 199:1455-1465.
14. Jaeckel, E., H. von Boehmer, and M. P. Manns. 2005. Antigen-specific FoxP3-transduced T-cells can control established type 1 diabetes. Diabetes 54:306-310.
15. Ochs, H. D., S. F. Ziegler, and T. R. Torgerson. 2005. FOXP3 acts as a rheostat of the immune response. Immunol Rev 203:156-164.
16. Bennett, C. L., M. E. Brunkow, F. Ramsdell, K. C. O'Briant, Q. Zhu, R. L. Fuleihan, A. O. Shigeoka, H. D. Ochs, and P. F. Chance. 2001. A rare polyadenylation signal mutation of the FOXP3 gene (AAUAAA→AAUGAA) leads to the IPEX syndrome. Immunogenetics 53:435-439.
17. Kobayashi, I., R. Shiari, M. Yamada, N. Kawamura, M. Okano, A. Yara, A. Iguchi, N. Ishikawa, T. Ariga, Y. Sakiyama, H. D. Ochs, and K. Kobayashi. 2001. Novel mutations of FOXP3 in two Japanese patients with immune dysregulation, polyendocrinopathy, enteropathy, X linked syndrome (IPEX). J Med Genet 38:874-876.
18. Hori, S., T. Nomura, and S. Sakaguchi. 2003. Control of regulatory T cell development by the transcription factor Foxp3. Science 299:1057-1061.
19. Walker, M. R., B. D. Carson, G. T. Nepom, S. F. Ziegler, and J. H. Buckner. 2005. De novo generation of antigen-specific CD4+CD25+ regulatory T cells from human CD4+CD25− cells. Proc Natl Acad Sci USA 102:4103-4108.
20. Manavalan, J. S., S. Kim-Schulze, L. Scotto, A. J. Naiyer, G. Vlad, P. C. Colombo, C. Marboe, D. Mancini, R. Cortesini, and N. Suciu-Foca. 2004. Alloantigen specific CD8+CD28− FOXP3+ T suppressor cells induce ILT3+ ILT4+ tolerogenic endothelial cells, inhibiting alloreactivity. Int Immunol 16:1055-1068.
21. Fontenot, J. D., M. A. Gavin, and A. Y. Rudensky. 2003. Foxp3 programs the development and function of CD4+CD25+ regulatory T cells. Nat Immunol 4:330-336.
22. Coffer, P. J., and B. M. Burgering. 2004. Forkhead-box transcription factors and their role in the immune system. Nat Rev Immunol 4:889-899.
23. Carson, B. D., J. E. Lopes, D. M. Soper, and S. F. Ziegler. 2006. Insights into transcriptional regulation by FOXP3. Front Biosci 11:1607-1619.
24. Chamberlain, N. L., E. D. Driver, and R. L. Miesfeld. 1994. The length and location of CAG trinucleotide repeats in the androgen receptor N-terminal domain affect transactivation function. Nucleic Acids Res 22:3181-3186.
25. Stroud, J. C., Y. Wu, D. L. Bates, A. Han, K. Nowick, S. Paabo, H. Tong, and L. Chen. 2006. Structure of the forkhead domain of FOXP2 bound to DNA. Structure 14:159-166.
26. Li, S., J. Weidenfeld, and E. E. Morrisey. 2004. Transcriptional and DNA binding activity of the Foxp1/2/4 family is modulated by heterotypic and homotypic protein interactions. Mol Cell Biol 24:809-822.
27. McCarty, A. S., G. Kleiger, D. Eisenberg, and S. T. Smale. 2003. Selective dimerization of a C2H2 zinc finger subfamily. Mol Cell 11:459-470.
28. Khattri, R., T. Cox, S. A. Yasayko, and F. Ramsdell. 2003. An essential role for Scurfin in CD4+CD25+ T regulatory cells. Nat Immunol 4:337-342.
29. Wu, Y., M. Borde, V. Heissmeyer, M. Feuerer, A. D. Lapan, J. C. Stroud, D. L. Bates, L. Guo, A. Han, S. F. Ziegler, D. Mathis, C. Benoist, L. Chen, and A. Rao. 2006. FOXP3 controls regulatory T cell function through cooperation with NFAT. Cell 126:375-387.
30. Chen, C., E. A. Rowell, R. M. Thomas, W. W. Hancock, and A. D. Wells. 2006. Transcriptional regulation by Foxp3 is associated with direct promoter occupancy and modulation of histone acetylation. J Biol Chem. 281: 36828-36834.
31. Bettelli, E., M. Dastrange, and M. Oukka. 2005. Foxp3 interacts with nuclear factor of activated T cells and NF-kappa B to repress cytokine gene expression and effector functions of T helper cells. Proc Natl Acad Sci USA 102:5138-5143.
32. Morgan, B., L. Sun, N. Avitahl, K. Andrikopoulos, T. Ikeda, E. Gonzales, P. Wu, S. Neben, and K. Georgopoulos. 1997. Aiolos, a lymphoid restricted transcription factor that interacts with Ikaros to regulate lymphocyte differentiation. Embo J 16:2004-2013.
33. Molloy, S. S., P. A. Bresnahan, S. H. Leppla, K. R. Klimpel, and G. Thomas. 1992. Human Furin is a calcium-dependent serine endoprotease that recognizes the sequence Arg-X-X-Arg and efficiently cleaves anthrax toxin protective antigen. J Biol Chem 267:16396-16402.
34. Walker, J. A., S. S. Molloy, G. Thomas, T. Sakaguchi, T. Yoshida, T. M. Chambers, and Y. Kawaoka. 1994. Sequence specificity of furin, a proprotein-processing endoprotease, for the hemagglutinin of a virulent avian influenza virus. J Virol 68:1213-1218.
35. Krysan, D. J., N. C. Rockwell, and R. S. Fuller. 1999. Quantitative characterization of furin specificity. Energetics of substrate discrimination using an internally consistent set of hexapeptidyl methylcoumarinamides. J Biol Chem 274:23229-23234.
36. Jackson, R. S., J. W. Creemers, S. Ohagi, M. L. Raffin-Sanson, L. Sanders, C. T. Montague, J. C. Hutton, and S. O'Rahilly. 1997. Obesity and impaired prohormone processing associated with mutations in the human prohormone convertase 1 gene. Nat Genet 16:303-306.
37. Roebroek, A. J., L. Umans, I. G. Pauli, E. J. Robertson, F. van Leuven, W. J. Van de Ven, and D. B. Constam. 1998. Failure of ventral closure and axial rotation in embryos lacking the proprotein convertase Furin. Development 125:4863-4876.
38. Constam, D. B., and E. J. Robertson. 2000. SPC4/PACE4 regulates a TGFbeta signaling network during axis formation. Genes Dev 14:1146-1155.
39. Furuta, M., H. Yano, A. Zhou, Y. Rouille, J. J. Hoist, R. Carroll, M. Ravazzola, L. Orci, H. Furuta, and D. F. Steiner. 1997. Defective prohormone processing and altered pancreatic islet morphology in mice lacking active SPC2. Proc Natl Acad Sci USA 94:6646-6651.
40. Mbikay, M., H. Tadros, N. Ishida, C. P. Lerner, E. De Lamirande, A. Chen, M. El-Alfy, Y. Clermont, N. G. Seidah, M. Chretien, C. Gagnon, and E. M. Simpson. 1997. Impaired fertility in mice deficient for the testicular germ-cell protease PC4. Proc Natl Acad Sci USA 94:6842-6846.
41. Smeekens, S. P., A. S. Avruch, J. LaMendola, S. J. Chan, and D. F. Steiner. 1991. Identification of a cDNA encoding a second putative prohormone convertase related to PC2 in AtT20 cells and islets of Langerhans. Proc Natl Acad Sci USA 88:340-344.
42. Seidah, N. G., M. Marcinkiewicz, S. Benjannet, L. Gaspar, G. Beaubien, M. G. Mattei, C. Lazure, M. Mbikay, and M. Chretien. 1991. Cloning and primary sequence of a mouse candidate prohormone convertase PC1 homologous to PC2, Furin, and Kex2: distinct chromosomal localization and messenger RNA distribution in brain and pituitary compared to PC2. Mol Endocrinol 5:111-122.
43. Steiner, D. F., S. P. Smeekens, S. Ohagi, and S. J. Chan. 1992. The new enzymology of precursor processing endoproteases. J Biol Chem 267:23435-23438.
44. Zhou, Y., and I. Lindberg. 1993. Purification and characterization of the prohormone convertase PC1(PC3). J Biol Chem 268:5615-5623.
45. Munzer, J. S., A. Basak, M. Zhong, A. Mamarbachi, J. Hamelin, D. Savaria, C. Lazure, G. N. Hendy, S. Benjannet, M. Chretien, and N. G. Seidah. 1997. In vitro characterization of the novel proprotein convertase PC7. J Biol Chem 272:19672-19681.
46. Ozkaynak, E., P. N. Schnegelsberg, D. F. Jin, G. M. Clifford, F. D. Warren, E. A. Drier, and H. Oppermann. 1992. Osteogenic protein-2. A new member of the transforming growth factor-beta superfamily expressed early in embryogenesis. J Biol Chem 267:25220-25227.
47. Cui, Y., F. Jean, G. Thomas, and J. L. Christian. 1998. BMP-4 is proteolytically activated by furin and/or PC6 during vertebrate embryonic development. Embo J 17:4735-4743.
48. Dubois, C. M., F. Blanchette, M. H. Laprise, R. Leduc, F. Grondin, and N. G. Seidah. 2001. Evidence that furin is an authentic transforming growth factor-beta1-converting enzyme. Am J Pathol 158:305-316.
49. Thomas, G. 2002. Furin at the cutting edge: from protein traffic to embryogenesis and disease. Nat Rev Mol Cell Biol 3:753-766.
50. Vidricaire, G., J. B. Denault, and R. Leduc. 1993. Characterization of a secreted form of human furin endoprotease. Biochem Biophys Res Commun 195:1011-1018.
51. Lee, R., P. Kermani, K. K. Teng, and B. L. Hempstead. 2001. Regulation of cell survival by secreted proneurotrophins. Science 294:1945-1948.
52. Khatib, A. M., G. Siegfried, A. Prat, J. Luis, M. Chretien, P. Metrakos, and N. G. Seidah. 2001. Inhibition of proprotein convertases is associated with loss of growth and tumorigenicity of HT-29 human colon carcinoma cells: importance of insulin-like growth factor-1 (IGF-1) receptor processing in IGF-1-mediated functions. J Biol Chem 276:30686-30693.
53. Schneider, P., S. L. Street, O. Gaide, S. Hertig, A. Tardivel, J. Tschopp, L. Runkel, K. Alevizopoulos, B. M. Ferguson, and J. Zonana. 2001. Mutations leading to X-linked hypohidrotic ectodermal dysplasia affect three major functional domains in the tumor necrosis factor family member ectodysplasin-A. J Biol Chem 276:18819-18827.
54. Hensbergen, P. J., D. Verzijl, C. I. Balog, R. Dijkman, R. C. van der Schors, E. M. van der Raaij-Helmer, M. J. van der Plas, R. Leurs, A. M. Deelder, M. J. Smit, and C. P. Tensen. 2004. Furin is a chemokine-modifying enzyme: in vitro and in vivo processing of CXCL10 generates a C-terminally truncated chemokine retaining full activity. J Biol Chem 279:13402-13411.
55. Pesu, M., L. Muul, Y. Kanno, and J. J. O'Shea. 2006. Proprotein convertase furin is preferentially expressed in T helper 1 cells and regulates interferon gamma. Blood 108:983-985.
56. St Germain, C., G. Croissandeau, J. Mayne, J. M. Baltz, M. Chretien, and M. Mbikay. 2005. Expression and transient nuclear translocation of proprotein convertase 1 (PC1) during mouse preimplantation embryonic development. Mol Reprod Dev 72:483-493.
57. Kemper, B. 2004. Structural basis for the role in protein folding of conserved proline-rich regions in cytochromes P450. Toxicol Appl Pharmacol 199:305-315.
58. Nilsson, I., and G. von Heijne. 1998. Breaking the camel's back: proline-induced turns in a model transmembrane helix. J Mol Biol 284:1185-1189.
59. Duckert, P., S. Brunak, and N. Blom. 2004. Prediction of proprotein convertase cleavage sites. Protein Eng Des Sel 17:107-112.
60. Zhu, X., N. S. Lamango, and I. Lindberg. 1996. Involvement of a polyproline helix-like structure in the interaction of 7B2 with prohormone convertase 2. J Biol Chem 271:23582-23587.
61. Lange, A., R. E. Mills, C. J. Lange, M. Stewart, S. E. Devine, and A. H. Corbett. 2006. Classical nuclear localization signals: Definition, function, and interaction with importin alpha. J Biol Chem.
62. Takahashi, S., T. Nakagawa, K. Kasai, T. Banno, S. J. Duguay, W. J. Van de Ven, K. Murakami, and K. Nakayama. 1995. A second mutant allele of furin in the processing-incompetent cell line, LoVo. Evidence for involvement of the homo B domain in autocatalytic activation. J Biol Chem 270:26565-26569.
63. Lopes, J. E., T. R. Torgerson, L. A. Schubert, S. D. Anover, E. L. Ocheltree, H. D. Ochs, and S. F. Ziegler. 2006. Analysis of FOXP3 reveals multiple domains required for its function as a transcriptional repressor. J Immunol 177:3133-3142.
64. Kong, M., E. A. Barnes, V. Ollendorff, and D. J. Donoghue. 2000. Cyclin F regulates the nuclear localization of cyclin B1 through a cyclin-cyclin interaction. Embo J 19:1378-1388.
65. Turpin, P., R. T. Hay, and C. Dargemont. 1999. Characterization of IkappaBalpha nuclear import pathway. J Biol Chem 274:6804-6812.
66. Elliott, G., and P. O'Hare. 2000. Cytoplasm-to-nucleus translocation of a herpesvirus tegument protein during cell division. J Virol 74:2131-2141.
67. Lee, I., L. Wang, A. D. Wells, M. E. Dorf, E. Ozkaynak, and W. W. Hancock. 2005. Recruitment of Foxp3+ T regulatory cells mediating allograft tolerance depends on the CCR4 chemokine receptor. J Exp Med 201:1037-1044.
68. Hancock, W. W., M. H. Sayegh, X. G. Zheng, R. Peach, P. S. Linsley, and L. A. Turka. 1996. Costimulatory function and expression of CD40 ligand, CD80, and CD86 in vascularized murine cardiac allograft rejection. Proc Natl Acad Sci USA 93:13967-13972.
69. Pear, W. S., G. P. Nolan, M. L. Scott, and D. Baltimore. 1993. Production of high-titer helper-free retroviruses by transient transfection. Proc Natl Acad Sci USA 90:8392-8396.
70. Thomas, R. M, Gao, L., and Wells, A. D. 2005. Signals from CD28 induce stable epigenetic modification of the IL-2 promoter. J Immunol 174:4639-4646.
71. Hancock, W. W., Sayegh, M. H., Zheng, X. G., Peach, R., Linsley, P. S., and Turka, L. A. 1996. Costimulatory function and expression of CD40 ligand, CD80, and CD86 in vascularized murine cardiac allograft rejection. Proc. Natl Acad Sci USA 93: 13967-13972.
72. Zheng, S. G., Meng, L., Wang, J., H., Watanabe, M., Barr, M. L., Cramer, D. V., Gray, J. D., Horwitz, D. A. 2006. Transfer of regulatory T cells generated ex vivo modifies graft rejection through induction of tolerogenic CD4+CD25+ cells in the recipient. Int Immunol 18:279-289.
73. Tao, R., E. de Zoeten, E. Ozkaynak, C. Chen, L. Wang, P. M. Porrett, B. Li, L. A. Turka, E. N. Olson, M. I. Greene, A. D. Wells, and W. W. Hancock. 2007. Deacetylase inhibition promotes the generation and function of regulatory T cells. Nat. Med. [Epub ahead of print] PMID: 17922010
74. von der Decken, A., and G. M. Andersson. 1977. Template properties of rat liver chromatin after restricted protein supply; transcription by homologous ribonucleic acid polymerase I and II. J Nutr. 107:949-958.
75. Mbikay, M., N. G. Seidah, and M. Chretien. 2001. Neuroendocrine secretory protein 7B2: structure, expression and functions. Biochem J357:329-342.
76. Burton, K. 1956. A study of the conditions and mechanism of the diphenylamine reaction for the colorimetric estimation of deoxyribonucleic acid. Biochem J 62:315-323. 22.
77. T. Curiel et al., Nature Medicine (2004) 10(9): 942-949.
78. D. Wolf et al., Clinical Cancer Res. (2005) 11:8326-8331.
79. A. El Andaloussi et al., J. Neurooncology (2007) 83:145-152.
80. A. El Andaloussi et al., J. Neurosurgery (2006) 105:430-437.
81. R. P. Peterson et al., Cancer (2006) 107:2866-2872.
82. S. Hinz et al., Cancer Res (2007) 67(1):8344-8350.
83. Nishiya N et al., Molecular Immunology (2004) 41:855-861.
84. Schreiber, E. et al., Nucleic Acids Res. (1989) 17:6419.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. A method for identifying agents which affect the formation of cleaved Foxp3 comprising:
a) administering said agent to a cell expressing Foxp3 and enzymes responsible for cleavage thereof,
b) determining levels of Foxp3 cleavage product, if any, relative to an untreated cell, and
c) identifying those agents which modulate the formation of said Foxp3 cleavage product.

2. The method of claim 1, wherein said Foxp3 cleavage product is selected from the group consisting of N-terminal cleaved Foxp3, C-terminal cleaved Foxp3, and double-cleaved short Foxp3.

3. The method of claim 1, wherein said agent alters the expression level or function of a proprotein convertase enzyme, thereby modulating Foxp3 activity or Treg function.

4. The method of claim 1, wherein said agent alters the cellular localization of a proprotein convertase which cleaves Foxp3.

5. The method of claim 1, wherein said agent modulates formation of a peptide selected from the group consisting of SEQ ID NO: 8, SEQ ID NO: 11, SEQ ID NO: 15, SEQ ID NO: 20 and SEQ ID NO: 24.

6-26. (canceled)

27. An siRNA composition comprising at least one nucleotide sequence selected from the group consisting of SEQ ID: 25-SEQ ID NO: 174 in a pharmaceutically acceptable carrier for delivery to a patient with cancer.

28. A kit for practicing the method of claim 1, comprising at least one antibody for detecting Foxpeptide or the N-terminal peptide of Foxp3, wherein said antibody is specific for a sequence selected from the group consisting of SEQ ID NO: 8, SEQ ID NO: 11, SEQ ID NO: 15, SEQ ID NO: 20, and SEQ ID NO: 24, said kit optionally containing fragments of Foxp3 for use as positive controls.

29. An isolated nucleic acid encoding a peptide selected from the group consisting of SEQ ID NO: 8, SEQ ID NO: 11, SEQ ID NO: 15, SEQ ID NO: 20, and SEQ ID NO: 24, or a nucleic acid complementary thereto.

30. A peptide encoded by the nucleic acid of claim 29.

31. A method for the treatment of an autoimmune disease in a patient in need thereof comprising increasing Treg activity via introduction of an effective amount of at least one nucleic acid of claim 30, such that Treg activity is increased.

32. The method of claim 31, wherein said nucleic acid is delivered to said cell in a vector selected from the group consisting of adenoviral vectors, plasmids, adeno-associated viral vectors, retroviral vectors, hybrid adeno-associated virus vectors, lentivirus vectors, herpes simplex virus vectors, and vaccine vectors, or in an antibody studded liposome, wherein said antibody has immunospecificity for a target cell.

33. A method of treating autoimmunity in a patient in need thereof comprising administering an effective amount of a at least one peptide of claim 30, said peptides optionally being contained in a liposome.

34. The method of claim 33, wherein said agent is administered ex vivo to isolated cells for a time sufficient to stimulate Treg production, after which said cells are reinfused into a patient.

35. A method for assessing regulatory T cell activation in a test subject, comprising:
a) providing a biological sample obtained from said test subject, said sample comprising Foxp3 cleavage products as claimed in claim 2;
b) contacting said sample with an agent having affinity for said cleavage product,
c) comparing the amount of said Foxp3 cleavage product from said test subject with levels of Foxp3 cleavage products obtained from a normal subject, wherein an alteration of in the amount of Foxp3 cleavage product in the sample, relative to the normal subject is indicative of altered regulatory T cell activation.

36. The method of claim 35, wherein said Foxp3 cleavage product level is reduced indicating said patient has autoimmune disease or is undergoing transplant rejection.

37. The method of claim 35, wherein the method is repeated several times over a course of treatment, said patient is a transplant patient and Foxp3 cleavage product levels are utilized to determine effective levels of immunosuppressive agents for alleviation of autoimmune symptoms.

38. The method as claimed in claim 37, wherein said patient is being weaned off of immunosuppressive therapy.

39. The method of claim 35, wherein said Foxp3 cleavage product level is elevated indicating said patient has cancer.

40. The method of claim 35 further comprising assessing inflammatory cytokine levels in the sample.

41. The method of claim 35, further comprising identifying T cell specific markers present on T cells in said sample, said markers being selected from the group consisting of GITR, CTLA-4, and CD25.

42. The method of claim 39, wherein the ratio of said SEQ ID NO: 8 or SEQ ID NO: 20 relative to SEQ ID NO: 2 is determined.

43. The method of claim 35, wherein the expression ratio of SEQ ID NO: 15 to SEQ ID NO: 2 is determined.


SEQUENCE LISTING (193 sequences)

SEQ ID NO: 1 - protein, 429 aa, Artificial Sequence
  Synthetic Sequence
MPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 2 - protein, 431 aa, Artificial Sequence
  Synthetic Sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 3 - DNA, 1290 bp, Artificial Sequence
  Synthetic Sequence
atgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgcccttga

SEQ ID NO: 4 - DNA, 1296 bp, Artificial Sequence
  Synthetic Sequence
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 5 - DNA, 36 bp, Artificial Sequence
  Synthetic Sequence
agccaacgccccaacaagtgctccaatccctgccct

SEQ ID NO: 6 - protein, 12 aa, Artificial Sequence
  Synthetic Sequence
SQRPNKCSNPCP

SEQ ID NO: 7 - DNA, 42 bp, Artificial Sequence
  Synthetic Sequence
agccagaggcccagcaggtgttccaaccctacacctggcccc

SEQ ID NO: 8 - protein, 14 aa, Artificial Sequence
  Synthetic Sequence
SQRPSRCSNPTPGP

SEQ ID NO: 9 - protein, 417 aa, Artificial Sequence
  Synthetic Sequence
MPNPRPAKPMAPSLALGPSPGVLPSWKTAPKGSELLGTRGSGGPFQGRDLRSGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKR

SEQ ID NO: 10 - DNA, 1254 bp, Artificial Sequence
  Synthetic Sequence
atgcccaaccctaggccagccaagcctatggctccttccttggcccttggcccatccccaggagtcttgccaagctggaagactgcacccaagggctcagaacttctagggaccaggggctctgggggacccttccaaggtcgggacctgcgaagtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggtga

SEQ ID NO: 11 - protein, 417 aa, Artificial Sequence
  Synthetic Sequence
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKR

SEQ ID NO: 12 - DNA, 1254 bp, Artificial Sequence
  Synthetic Sequence
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggtga

SEQ ID NO: 13 - protein, 366 aa, Artificial Sequence
  Synthetic Sequence
SGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKR

SEQ ID NO: 14 - DNA, 1101 bp, Artificial Sequence
  Synthetic Sequence
agtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggtga

SEQ ID NO: 15 - protein, 366 aa, Artificial Sequence
  Synthetic Sequence
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKR

SEQ ID NO: 16 - DNA, 1101 bp, Artificial Sequence
  Synthetic Sequence
ggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggtga

SEQ ID NO: 17 - DNA, 153 bp, Artificial Sequence
  Synthetic Sequence
atgcccaaccctaggccagccaagcctatggctccttccttggcccttggcccatccccaggagtcttgccaagctggaagactgcacccaagggctcagaacttctagggaccaggggctctgggggacccttccaaggtcgggacctgcga

SEQ ID NO: 18 - protein, 51 aa, Artificial Sequence
  Synthetic Sequence
MPNPRPAKPMAPSLALGPSPGVLPSWKTAPKGSELLGTRGSGGPFQGRDLR

SEQ ID NO: 19 - DNA, 153 bp, Artificial Sequence
  Synthetic Sequence
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcga

SEQ ID NO: 20 - protein, 51 aa, Artificial Sequence
  Synthetic Sequence
MPNPRPGKPSAPSLALGPSPGASPSWRAAPKASDLLGARGPGGTFQGRDLR

SEQ ID NO: 21 - DNA, 1137 bp, Artificial Sequence
  Synthetic Sequence
agtggggcccacacctcttcttccttgaaccccctgccaccatcccagctgcagctgcctacagtgcccctagtcatggtggcaccgtctggggcccgactaggtccctcaccccacctacaggcccttctccaggacagaccacacttcatgcatcagctctccactgtggatgcccatgcccagacccctgtgctccaagtgcgtccactggacaacccagccatgatcagcctcccaccaccttctgctgccactggggtcttctccctcaaggcccggcctggcctgccacctgggatcaatgtggccagtctggaatgggtgtccagggagccagctctactctgcaccttcccacgctcgggtacacccaggaaagacagcaaccttttggctgcaccccaaggatcctacccactgctggcaaatggagtctgcaagtggcctggttgtgagaaggtcttcgaggagccagaagagtttctcaagcactgccaagcagatcatctcctggatgagaaaggcaaggcccagtgcctcctccagagagaagtggtgcagtctctggagcagcagctggagctggaaaaggagaagctgggagctatgcaggcccacctggctgggaagatggcgctggccaaggctccatctgtggcctcaatggacaagagctcttgctgcatcgtagccaccagtactcagggcagtgtgctcccggcctggtctgctcctcgggaggctccagacggcggcctgtttgcagtgcggaggcacctctggggaagccatggcaatagttccttcccagagttcttccacaacatggactacttcaagtaccacaatatgcgaccccctttcacctatgccacccttatccgatgggccatcctggaagccccggagaggcagaggacactcaatgaaatctaccattggtttactcgcatgttcgcctacttcagaaaccaccccgccacctggaagaatgccatccgccacaacctgagcctgcacaagtgctttgtgcgagtggagagcgagaagggagcagtgtggaccgtagatgaatttgagtttcgcaagaagaggagccaacgccccaacaagtgctccaatccctgcccttga

SEQ ID NO: 22 - protein, 378 aa, Artificial Sequence
  Synthetic Sequence
SGAHTSSSLNPLPPSQLQLPTVPLVMVAPSGARLGPSPHLQALLQDRPHFMHQLSTVDAHAQTPVLQVRPLDNPAMISLPPPSAATGVFSLKARPGLPPGINVASLEWVSREPALLCTFPRSGTPRKDSNLLAAPQGSYPLLANGVCKWPGCEKVFEEPEEFLKHCQADHLLDEKGKAQCLLQREVVQSLEQQLELEKEKLGAMQAHLAGKMALAKAPSVASMDKSSCCIVATSTQGSVLPAWSAPREAPDGGLFAVRRHLWGSHGNSSFPEFFHNMDYFKYHNMRPPFTYATLIRWAILEAPERQRTLNEIYHWFTRMFAYFRNHPATWKNAIRHNLSLHKCFVRVESEKGAVWTVDEFEFRKKRSQRPNKCSNPCP

SEQ ID NO: 23 - DNA, 1143 bp, Artificial Sequence
  Synthetic Sequence
ggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 24 - protein, 380 aa, Artificial Sequence
  Synthetic Sequence
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 25 - DNA, 21 bp, Artificial Sequence
  Synthetic Sequence
cctgaaagctaatggagaatt

SEQ ID NO: 26 - DNA, 21 bp, Artificial Sequence
  Synthetic Sequence
caataaccctggatggaaatt

SEQ ID NO: 27 - DNA, 21 bp, Artificial Sequence
  Synthetic Sequence
ccctaatgatgatgggaaatt

SEQ ID NO: 28 - DNA, 21 bp, Artificial Sequence
  Synthetic Sequence
ggagagaaccctataggtatt

SEQ ID NO: 29 - DNA, 21 bp, Artificial Sequence
  Synthetic Sequence
gctgaaagagaacgggatatt

SEQ ID NO: 30 - DNA, 21 bp, Artificial Sequence
  Synthetic Sequence
tgatgatgatcgtgtgatatt

SEQ ID NO: 31 - DNA, 21 bp, Artificial Sequence
  Synthetic Sequence
acttggactttgagaattatt

SEQ ID NO: 32 - DNA, 21 bp, Artificial Sequence
  Synthetic Sequence
agaaagagtgtgttgtaaatt

SEQ ID NO: 33 - DNA, 21 bp, Artificial Sequence
  Synthetic Sequence
agaaagaagtaaacgttcatt

SEQ ID NO: 34 - DNA, 21 bp, Artificial Sequence
  Synthetic Sequence
aagtaaacgttcagctctatt

SEQ ID NO: 35 - DNA, 21 bp, Artificial Sequence
  Synthetic Sequence
ccacaaacgagaacaaacatt

SEQ ID NO: 36 - DNA, 21 bp, Artificial Sequence
  Synthetic Sequence
gaccagatgtgcaggagaatt

SEQ ID NO: 37 - DNA, 21 bp, Artificial Sequence
  Synthetic Sequence
ccaaagctctggtggattttt

SEQ ID NO: 38 - DNA, 21 bp, Artificial Sequence
  Synthetic Sequence
caagagaaccctaaggagatt

SEQ ID NO: 39 - DNA, 21 bp, Artificial Sequence
  Synthetic Sequence
gaacagtgcaaaagcgaaatt

SEQ ID NO: 40 - DNA, 21 bp, Artificial Sequence
  Synthetic Sequence
caatggtacttgcaagatatt

SEQ ID NO: 41 - DNA, 21 bp, Artificial Sequence
  Synthetic Sequence
cagatgtgcaggagaaatttt

SEQ ID NO: 42 - DNA, 21 bp, Artificial Sequence
  Synthetic Sequence
ccagaaggcttttgaatattt

SEQ ID NO: 43 - DNA, 21 bp, Artificial Sequence
  Synthetic Sequence
gtggatatttacagtgcaatt

SEQ ID NO: 44 - DNA, 21 bp, Artificial Sequence
  Synthetic Sequence
gaatagtcgatttggattttt

SEQ ID NO: 45 - DNA, 21 bp, Artificial Sequence
  Synthetic Sequence
ggagcgtgcctgagaagaatt

SEQ ID NO: 46 - DNA, 21 bp, Artificial Sequence
  Synthetic Sequence
gaagaaagagtgtgttgtatt

SEQ ID NO: 47 - DNA, 21 bp, Artificial Sequence
  Synthetic Sequence
gtacttggactttgagaattt

SEQ ID NO: 48 - DNA, 21 bp, Artificial Sequence
  Synthetic Sequence
aagagaaccctaaggagaatt

SEQ ID NO: 49 - DNA, 21 bp, Artificial Sequence
  Synthetic Sequence
ggagcaggcttgatggtgatt

SEQ ID NO: 50 - DNA, 21 bp, Artificial Sequence
  Synthetic Sequence
cctaatggctttaagaacttt

SEQ ID NO: 51 - DNA, 21 bp, Artificial Sequence
  Synthetic Sequence
gaacaaaccttcccagctttt

SEQ ID NO: 52 - DNA, 21 bp, Artificial Sequence
  Synthetic Sequence
tgtgatacctgtttggcaatt

SEQ ID NO: 53 - DNA, 21 bp, Artificial Sequence
  Synthetic Sequence
gcaagcaaataatcacaaatt

SEQ ID NO: 54 - DNA, 21 bp, Artificial Sequence
  Synthetic Sequence
gagcaggcttgatggtgaatt

SEQ ID NO: 55 - DNA, 21 bp, Artificial Sequence
  Synthetic Sequence
gggctgaacaacagtatgatt

SEQ ID NO: 56 - DNA, 21 bp, Artificial Sequence
  Synthetic Sequence
gggcaaaggagttgttatctt

SEQ ID NO: 57 - DNA, 21 bp, Artificial Sequence
  Synthetic Sequence
gaagaggggtggagaagattt

SEQ ID NO: 58 - DNA, 21 bp, Artificial Sequence
  Synthetic Sequence
cagaaggtctcgaaggagttt

SEQ ID NO: 59 - DNA, 21 bp, Artificial Sequence
  Synthetic Sequence
gctgaacaacagtatgaaatt

SEQ ID NO: 60 - DNA, 21 bp, Artificial Sequence
  Synthetic Sequence
ggaatcacacggacatttatt

SEQ ID NO: 61 - DNA, 21 bp, Artificial Sequence
  Synthetic Sequence
ggttggagttgcatacaattt

SEQ ID NO: 62 - DNA, 21 bp, Artificial Sequence
  Synthetic Sequence
gggagataattgtgactgttt

SEQ ID NO: 63 - DNA, 21 bp, Artificial Sequence
  Synthetic Sequence
ggagaagttatcattgaaatt

SEQ ID NO: 64 - DNA, 21 bp, Artificial Sequence
  Synthetic Sequence
cattgaaattccaacaagatt

SEQ ID NO: 65 - DNA, 21 bp, Artificial Sequence
  Synthetic Sequence
gcaaaagcgaaaaggcaattt

SEQ ID NO: 66 - DNA, 21 bp, Artificial Sequence
  Synthetic Sequence
tgatatgggctgaacaacatt

SEQ ID NO: 67 - DNA, 21 bp, Artificial Sequence
  Synthetic Sequence
ctggacacgtggatatttatt

SEQ ID NO: 68 - DNA, 21 bp, Artificial Sequence
  Synthetic Sequence
gcgctgacctgcacaatgatt

SEQ ID NO: 69 - DNA, 21 bp, Artificial Sequence
  Synthetic Sequence
gaaagctaatggagaagtttt

SEQ ID NO: 70 - DNA, 21 bp, Artificial Sequence
  Synthetic Sequence
tctaagggactcagcactatt

SEQ ID NO: 71 - DNA, 21 bp, Artificial Sequence
  Synthetic Sequence
cactaaatctcttcaatgatt

SEQ ID NO: 72 - DNA, 21 bp, Artificial Sequence
  Synthetic Sequence
cacaatgactgcacggagatt

SEQ ID NO: 73 - DNA, 21 bp, Artificial Sequence
  Synthetic Sequence
tgtacaatttgaagcaacatt

SEQ ID NO: 74 - DNA, 21 bp, Artificial Sequence
  Synthetic Sequence
ccacacaagagaaccctaatt

SEQ ID NO: 75 - DNA, 21 bp, Artificial Sequence
  Synthetic Sequence
agacaaaggctgttagagatt

SEQ ID NO: 76 - DNA, 21 bp, Artificial Sequence
  Synthetic Sequence
cagcaaggatccagacgaatt

SEQ ID NO: 77 - DNA, 21 bp, Artificial Sequence
  Synthetic Sequence
ggaggaagggacagagctatt

SEQ ID NO: 78 - DNA, 21 bp, Artificial Sequence
  Synthetic Sequence
ccataggagctgtggatgatt

SEQ ID NO: 79 - DNA, 21 bp, Artificial Sequence
  Synthetic Sequence
tggaaagcctggaaggtgatt

SEQ ID NO: 80 - DNA, 21 bp, Artificial Sequence
  Synthetic Sequence
actacatgctggaagtatatt

SEQ ID NO: 81 - DNA, 21 bp, Artificial Sequence
  Synthetic Sequence
ggacagagctagaatcagttt

SEQ ID NO: 82 - DNA, 21 bp, Artificial Sequence
  Synthetic Sequence
agcaatggcacctgaataatt

SEQ ID NO: 83 - DNA, 21 bp, Artificial Sequence
  Synthetic Sequence
cggtggtggtagtggatgatt

SEQ ID NO: 84 - DNA, 21 bp, Artificial Sequence
  Synthetic Sequence
cagacgaagtggaaacagatt

SEQ ID NO: 85 - DNA, 21 bp, Artificial Sequence
  Synthetic Sequence
cagcaatggcacctgaatatt

SEQ ID NO: 86 - DNA, 21 bp, Artificial Sequence
  Synthetic Sequence
gaaaatacctgcacgatgatt

SEQ ID NO: 87 - DNA, 21 bp, Artificial Sequence
  Synthetic Sequence
gcagtagacatcagggacatt

SEQ ID NO: 88 - DNA, 21 bp, Artificial Sequence
  Synthetic Sequence
caatcaagtttgtaggagttt

SEQ ID NO: 89 - DNA, 21 bp, Artificial Sequence
  Synthetic Sequence
ccggaaagccaaggaggaatt

SEQ ID NO: 90 - DNA, 21 bp, Artificial Sequence
  Synthetic Sequence
gctatgacctcaactctaatt

SEQ ID NO: 91 - DNA, 21 bp, Artificial Sequence
  Synthetic Sequence
gagagtgccatgagtggaatt

SEQ ID NO: 92 - DNA, 21 bp, Artificial Sequence
  Synthetic Sequence
gaagtatatttgagccagatt

SEQ ID NO: 93 - DNA, 21 bp, Artificial Sequence
  Synthetic Sequence
gcatggaggcagtggcgtttt

SEQ ID NO: 94 - DNA, 21 bp, Artificial Sequence
  Synthetic Sequence
aggcagtggcgttcaacaatt

SEQ ID NO: 95 - DNA, 21 bp, Artificial Sequence
  Synthetic Sequence
aggaagggacagagctagatt

SEQ ID NO: 96 - DNA, 21 bp, Artificial Sequence
  Synthetic Sequence
gaccagatgacgatgggaatt

SEQ ID NO: 97 - DNA, 21 bp, Artificial Sequence
  Synthetic Sequence
gtgcagagtgggtcaccaatt

SEQ ID NO: 98 - DNA, 21 bp, Artificial Sequence
  Synthetic Sequence
ggacattgcacccaactattt

SEQ ID NO: 99 - DNA, 21 bp, Artificial Sequence
  Synthetic Sequence
cggatgtggagaatggcaatt

SEQ ID NO: 100 - DNA, 21 bp, Artificial Sequence
  Synthetic Sequence
gcgatgtgcaggagagatctt

SEQ ID NO: 101 - DNA, 21 bp, Artificial Sequence
  Synthetic Sequence
gggtgacggtggtggtagttt

SEQ ID NO: 102 - DNA, 21 bp, Artificial Sequence
  Synthetic Sequence
tggatgacggagtggaacatt

SEQ ID NO: 103 - DNA, 21 bp, Artificial Sequence
  Synthetic Sequence
catcggagccggaaagccatt

SEQ ID NO: 104 - DNA, 21 bp, Artificial Sequence
  Synthetic Sequence
gcaaggatccagacgaagttt

SEQ ID NO: 105 - DNA, 21 bp, Artificial Sequence
  Synthetic Sequence
agtggatgacggagtggaatt

SEQ ID NO: 106 - DNA, 21 bp, Artificial Sequence
  Synthetic Sequence
caggagagatcgcggctgttt

SEQ ID NO: 107 - DNA, 21 bp, Artificial Sequence
  Synthetic Sequence
ctacgtcagtcccgtgttatt

SEQ ID NO: 108 - DNA, 21 bp, Artificial Sequence
  Synthetic Sequence
ccctggaggtcctgtggaatt

SEQ ID NO: 109 - DNA, 21 bp, Artificial Sequence
  Synthetic Sequence
ggatggacctggagatgtctt

SEQ ID NO: 110 - DNA, 21 bp, Artificial Sequence
  Synthetic Sequence
gaatgtggcttccaatcaatt

SEQ ID NO: 111 - DNA, 21 bp, Artificial Sequence
  Synthetic Sequence
acgggaaggaggagcagattt

SEQ ID NO: 112 - DNA, 21 bp, Artificial Sequence
  Synthetic Sequence
cagacagcatggaggcagttt

SEQ ID NO: 113 - DNA, 21 bp, Artificial Sequence
  Synthetic Sequence
acgtcagtcccgtgttaaatt

SEQ ID NO: 114 - DNA, 21 bp, Artificial Sequence
  Synthetic Sequence
gacatcagggacagacaaatt

SEQ ID NO: 115 - DNA, 21 bp, Artificial Sequence
  Synthetic Sequence
ctacatgctggaagtatattt

SEQ ID NO: 116 - DNA, 21 bp, Artificial Sequence
  Synthetic Sequence
agccaaggaggaagggacatt

SEQ ID NO: 117 - DNA, 21 bp, Artificial Sequence
  Synthetic Sequence
ccaaggaggaagggacagatt

SEQ ID NO: 118 - DNA, 21 bp, Artificial Sequence
  Synthetic Sequence
agggacagagctagaatcatt

SEQ ID NO: 119 - DNA, 21 bp, Artificial Sequence
  Synthetic Sequence
acgcaatgtgactgggcgatt

SEQ ID NO: 120 - DNA, 21 bp, Artificial Sequence
  Synthetic Sequence
gcaacggaggccaacacaatt

SEQ ID NO: 121 - DNA, 21 bp, Artificial Sequence
  Synthetic Sequence
caccataggagctgtggattt

SEQ ID NO: 122 - DNA, 21 bp, Artificial Sequence
  Synthetic Sequence
gctgaagaccctggagcattt

SEQ ID NO: 123 - DNA, 21 bp, Artificial Sequence
  Synthetic Sequence
acaaaggctgttagagagttt

SEQ ID NO: 124 - DNA, 21 bp, Artificial Sequence
  Synthetic Sequence
tatatttgagccagaggaatt

SEQ ID NO: 125 - DNA, 21 bp, Artificial Sequence
  Synthetic Sequence
gcccaaagacatcgggaaatt

SEQ ID NO: 126 - DNA, 21 bp, Artificial Sequence
  Synthetic Sequence
tggaacagcaggtggcaaatt

SEQ ID NO: 127 - DNA, 21 bp, Artificial Sequence
  Synthetic Sequence
ggactaaacgggacgtgtatt

SEQ ID NO: 128 - DNA, 21 bp, Artificial Sequence
  Synthetic Sequence
cggcagaagtgcacggagttt

SEQ ID NO: 129 - DNA, 21 bp, Artificial Sequence
  Synthetic Sequence
acacacagatgaatgacaatt

SEQ ID NO: 130 - DNA, 21 bp, Artificial Sequence
  Synthetic Sequence
gcactatagcaccgagaattt

SEQ ID NO: 131 - DNA, 21 bp, Artificial Sequence
  Synthetic Sequence
agaatgacgtggagaccattt

SEQ ID NO: 132 - DNA, 21 bp, Artificial Sequence
  Synthetic Sequence
cgagtgggtcctagagatttt

SEQ ID NO: 133 - DNA, 21 bp, Artificial Sequence
  Synthetic Sequence
tggcaaagcgacggactaatt

SEQ ID NO: 134 - DNA, 21 bp, Artificial Sequence
  Synthetic Sequence
gcagatgggtttaatgacttt

SEQ ID NO: 135 - DNA, 21 bp, Artificial Sequence
  Synthetic Sequence
gctcagggccagaaggtcttt

SEQ ID NO: 136 - DNA, 21 bp, Artificial Sequence
  Synthetic Sequence
gtggcaaagcgacggactatt

SEQ ID NO: 137 - DNA, 21 bp, Artificial Sequence
  Synthetic Sequence
caacggtgtctgtggtgtatt

SEQ ID NO: 138 - DNA, 21 bp, Artificial Sequence
  Synthetic Sequence
gaacatgacagctgcaacttt

SEQ ID NO: 139 - DNA, 21 bp, Artificial Sequence
  Synthetic Sequence
ccagcgaagccaacaactatt

SEQ ID NO: 140 - DNA, 21 bp, Artificial Sequence
  Synthetic Sequence
actatagcaccgagaatgatt

SEQ ID NO: 141 - DNA, 21 bp, Artificial Sequence
  Synthetic Sequence
ggtacacacagatgaatgatt

SEQ ID NO: 142 - DNA, 21 bp, Artificial Sequence
  Synthetic Sequence
gcaaccagaatgagaagcatt

SEQ ID NO: 143 - DNA, 21 bp, Artificial Sequence
  Synthetic Sequence
cggaagtgcatcatcgacatt

SEQ ID NO: 144 - DNA, 21 bp, Artificial Sequence
  Synthetic Sequence
gcgagtgggtcctagagattt

SEQ ID NO: 145 - DNA, 21 bp, Artificial Sequence
  Synthetic Sequence
ccaacagtgtggcacggaatt

SEQ ID NO: 146 - DNA, 21 bp, Artificial Sequence
  Synthetic Sequence
ggacttggcaggcaattattt

SEQ ID NO: 147 - DNA, 21 bp, Artificial Sequence
  Synthetic Sequence
cagcagtggcaaccagaattt

SEQ ID NO: 148 - DNA, 21 bp, Artificial Sequence
  Synthetic Sequence
aatgagaagcagatcgtgatt

SEQ ID NO: 149 - DNA, 21 bp, Artificial Sequence
  Synthetic Sequence
ggaagtgcatcatcgacattt

SEQ ID NO: 150 - DNA, 21 bp, Artificial Sequence
  Synthetic Sequence
cagccaggccacatgactatt

SEQ ID NO: 151 - DNA, 21 bp, Artificial Sequence
  Synthetic Sequence
ccgcagatgggtttaatgatt

SEQ ID NO: 152 - DNA, 21 bp, Artificial Sequence
  Synthetic Sequence
acacgtgggctgtgcgcattt

SEQ ID NO: 153 - DNA, 21 bp, Artificial Sequence
  Synthetic Sequence
gagaagaaccacccggacttt

SEQ ID NO: 154 - DNA, 21 bp, Artificial Sequence
  Synthetic Sequence
agaagaaccacccggactttt

SEQ ID NO: 155 - DNA, 21 bp, Artificial Sequence
  Synthetic Sequence
gctgcaactgcgacggctatt

SEQ ID NO: 156 - DNA, 21 bp, Artificial Sequence
  Synthetic Sequence
acgactgacttgcggcagatt

SEQ ID NO: 157 - DNA, 21 bp, Artificial Sequence
  Synthetic Sequence
ccacactggccacgacctatt

SEQ ID NO: 158 - DNA, 21 bp, Artificial Sequence
  Synthetic Sequence
gtacagacctcgaagccagtt

SEQ ID NO: 159 - DNA, 21 bp, Artificial Sequence
  Synthetic Sequence
cagcgaagccaacaactattt

SEQ ID NO: 160 - DNA, 21 bp, Artificial Sequence
  Synthetic Sequence
ccttggaccctgtggagcatt

SEQ ID NO: 161 - DNA, 21 bp, Artificial Sequence
  Synthetic Sequence
ctacacagggcacggcatttt

SEQ ID NO: 162 - DNA, 21 bp, Artificial Sequence
  Synthetic Sequence
cggacttggcaggcaattatt

SEQ ID NO: 163 - DNA, 21 bp, Artificial Sequence
  Synthetic Sequence
cgactgacttgcggcagaatt

SEQ ID NO: 164 - DNA, 21 bp, Artificial Sequence
  Synthetic Sequence
tcaccgagcccaaagacattt

SEQ ID NO: 165 - DNA, 21 bp, Artificial Sequence
  Synthetic Sequence
aagtacagtggctggaacatt

SEQ ID NO: 166 - DNA, 21 bp, Artificial Sequence
  Synthetic Sequence
ccaaccacatccacatctatt

SEQ ID NO: 167 - DNA, 21 bp, Artificial Sequence
  Synthetic Sequence
gcagtggcaaccagaatgatt

SEQ ID NO: 168 - DNA, 21 bp, Artificial Sequence
  Synthetic Sequence
gtggcaaccagaatgagaatt

SEQ ID NO: 169 - DNA, 21 bp, Artificial Sequence
  Synthetic Sequence
ccctggaggccaataagaatt

SEQ ID NO: 170 - DNA, 21 bp, Artificial Sequence
  Synthetic Sequence
tcatatggctacgggcttttt

SEQ ID NO: 171 - DNA, 21 bp, Artificial Sequence
  Synthetic Sequence
cggctcaccctgtcctatatt

SEQ ID NO: 172 - DNA, 21 bp, Artificial Sequence
  Synthetic Sequence
agtgggtcctagagattgatt

SEQ ID NO: 173 - DNA, 21 bp, Artificial Sequence
  Synthetic Sequence
actatgggacgctgaccaatt

SEQ ID NO: 174 - DNA, 21 bp, Artificial Sequence
  Synthetic Sequence
gcaccgagaatgacgtggatt

SEQ ID NO: 175 - DNA, 21 bp, Artificial Sequence
  Synthetic Sequence
gaacccaatgcccaaccctag

SEQ ID NO: 176 - DNA, 22 bp, Artificial Sequence
  Synthetic Sequence
ttcttggttttgaggtcaaggg

SEQ ID NO: 177 - DNA, 37 bp, Artificial Sequence
  Synthetic Sequence
gtgacccgaattcatgcccaaccctaggccagccaag

SEQ ID NO: 178 - DNA, 39 bp, Artificial Sequence
  Synthetic Sequence
gaggttggaattctcacctcttcttgcaaactcaaattc

SEQ ID NO: 179 - protein, 4 aa, Artificial Sequence
  Synthetic Sequence
RKKR

SEQ ID NO: 180 - protein, 4 aa, Artificial Sequence
  Synthetic Sequence
RXXR

SEQ ID NO: 181 - protein, 4 aa, Artificial Sequence
  Synthetic Sequence
QNKR

SEQ ID NO: 182 - protein, 4 aa, Artificial Sequence
  Synthetic Sequence
QNKS

SEQ ID NO: 183 - protein, 4 aa, Artificial Sequence
  Synthetic Sequence
RDLR

SEQ ID NO: 184 - protein, 4 aa, Artificial Sequence
  Synthetic Sequence
PNNW

SEQ ID NO: 185 - protein, 6 aa, Artificial Sequence
  Synthetic Sequence
LLRVKR

SEQ ID NO: 186 - protein, 11 aa, Artificial Sequence
  Synthetic Sequence
QRPNKCSNPCP

SEQ ID NO: 187 - protein, 4 aa, Artificial Sequence
  Synthetic Sequence
RXKR

SEQ ID NO: 188 - protein, 5 aa, Artificial Sequence
  Synthetic Sequence
RKKRS

SEQ ID NO: 189 - protein, 4 aa, Artificial Sequence
  Synthetic Sequence
QKRR

SEQ ID NO: 190 - protein, 5 aa, Artificial Sequence
  Synthetic Sequence
QKRRP

SEQ ID NO: 191 - protein, 5 aa, Artificial Sequence
  Synthetic Sequence
RDLRS

SEQ ID NO: 192 - protein, 4 aa, Artificial Sequence
  Synthetic Sequence
HDLH

SEQ ID NO: 193 - protein, 5 aa, Artificial Sequence
  Synthetic Sequence
HDLHS